SEQUENCE LISTING (181 sequences)

SEQ ID NO: 1 - protein, 17 aa, Artificial Sequence
  cell penetrating peptide
RXRXRXRXRXRXRXRXX

SEQ ID NO: 2 - protein, 22 aa, Artificial Sequence
  cell penetrating peptide
RRRRRRRQLKIWFQNRRMKWKK

SEQ ID NO: 3 - DNA, 21 bp, Artificial Sequence
  antisense oligo
cagcagcagagtcttcatcat

SEQ ID NO: 4 - DNA, 32 bp, Artificial Sequence
  oligonucleotide
gcaaacctgcatgactcacgcccaaagaatgc

SEQ ID NO: 5 - DNA, 22 bp, Artificial Sequence
  oligonucleotide
tgcatgactcacgcccaaagaa

SEQ ID NO: 6 - DNA, 22 bp, Artificial Sequence
  oligonucleotide
gcaaacctgcatgactcacgcc

SEQ ID NO: 7 - DNA, 35 bp, Artificial Sequence
  oligonucleotide
ccctgattggacatttcctggagctcattgtctga

SEQ ID NO: 8 - DNA, 22 bp, Artificial Sequence
  oligonucleotide
ggacatttcctggagctcattg

SEQ ID NO: 9 - DNA, 22 bp, Artificial Sequence
  oligonucleotide
ccctgattggacatttcctgga

SEQ ID NO: 10 - DNA, 40 bp, Artificial Sequence
  oligonucleotide
ctcagagggccatcatggtctcattgcacactcctgggag

SEQ ID NO: 11 - DNA, 22 bp, Artificial Sequence
  oligonucleotide
gggccatcatggtctcattgca

SEQ ID NO: 12 - DNA, 19 bp, Artificial Sequence
  oligonucleotide
cagagggccatcatggtct

SEQ ID NO: 13 - DNA, 183 bp, Artificial Sequence
  oligonucleotide
ccggtcacggaccctgtgcccatgctgctgctcgtcacggaccctgtgcccatgctgctgctcctggcgacgccgcgttgtgggcactgggaggcgccgtatttatagcgctccgttcgcgcacacacctttggggctggctgcaaacctgcatgactcacgcccaaagaatgccgcggaaag

SEQ ID NO: 14 - DNA, 20 bp, Artificial Sequence
  oligonucleotide
gcccaaagaatgccgcggaa

SEQ ID NO: 15 - DNA, 19 bp, Artificial Sequence
  oligonucleotide
gggctggctgcaaacctgc

SEQ ID NO: 16 - DNA, 20 bp, Artificial Sequence
  oligonucleotide
gctccgttcgcgcacacacc

SEQ ID NO: 17 - DNA, 20 bp, Artificial Sequence
  oligonucleotide
ccgcgttgtgggcactggga

SEQ ID NO: 18 - DNA, 20 bp, Artificial Sequence
  oligonucleotide
tgctgctcctggcgacgccg

SEQ ID NO: 19 - DNA, 20 bp, Artificial Sequence
  oligonucleotide
ccctgtgcccatgctgctgc

SEQ ID NO: 20 - DNA, 20 bp, Artificial Sequence
  oligonucleotide
cggtcacggaccctgtgccc

SEQ ID NO: 21 - DNA, 32 bp, Artificial Sequence
  oligonucleotide
cagcagagtcttcatcatgcctccaattcttg

SEQ ID NO: 22 - DNA, 22 bp, Artificial Sequence
  oligonucleotide
ttcatcatgcctccaattctgg

SEQ ID NO: 23 - DNA, 22 bp, Artificial Sequence
  oligonucleotide
cagcagcagagtcttcatcatg

SEQ ID NO: 24 - DNA, 269 bp, Artificial Sequence
  oligonucleotide
cttgactttcaagacgcggaggcaacagcagccagcccttcacaccgaatccatctgcatcctagtgggagacttgggccgggctgcctgtgcattcaggggtggaagtagtggaagccaggaaggacaattccttcggagagtagagagggttcgcagtggcccgaggagcaggcttcccagagaaagtccctttggaaggatgagtcctcttgctctgccaggccctccacactgcccatcccctctaagccgacacgaggctgccc

SEQ ID NO: 25 - DNA, 20 bp, Artificial Sequence
  oligonucleotide
tgcccatcccctctaagccg

SEQ ID NO: 26 - DNA, 20 bp, Artificial Sequence
  oligonucleotide
tgccaggccctccacactgc

SEQ ID NO: 27 - DNA, 20 bp, Artificial Sequence
  oligonucleotide
cttgctctgccaggccctcc

SEQ ID NO: 28 - DNA, 20 bp, Artificial Sequence
  oligonucleotide
cgcagtggcccgaggagcag

SEQ ID NO: 29 - DNA, 20 bp, Artificial Sequence
  oligonucleotide
gccgggctgcctgtgcattc

SEQ ID NO: 30 - DNA, 20 bp, Artificial Sequence
  oligonucleotide
ggagacttgggccgggctgc

SEQ ID NO: 31 - DNA, 20 bp, Artificial Sequence
  oligonucleotide
gcagccagcccttcacaccg

SEQ ID NO: 32 - DNA, 20 bp, Artificial Sequence
  oligonucleotide
gacgcggaggcaacagcagc

SEQ ID NO: 33 - DNA, 51 bp, Artificial Sequence
  oligonucleotide
cagcaggcccatctggacactcagtagcacattaacaaggagtgcctctca

SEQ ID NO: 34 - DNA, 23 bp, Artificial Sequence
  oligonucleotide
cacattaacaaggagtgcctctc

SEQ ID NO: 35 - DNA, 23 bp, Artificial Sequence
  oligonucleotide
tctggacactcagtagcacatta

SEQ ID NO: 36 - DNA, 48 bp, Artificial Sequence
  oligonucleotide
gtctttgcacgcctccatttgccatcacagaaccaacaggacgatggg

SEQ ID NO: 37 - DNA, 22 bp, Artificial Sequence
  oligonucleotide
cgcctccatttgccatcacaga

SEQ ID NO: 38 - DNA, 20 bp, Artificial Sequence
  oligonucleotide
cacgcctccatttgccatca

SEQ ID NO: 39 - DNA, 140 bp, Artificial Sequence
  oligonucleotide
ccatttgccatcacagaaccaacaggacgatgggttccccttcctgaaatggttcacatccaaagcccgctcagcccagcaggcccatctggacactcagtagcacattaacaaggagtgcctctcatgagaagtaatca

SEQ ID NO: 40 - DNA, 20 bp, Artificial Sequence
  oligonucleotide
cagcaggcccatctggacac

SEQ ID NO: 41 - DNA, 20 bp, Artificial Sequence
  oligonucleotide
tccaaagcccgctcagccca

SEQ ID NO: 42 - DNA, 20 bp, Artificial Sequence
  oligonucleotide
ggttcacatccaaagcccgc

SEQ ID NO: 43 - DNA, 20 bp, Artificial Sequence
  oligonucleotide
cgatgggttccccttcctga

SEQ ID NO: 44 - DNA, 48 bp, Artificial Sequence
  oligonucleotide
gtacggagagaagggcatcaagctgcggacgatgcgggacttgggaaa

SEQ ID NO: 45 - DNA, 22 bp, Artificial Sequence
  oligonucleotide
tgcggacgatgcgggacttggg

SEQ ID NO: 46 - DNA, 22 bp, Artificial Sequence
  oligonucleotide
gggcatcaagctgcggacgatg

SEQ ID NO: 47 - DNA, 42 bp, Artificial Sequence
  oligonucleotide
tatgaattctgttggcgggtgctggaaggccgggctgtggaa

SEQ ID NO: 48 - DNA, 22 bp, Artificial Sequence
  oligonucleotide
tgctggaaggccgggctgtgga

SEQ ID NO: 49 - DNA, 23 bp, Artificial Sequence
  oligonucleotide
tctgttggcgggtgctggaaggc

SEQ ID NO: 50 - DNA, 42 bp, Artificial Sequence
  oligonucleotide
cttcgccttgcgtgaggtttgccagccgggacacccagttaa

SEQ ID NO: 51 - DNA, 22 bp, Artificial Sequence
  oligonucleotide
ggtttgccagccgggacaccca

SEQ ID NO: 52 - DNA, 22 bp, Artificial Sequence
  oligonucleotide
tgcgtgaggtttgccagccggg

SEQ ID NO: 53 - DNA, 23 bp, Artificial Sequence
  oligonucleotide
gggagaggctgggcggagttggg

SEQ ID NO: 54 - DNA, 23 bp, Artificial Sequence
  oligonucleotide
gggagaggctgggcggagttggg

SEQ ID NO: 55 - DNA, 33 bp, Artificial Sequence
  oligonucleotide
ggttcaggaactcctcaagctggcggccaacca

SEQ ID NO: 56 - DNA, 23 bp, Artificial Sequence
  oligonucleotide
ggaactcctcaagctggcggcca

SEQ ID NO: 57 - DNA, 38 bp, Artificial Sequence
  oligonucleotide
tctatgatgctggacgcgcggctgaagtggtcctgcat

SEQ ID NO: 58 - DNA, 22 bp, Artificial Sequence
  oligonucleotide
gctggacgcgcggctgaagtgg

SEQ ID NO: 59 - DNA, 23 bp, Artificial Sequence
  oligonucleotide
cgcgcggctgaagtggtcctgca

SEQ ID NO: 60 - DNA, 38 bp, Artificial Sequence
  oligonucleotide
acactggtccttcatccgcaggcagcccgtggagttgt

SEQ ID NO: 61 - DNA, 23 bp, Artificial Sequence
  oligonucleotide
ccgcaggcagcccgtggagttgt

SEQ ID NO: 62 - DNA, 22 bp, Artificial Sequence
  oligonucleotide
tccttcatccgcaggcagcccg

SEQ ID NO: 63 - DNA, 39 bp, Artificial Sequence
  oligonucleotide
ggattcgtcgagctcccgccgcagcttagcctgggaggg

SEQ ID NO: 64 - DNA, 22 bp, Artificial Sequence
  oligonucleotide
tcccgccgcagcttagcctggg

SEQ ID NO: 65 - DNA, 22 bp, Artificial Sequence
  oligonucleotide
ttcgtcgagctcccgccgcagc

SEQ ID NO: 66 - DNA, 31 bp, Artificial Sequence
  oligonucleotide
aagtgtgggaagccaccgtggtgacccgcag

SEQ ID NO: 67 - DNA, 22 bp, Artificial Sequence
  oligonucleotide
ggaagccaccgtggtgacccgc

SEQ ID NO: 68 - DNA, 40 bp, Artificial Sequence
  oligonucleotide
ctttagcagctcgttgtatttcctggtcaacctctcagcg

SEQ ID NO: 69 - DNA, 22 bp, Artificial Sequence
  oligonucleotide
ggtttgccagccgggacaccca

SEQ ID NO: 70 - DNA, 22 bp, Artificial Sequence
  oligonucleotide
tgcgtgaggtttgccagccggg

SEQ ID NO: 71 - DNA, 38 bp, Artificial Sequence
  oligonucleotide
tacttattttccatcccccctgcctgccccccatagca

SEQ ID NO: 72 - DNA, 25 bp, Artificial Sequence
  oligonucleotide
ttccatcccccctgcctgcccccca

SEQ ID NO: 73 - DNA, 39 bp, Artificial Sequence; identical to SEQ ID NO: 63
  oligonucleotide

SEQ ID NO: 74 - DNA, 22 bp, Artificial Sequence
  oligonucleotide
tcccgccgcagcttagcctggg

SEQ ID NO: 75 - DNA, 22 bp, Artificial Sequence
  oligonucleotide
ttcgtcgagctcccgccgcagc

SEQ ID NO: 76 - DNA, 47 bp, Artificial Sequence
  oligonucleotide
gctttgttcttgggctctggctccgggcggagtcgttcccaccaggc

SEQ ID NO: 77 - DNA, 22 bp, Artificial Sequence
  oligonucleotide
ggcggagtcgttcccaccaggc

SEQ ID NO: 78 - DNA, 22 bp, Artificial Sequence
  oligonucleotide
ttgggctctggctccgggcgga

SEQ ID NO: 79 - DNA, 23 bp, Artificial Sequence
  oligonucleotide
gggagaggctgggcggagttggg

SEQ ID NO: 80 - DNA, 23 bp, Artificial Sequence
  oligonucleotide
gggagaggctgggcggagttggg

SEQ ID NO: 81 - DNA, 27 bp, Artificial Sequence
  oligonucleotide
gggaatggatccgttgacctgcaggtc

SEQ ID NO: 82 - DNA, 22 bp, Artificial Sequence
  oligonucleotide
gggaatggatccgttgacctgc

SEQ ID NO: 83 - DNA, 22 bp, Artificial Sequence
  oligonucleotide
gggcaatgggtggacagggcgg

SEQ ID NO: 84 - DNA, 21 bp, Artificial Sequence
  oligonucleotide
gggcaatgggtggacagggcg

SEQ ID NO: 85 - DNA, 37 bp, Artificial Sequence
  oligonucleotide
agggagaatctcctgtcgcagccacccctccccctgg

SEQ ID NO: 86 - DNA, 22 bp, Artificial Sequence
  oligonucleotide
tcgcagccacccctccccctgg

SEQ ID NO: 87 - DNA, 32 bp, Artificial Sequence
  oligonucleotide
gctgccatcccctgcccgcccatccgtcctgg

SEQ ID NO: 88 - DNA, 22 bp, Artificial Sequence
  oligonucleotide
tcccctgcccgcccatccgtcc

SEQ ID NO: 89 - DNA, 42 bp, Artificial Sequence
  oligonucleotide
ctccggcacctcccctccctggcttcctgggctccgtcgaaa

SEQ ID NO: 90 - DNA, 22 bp, Artificial Sequence
  oligonucleotide
cctggcttcctgggctccgtcg

SEQ ID NO: 91 - DNA, 22 bp, Artificial Sequence
  oligonucleotide
tccggcacctcccctccctggc

SEQ ID NO: 92 - DNA, 37 bp, Artificial Sequence
  oligonucleotide
gttgcgtgaagctcgtgctcatcaggcggcggttgcg

SEQ ID NO: 93 - DNA, 22 bp, Artificial Sequence
  oligonucleotide
tgctcatcaggcggcggttgcg

SEQ ID NO: 94 - DNA, 35 bp, Artificial Sequence
  oligonucleotide
ggatgagagcccagccctcccagccccatgtccag

SEQ ID NO: 95 - DNA, 22 bp, Artificial Sequence
  oligonucleotide
gcccagccctcccagccccatg

SEQ ID NO: 96 - DNA, 23 bp, Artificial Sequence
  oligonucleotide
gccctccacactgcccatcccct

SEQ ID NO: 97 - DNA, 23 bp, Artificial Sequence
  oligonucleotide
gccctccacactgcccatcccct

SEQ ID NO: 98 - DNA, 38 bp, Artificial Sequence
  oligonucleotide
cagcctgctnngtcccgcccactccgccctgcagcagc

SEQ ID NO: 99 - DNA, 22 bp, Artificial Sequence
  oligonucleotide
ccgcccactccgccctgcagca

SEQ ID NO: 100 - DNA, 33 bp, Artificial Sequence
  oligonucleotide
ctgcccactctcccaggtcagcagcagccccac

SEQ ID NO: 101 - DNA, 22 bp, Artificial Sequence
  oligonucleotide
tcccaggtcagcagcagcccca

SEQ ID NO: 102 - DNA, 43 bp, Artificial Sequence
  oligonucleotide
gtttacgtaatgctccagccccccgccgtgcccacttcgcaga

SEQ ID NO: 103 - DNA, 23 bp, Artificial Sequence
  oligonucleotide
gccccccgccgtgcccacttcgc

SEQ ID NO: 104 - DNA, 22 bp, Artificial Sequence
  oligonucleotide
tgctccagccccccgccgtgcc

SEQ ID NO: 105 - DNA, 41 bp, Artificial Sequence
  oligonucleotide
cctccagtgggatggtcaaggcagggaggcggtagcggctc

SEQ ID NO: 106 - DNA, 22 bp, Artificial Sequence
  oligonucleotide
ggcagggaggcggtagcggctc

SEQ ID NO: 107 - DNA, 23 bp, Artificial Sequence
  oligonucleotide
gggatggtcaaggcagggaggcg

SEQ ID NO: 108 - DNA, 37 bp, Artificial Sequence
  oligonucleotide
ggcaccgcgcagtgaccccctccctcccctccagtgg

SEQ ID NO: 109 - DNA, 22 bp, Artificial Sequence
  oligonucleotide
ccccctccctcccctccagtgg

SEQ ID NO: 110 - DNA, 22 bp, Artificial Sequence
  oligonucleotide
ggcaccgcgcagtgaccccctc

SEQ ID NO: 111 - DNA, 30 bp, Artificial Sequence
  oligonucleotide
gcccctgccgccaccccacccagagcctgc

SEQ ID NO: 112 - DNA, 22 bp, Artificial Sequence
  oligonucleotide
tgccgccaccccacccagagcc

SEQ ID NO: 113 - DNA, 22 bp, Artificial Sequence
  oligonucleotide
gcccctgccgccaccccaccca

SEQ ID NO: 114 - DNA, 28 bp, Artificial Sequence
  oligonucleotide
aagcttcccctcattcgcccacctggaa

SEQ ID NO: 115 - DNA, 22 bp, Artificial Sequence
  oligonucleotide
tcccctcattcgcccacctgga

SEQ ID NO: 116 - DNA, 29 bp, Artificial Sequence
  oligonucleotide
aggagagaccctgaggtgcggcgctggcg

SEQ ID NO: 117 - DNA, 22 bp, Artificial Sequence
  oligonucleotide
accctgaggtgcggcgctggcg

SEQ ID NO: 118 - DNA, 28 bp, Artificial Sequence
  oligonucleotide
gcggttgcgcggcccctggctcagctgc

SEQ ID NO: 119 - DNA, 22 bp, Artificial Sequence
  oligonucleotide
ttgcgcggcccctggctcagct

SEQ ID NO: 120 - DNA, 47 bp, Artificial Sequence
  oligonucleotide
gaaacccctccccctgcactccacccccaccatgcccctcccaagaa

SEQ ID NO: 121 - DNA, 22 bp, Artificial Sequence
  oligonucleotide
ccacccccaccatgcccctccc

SEQ ID NO: 122 - DNA, 22 bp, Artificial Sequence
  oligonucleotide
ccccctgcactccacccccacc

SEQ ID NO: 123 - DNA, 26 bp, Artificial Sequence
  oligonucleotide
gtgtccccttttcacctggcggccaa

SEQ ID NO: 124 - DNA, 23 bp, Artificial Sequence
  oligonucleotide
tccccttttcacctggcggccaa

SEQ ID NO: 125 - DNA, 26 bp, Artificial Sequence
  oligonucleotide
ggaccagcccccagcaccccatctgt

SEQ ID NO: 126 - DNA, 22 bp, Artificial Sequence
  oligonucleotide
ggaccagcccccagcaccccat

SEQ ID NO: 127 - DNA, 33 bp, Artificial Sequence
  oligonucleotide
catcaagctgcggacgatgcgggacttgggaaa

SEQ ID NO: 128 - DNA, 22 bp, Artificial Sequence
  oligonucleotide
tgcggacgatgcgggacttggg

SEQ ID NO: 129 - DNA, 33 bp, Artificial Sequence
  oligonucleotide
tcgtatgaattctgttggcgggtgctggaaggc

SEQ ID NO: 130 - DNA, 22 bp, Artificial Sequence
  oligonucleotide
ctgttggcgggtgctggaaggc

SEQ ID NO: 131 - DNA, 25 bp, Artificial Sequence
  oligonucleotide
ccacggctcctggctcccaccccct

SEQ ID NO: 132 - DNA, 23 bp, Artificial Sequence
  oligonucleotide
cacggctcctggctcccaccccc

SEQ ID NO: 133 - DNA, 39 bp, Artificial Sequence
  oligonucleotide
gtctatgatgctggacgcgcggctgaagtggtcctgcat

SEQ ID NO: 134 - DNA, 22 bp, Artificial Sequence
  oligonucleotide
gctggacgcgcggctgaagtgg

SEQ ID NO: 135 - DNA, 48 bp, Artificial Sequence
  oligonucleotide
gacgctaggatctgactgcggctcctccatggcagtgacccggaaggc

SEQ ID NO: 136 - DNA, 22 bp, Artificial Sequence
  oligonucleotide
ccatggcagtgacccggaaggc

SEQ ID NO: 137 - DNA, 21 bp, Artificial Sequence
  oligonucleotide
gcggctcctccatggcagtga

SEQ ID NO: 138 - DNA, 22 bp, Artificial Sequence
  oligonucleotide
tctgactgcggctcctccatgg

SEQ ID NO: 139 - DNA, 41 bp, Artificial Sequence
  oligonucleotide
tcaatatcgtccggggacagcatcaaatcatccattgcttg

SEQ ID NO: 140 - DNA, 22 bp, Artificial Sequence
  oligonucleotide
gcatcaaatcatccattgcttg

SEQ ID NO: 141 - DNA, 22 bp, Artificial Sequence
  oligonucleotide
gggacagcatcaaatcatccat

SEQ ID NO: 142 - protein, 16 aa, Artificial Sequence
  Synthetic sequence
RQLKIWFQNRRMKWKK

SEQ ID NO: 143 - DNA, 20 bp, Artificial Sequence
  oligonucleotide
ccctgctccccccuggcucc

SEQ ID NO: 144 - DNA, 19 bp, Artificial Sequence
  oligonucleotide
ccctgctccccncuggcuc

SEQ ID NO: 145 - DNA, 19 bp, Artificial Sequence
  oligonucleotide
ccctgctncccncuggcuc

SEQ ID NO: 146 - DNA, 16 bp, Artificial Sequence
  oligonucleotide
gccctgctucccncug

SEQ ID NO: 147 - DNA, 16 bp, Artificial Sequence
  oligonucleotide
gccctgctucccccug

SEQ ID NO: 148 - DNA, 16 bp, Artificial Sequence
  oligonucleotide
gccctgctucccccug

SEQ ID NO: 149 - DNA, 20 bp, Artificial Sequence
  oligonucleotide
ccctgctccccccuggcucc

SEQ ID NO: 150 - DNA, 20 bp, Artificial Sequence
  oligonucleotide
ccctgctccccccuggcucc

SEQ ID NO: 151 - DNA, 20 bp, Artificial Sequence
  oligonucleotide
cccugcucccccctggctcc

SEQ ID NO: 152 - DNA, 20 bp, Artificial Sequence
  oligonucleotide
ccctgcuccccccuggctcc

SEQ ID NO: 153 - DNA, 20 bp, Artificial Sequence
  oligonucleotide
cccugctcccccctggcucc

SEQ ID NO: 154 - DNA, 20 bp, Artificial Sequence
  oligonucleotide
cccugctcccccctggcucc

SEQ ID NO: 155 - DNA, 20 bp, Artificial Sequence
  oligonucleotide
cccugctcccccctggcucc

SEQ ID NO: 156 - DNA, 20 bp, Artificial Sequence
  oligonucleotide
ccctgctcccccctggcucc

SEQ ID NO: 157 - DNA, 20 bp, Artificial Sequence
  oligonucleotide
ccctgctccccccuggcucc

SEQ ID NO: 158 - DNA, 20 bp, Artificial Sequence
  oligonucleotide
ccctgctccccccuggcucc

SEQ ID NO: 159 - DNA, 20 bp, Artificial Sequence
  oligonucleotide
ccctgcuccccccuggcucc

SEQ ID NO: 160 - DNA, 20 bp, Artificial Sequence
  oligonucleotide
cccugcuccccccuggcucc

SEQ ID NO: 161 - DNA, 19 bp, Artificial Sequence
  oligonucleotide
cccugcttcccnctggcuc

SEQ ID NO: 162 - DNA, 19 bp, Artificial Sequence
  oligonucleotide
cccugcttcccnctggcuc

SEQ ID NO: 163 - DNA, 19 bp, Artificial Sequence
  oligonucleotide
cccugcttcccnctggcuc

SEQ ID NO: 164 - DNA, 19 bp, Artificial Sequence
  oligonucleotide
ccctgcttcccnctggctc

SEQ ID NO: 165 - DNA, 19 bp, Artificial Sequence
  oligonucleotide
cccugcttcccnctggcuc

SEQ ID NO: 166 - DNA, 19 bp, Artificial Sequence
  oligonucleotide
cccugcttcccnctggcuc

SEQ ID NO: 167 - DNA, 19 bp, Artificial Sequence
  oligonucleotide
cccugcttcccnctggcuc

SEQ ID NO: 168 - DNA, 19 bp, Artificial Sequence
  oligonucleotide
ccctgcttcccnctggctc

SEQ ID NO: 169 - DNA, 19 bp, Artificial Sequence
  oligonucleotide
ccctgcttcccncuggcuc

SEQ ID NO: 170 - DNA, 19 bp, Artificial Sequence
  oligonucleotide
ccctgcttcccncuggcuc

SEQ ID NO: 171 - DNA, 19 bp, Artificial Sequence
  oligonucleotide
ccctgcttcccncuggcuc

SEQ ID NO: 172 - DNA, 19 bp, Artificial Sequence
  oligonucleotide
ccctgcttcccnctggctc

SEQ ID NO: 173 - DNA, 20 bp, Artificial Sequence
  oligonucleotide
cccugctcccccctggcucc

SEQ ID NO: 174 - DNA, 20 bp, Artificial Sequence
  oligonucleotide
cccugctcccccctggcucc

SEQ ID NO: 175 - DNA, 20 bp, Artificial Sequence
  oligonucleotide
ccctgctcccccctggctcc

SEQ ID NO: 176 - DNA, 20 bp, Artificial Sequence
  oligonucleotide
cccugcuccccccuggcucc

SEQ ID NO: 177 - DNA, 20 bp, Artificial Sequence
  oligonucleotide
cccugcuccccccuggcucc

SEQ ID NO: 178 - DNA, 20 bp, Artificial Sequence
  oligonucleotide
cccugcuccccccuggcucc

SEQ ID NO: 179 - DNA, 20 bp, Artificial Sequence
  oligonucleotide
cccugctccccccuggcucc

SEQ ID NO: 180 - DNA, 20 bp, Artificial Sequence
  oligonucleotide
cccugcuccccccuggcucc

SEQ ID NO: 181 - DNA, 20 bp, Artificial Sequence
  oligonucleotide
ccctgctcccccctggctcc

(19) United States
(12) Patent Application Publication
(10) Pub. No.: US 2015/0010502 A1
(43) Pub. Date: Jan. 8, 2015

(54) Compositions and Methods for Suppressing Gene Expression of p53 and Clusterin

(71) Applicant: Larry J. Smith, Omaha, NE (US)

(72) Inventor: Larry J. Smith, Omaha, NE (US)

(21) Appl. No.: 14/232,452

(22) PCT Filed: Jul. 12, 2012
(86) PCT No.: PCT/US12/46463

§ 371 Date: Sep. 8, 2014

Related U.S. Application Data
(60) Provisional application No. 61/507,356, filed on Jul. 13, 2011.

(51) Int. Cl. C12N 15/113; A61K 45/06; A61K 31/713

(52) U.S. Cl. CPC C12N 15/1135; A61K 31/713; A61K 45/06; C12N 2320/30; C12N 2310/14
USPC 424/85.4; 514/44 A; 424/85.1; 435/375

(57) ABSTRACT

Compositions and methods for inhibition of p53 and clusterin expression in target cells for the treatment of disease are disclosed.

This application claims priority to U.S. Provisional Application No. 61/507,356 filed Jul. 13, 2011, which is incorporated herein by reference as though set forth in full.

### FIELD OF THE INVENTION

The present invention relates to the fields of medicine and nucleic acid based therapies. More specifically, the invention provides compositions and methods for modulating p53 and clusterin expression in target cells using antisense oligonucleotides (oligos).

### BACKGROUND OF THE INVENTION

Several publications and patent documents are cited within this application in order to more fully describe the state of the art to which this invention pertains. The disclosure of each of these citations is incorporated by reference herein. p53 is one of the most studied proteins known to be involved in cellular programming. It is mutated in more than half of all cancers where it typically appears in elevated levels compared to wild type p53.

Prior to the late 1980's, p53 was considered to be an oncogene largely on the basis of its pattern of expression in cells and the fact that transfection of p53 genes into cells promoted their transformation to a quasi-malignant phenotype. It was then discovered that the first p53 genes employed in such transfection studies were mutated and that wild type p53 genes, in contrast, inhibited transformation. (Lane D P and Benchimol S (1990) Genes & Dev 4: 1-8; Levine A J (1997) Cell 88: 323-331).

This discovery launched a massive research effort to characterize the tumor suppressor function of wild type p53 that has been in progress for over 20 years. During most of this time the overwhelmingly dominant view of the scientific community has been that the wild type p53 in those cancers that express it is functionally inactive and that the mutant p53 found in the majority of all cancers is essentially an inactivated form of the wild type p53 tumor suppressor. Over the last few years, however, evidence has been building in the scientific literature that mutant p53 exhibits novel gain-of-function properties associated with promotion of cancer, for example, by promoting resistance to the induction of p53-independent programmed cell death. Further, there is a substantial and growing body of evidence that endogenous wild type p53 in cancers maintains certain functions that can support the cancer including the repair of DNA damage (Tovar, C. et al. (2006) Proc Natl Acad Sci (USA) 103: 1888-1898; Janicke, R. U., et al., (2008) Cell Death Differ 15: 959-976).

The normal function of p53 includes protecting the body from defective cells that have undergone DNA damage, proto-oncogene activation or have developed mutations that promote certain non-malignant diseases associated with translational abnormalities such as the ribosomopathies (Narla et al., (2010) Blood 115: 3196-3205). Such protection is achieved by the defective cell undergoing either p53-dependent cell death (any of these stimuli) or p53-dependent cell cycle arrest and repair (repair being limited to DNA damage). In general the higher the level of damage and/or p53-inducing mutations the higher the level of p53 induction and the greater the likelihood that the affected cells will undergo p53-dependent programmed cell death rather than cell cycle arrest with the possibility of repair. As a consequence, if a pre-malignant cell is to progress to a cancer, it must inhibit p53-dependent programmed cell death (Asker et al., (1999) Biochem Biophys Res Comm 265: 1-6; Igney et al., (2002) Nature Rev Cancer 2: 277-288; Schmitt et al., (2002) Cancer Cell 1: 289-298; Schmitt et al., (2003) Nature Rev Cancer 3: 286-295). Mechanisms employed by cancers to inhibit p53-dependent cell death include: (1) hyperactivation of molecules, such as EGFR, Her-2 and ras, that lie along growth factor pathways; (2) hyperactivation of molecules, such as hdm-2, that limit p53 expression levels; (3) hypoactivation or inactivation of pro-apoptotic factors, such as arf and apaf-1.

A common strategy for treating cancers with wild type p53, therefore, is to develop drugs that will reverse the effects of the various means cancer cells use to limit p53 production. This would make it more likely that agents that induce p53, such as many chemotherapeutic agents and ionizing radiation, would more likely induce p53-dependent cell death in such cancer cells. It is to be noted that p53-dependent programmed cell death is a common but not the only means whereby p53 can prevent damaged cells from proliferating. For example, p53 also functions in inducing senescence or autophagy in target cells. It is to be understood that when p53-dependent programmed cell death is mentioned herein that this is only the most common p53-dependent mechanism for irreversibly preventing the proliferation of cells with the types of damage that induce p53 and that other p53-dependent means for inhibiting the proliferation of defective cells such as senescence or autophagy may be occurring instead of programmed cell death.

In contrast to the inhibition of the death pathway, p53-dependent cell cycle arrest and repair function is largely retained in cancer cells with wild-type p53. When wild-type p53 function is inhibited in cancer cells in conjunction with the use of a genome-damaging agent, (such as conventional chemotherapy radiation or other oxidizing agents which damage DNA), cell cycle arrest occurs and DNA-repair is blocked. Consequently, p53-independent programmed cell death is triggered as a result of the replication of the damaged DNA (Waldman et al. (1996) Nature 381:713-716; Wang (1996) J Natl Cancer Inst 88: 956-965; Sak (2003) Cancer Gene Ther 10: 926-934.

In contrast to cancer cells, normal cells are more apt to undergo p53-dependent programmed cell death rather than p53-dependent cell cycle arrest and DNA repair because they have not developed the protective mechanisms of cancer cells that inhibit programmed cell death. Further, since normal cells do not have hyperactive growth factor pathways, they are not driven to copy their damaged DNA. Accordingly, normal cells are less likely to trigger p53-independent programmed cell death when p53 is inhibited, consequently they survive, and once the inhibition of p53 function is over, p53-dependent repair can occur (Pritchard et al., (1998) Cancer Res 58: 5453-5465; Wlodarski et al., (1998) Blood 91: 2998-3006; Komarov et al., (1999) Science 285: 1733-1737; Botchkarev et al., (2000) Cancer Res 60: 5002-5006).

As a result, inhibition of wild type or mutated p53 in cancer cells can sensitize them to the induction of programmed cell death while the same p53 inhibition in normal cells can protect them from the induction of p53-dependent programmed cell death. In cancer therapeutic terms, the p53 inhibitors of the present invention can sensitize cancers to many different anti-cancer agents while protecting normal cells from the toxic effects of such agents. Further, agents that damage cells and induce p53 dependent programmed cell death are generated as part of numerous medical disorders where they contribute substantially to the associated morbidity and mortality. These disorders include ischemia reperfusion injury, numbers neurodegenerative diseases aneurism etc. (Table 1 includes many more examples). When p53 mutates in a malignant stem cell, the cell loses its ability to induce p53-dependent cycle arrest and DNA repair. The loss of these repair functions sets up a powerful selection pressure that limits the allowed p53 mutations to those that provide gains-of-function that include increasing the threshold for p53-independent programmed cell death induction. A common category of such gain-of-function properties involves mutant p53 functioning as a transcriptional regulator that stimulates one or more growth factor pathways, thereby increasing resistance to programmed cell death and promoting proliferation. Thus, when mutant p53 is inhibited these gain-of-function properties are inhibited and the cancer is rendered much more likely to undergo p53-independent programmed cell death. (Girnita et al., (2000) Cancer Res 60: 5278-5283).

Numerous abnormalities in translation related processes can cause up-regulation of p53 which, in turn, is associated with subsequent programmed cell death and/or cell cycle arrest in affected cells. By inhibiting cellular proliferation, p53 can provide cells time to correct imbalances related to translation related components that would be exaggerated should proliferation continue. One such type of abnormality involves imbalances in the relative amounts of ribosomal proteins. Examples of such ribosomopathies include, for example Diamond Blackfan anemia, Shwachman-Diamond syndrome and del(5q) MDS (Narla A and Elbert B L (2010) Blood 115: 3196-3205). These disorders commonly involve bone marrow failure and in particular refractory anemia. Ribosomopathies may involve inactivating mutations in one of the two alleles for certain ribosomal proteins leading to haploinsufficiency of the involved protein. The resulting imbalance in ribosomal components up-regulates p53 by interfering with its degradation. This up-regulation is associated with programmed cell death or cell cycle arrest affecting hematopoietic progenitors leading to anemia and, in some instances, to a more generalized bone marrow failure.

In Treacher Collins syndrome, the abnormality associated with translation may involve mutations in genes encoding subunits of RNA polymerases I and III (Dauwerse J G et al., (2011) Nature Genetics 43: 20-22). Diminished function of these enzymes can result in various types of translation related abnormalities including a deficiency in ribosomal RNA or in transfer RNA.

In model systems of ribosomopathies, inhibiting p53 expression can reverse the observed adverse effects (Barlow J L et al., (2010) Nature Med 16: 59-66; McGowan K A et al., (2008) Nature Genetics 40: 963-970; Danilova N et al., (2008) Blood 15: 5228-5237; Ebert B L et al., (2008) Nature 451: 335-339. Such inhibition has been produced using either genetic engineering or pharmacologic means.

Recently, another p53 role in stress responses has been uncovered that involves negative feedback loops between p53 and certain hormone receptors, such as the estrogen, androgen and glucocorticoid receptors (Ganguli G et al., (2002) EMBO Rep 3: 569-574; Sengupta S and Wasylyk B (2004) Ann NY Acad Sci 1024: 54-71. Glucocorticoids, for example, promote erythropoiesis in stress situations such as hypoxia. The up-regulation of p53 related to a variety of pathologic conditions, (e.g., translation abnormalities, carcinogenesis, genomic damage) is associated with inhibitory effects on the actions of these hormones causing or exacerbating the underlying medical disorder. In the case of refractory anemia, antagonism of the pro-erythroid effects of the glucocorticoid hormones by up-regulated p53 contributes to the anemia.

Stress responses sometimes involve the mobilization of stem cells. p53 has recently been found to be involved in maintaining normal stem cell (cells capable of self-renewal or producing progenitor cells) and progenitor cell (cells incapable of self-renewal but retain the capacity to differentiate into a large number of cells of a particular type)quiescence. This has been most thoroughly studied in the case of hematopoietic stem cells and progenitors (Liu Y et al., (2009) Cell Stem Cell 4: 37-48; Leonova K I et al., (2010) Cell Cycle 9: 1434-1443). Inhibition of p53 in vitro or in vivo, therefore, results in an increase in the proportion of these cells that are in cycle and in an expansion in their absolute numbers along with increasing the numbers of mature myeloid and erythroid cells.

SGP2 (also referred to in the art as TRPM-2, Apo J, clusterin or CLU) is an alternatively spliced protein that is expressed in cells in multiple forms in different cell types. SGP2 is expressed by most cells in the body and may be secreted or localized to the, nuclear or cytoplasmic compartments. Extracellular functions of SGP2 include interactions with growth factor pathways and such interactions can be associated with inhibition of apoptosis. High expression of secreted or cytoplasmic SGP2 in cancer cells provides an anti-apoptotic function as seen in increase resistance to anticancer agents (Rizzi F and Bettuzzi S (2010) Endocrine Related Cancer 17: RT1-R17).

Alternative splicing of SGP2 pre-mRNA is subject to differential regulation by various cellular processes that can be altered in diseased cells. For example, patterns of expression are typically altered in cancer cells such that expression levels of the anti-apoptotic variants are increased relative to the apoptotic variants.

Two homologs (CLI and SP-40,40) are also produced by the SGP2 gene. These are distinguished by substantial divergence in the 5′ untranslated sequence. Both of these homologs bind to complement components and inhibit complement mediated cellular lysis and are of importance in biological processes such as reproduction.

### SUMMARY OF THE INVENTION

In accordance with the present invention, a composition comprising at least one agent that inhibits p53 expression is provided. An exemplary agent comprises at least one oligo sequence which hybridizes to a p53 encoding nucleic acid in a biologically acceptable carrier wherein said oligo has a composition corresponding to that depicted in FIG. 1 and numbered 2-33 and which has an RNase H mechanism of action or in Table 2. In another embodiment, the composition comprises at least two agents which rely on different cellular mechanisms to inhibit p53 expression, in a biologically acceptable carrier. In one such related embodiment said oligo has a composition corresponding to the sequences shown in Table 2 where one oligo is preferably selected from the primary translational start site group and at least one is selected from the secondary translational start site group. In an alternative related embodiment one such oligo is selected from those in FIG. 1 and at least one oligo is selected from those in Table 2. The oligos in FIG. 1 (other than 17, 18, 34-39 and the phosphodiester control which is oligo 1) having an intended RNase H dependent mechanism of action and those in Table 2 have a steric hindrance mechanism of action. The use of oligos provided herein from both classes to treat the same patient can have greater effectiveness than using compounds from only one class. In any of the compounds in FIG. 1 or Table 2, the composition may further comprise a carrier that facilitates cellular uptake and/or directs the oligo to a particular tissue or tissues.

Also provided is a method for inhibiting p53 expression in a cell or tissue comprising: contacting said cells or tissue with an effective amount of at least one agent as described above which inhibits p53 expression under conditions whereby said agent enters said cells and reduces p53 expression relative to untreated cells. Such agents are effective to modulate cellular programming and other p53-dependent activities, for example, apoptosis or cell cycle arrest in said cell. The aforementioned method is useful for the treatment or prevention of the disorders listed in Table 1.

Further, the steric hindrance oligos shown in Table 2 that inhibit the primary translational start site can be used to inhibit p53-dependent functions that require the N-terminus encoded by p53 mRNA that is initiated by the primary translational start site but which is missing from the truncated p53 that is initiated at the secondary translational start site. This approach, for example, can be used to retain many of the anticancer effects of inhibiting wild type p53 such as sensitizing the cancer to ionizing radiation or chemotherapy that induces wild type p53 activity such as cell cycle arrest and damage repair while retaining important tumor suppressor functions. This strategy can also be applied to the non-cancer medical indications listed in Table 1, for example the treatment of refractory anemia, in order to generate a truncated p53 that can have a tumor suppressor function. In the case of cancer with mutated p53 the suppression of the generation of additional full-length p53 production in tumors by a primary start site steric hindrance oligo in Table 2 while generating the truncated version can produce an added anticancer effect since the truncated protein can interfere with the cancer promoting effects of the residual mutant p53.

In addition, the use of a p53 inhibitor or combination of the p53 inhibitors shown in FIG. 1 and/or in Table 2 (where the inhibitor(s) chosen from Table 2 includes an oligo that binds the primary translational start site) in combination with Go6976 in order to achieve an enhanced anticancer effect particularly when also used with chemotherapy or radiation, FIGS. 3, 4 and 5. This enhancement provided by Go6976, a compound whose activities include the inhibition of certain forms of protein kinase C, can also be used to reduce the dose of chemotherapy or radiation to reduce the toxicity to normal cells.

p53 antisense oligos are also provided that surprisingly cause p53 levels in cells to rise rather than to fall, FIG. 2. These compounds (depicted in FIG. 1 and numbered 17, 18 and 34-39 can be used, for example, to increase wild type p53 levels in those cancer cells that express it and thereby overcome the mechanisms the developing cancer put in place during its evolution that restrict p53 to relatively low levels. In this way p53-dependent programmed cell death, or other p53 anticancer effects such as senescence or autophagy, can be promoted in these cancers with a concomitant therapeutic effect. Further the use of these p53 enhancing oligos can be used to achieve therapeutic effects in normal cells such as protecting them from chemotherapy or radiation by actions such as taking them out of cycle.

In accordance with the present invention, a composition comprising at least one agent that inhibits clusterin expression is provided. An exemplary agent comprises an oligo sequence which hybridizes to a clusterin encoding nucleic acid in a biologically acceptable carrier wherein said oligo has a composition corresponding to those listed in Tables 4A, 4B and 4C as well as Tables 5 through 9. In another embodiment, the composition comprises at least two agents that inhibit clusterin expression, in a biologically acceptable carrier. In one such related embodiment said oligo has a composition corresponding to the sequences shown in Tables 5 through 9 where one oligo is preferably selected from the primary translational start site group and at least one is selected from the secondary translational start site group. In an alternative related embodiment one such oligo is selected from those in Tables 4A, 4B and 4C and at least one oligo is selected from those in Tables 5 through 9. The oligos in Tables 4A, 4B and 4C having an RNase H dependent mechanism of action and those in Tables 5 through 9 have a steric hindrance mechanism of action. The use of oligos provided herein from both classes to treat the same patient can have greater effectiveness than using compounds from only one class. In any of the compounds in Tables 4 through 9, the composition may further comprise a carrier that facilitates cellular uptake and/or directs the oligo to a particular tissue or tissues.

The commercial applications of the clusterin antisense oligos provided above are listed in Table 3.

### BRIEF DESCRIPTION OF THE DRAWINGS

FIG. 1 provides the sequence and modification information for a series p53 directed oligos which are designed to be dependent upon RNAse H function to produce p53 suppressor activity (oligos 2-33). Surprisingly, oligos 17, 18 and 34-39 stimulate p53 expression.

FIG. 2 is a set of histograms illustrating the ability of the oligos tested to modulate p53 production. Two oligos that do not support RNase H activity (oligos 17 and 18) were found to increase p53 levels.

FIG. 3 is a series of graphs showing the results obtained with H460 cells treated with PMO (3 uM, 2 h), Dox (200 ng/ml, 24 h) followed by Go6976 (100 nM, 24 h).

FIG. 4 is a graph showing the results obtained with U2OS cells treated with PMO (3 uM, 2 h), Dox (200 ng/ml, 24 h) followed by Go6976 (100 nM, 24 h).

FIG. 5 is a graph showing the results obtained with U2OS cells treated with PMO (3 uM, 2 h), Cis (5 uM, 24 h) followed by Go6976 (100 nM, 24 h).

### DETAILED DESCRIPTION OF THE INVENTION

As described herein, the roles of wild type and mutant p53 in protecting cancer, and the presence of properly regulated p53-dependent programmed cell death induction in normal cells provide the conditions necessary for p53 inhibitors to sensitize cancers to many standard as well as novel cancer treatments while protecting normal tissues. In addition, the ability of the p53 inhibitors described to restrain p53-dependent programmed cell death and other p53-dependent functions will find application to other medical disorders where p53 plays a major role in promoting the morbidity of the disorder as well as in other commercial applications (Table 1).

Most chemotherapeutic drugs and ionizing radiation preferentially attack dividing cells. At best these treatments have a narrow therapeutic index in that they are marginally more toxic to cancer cells than they are to normal cells. Often the cancer, however, is too resistant to the treatment resulting in unacceptable toxicity to normal cells with unacceptable normal cell death as a common outcome. Constitutively self-renewing normal tissues such as gastrointestinal epithelium, bone marrow and hair follicles are generally most affected. Adding a p53 inhibitor to these treatments provides the potential for increasing the therapeutic index such that the cancer becomes substantially more sensitive to genome damaging treatments while normal cells are protected.

The design and implementation of optimized strategies involving p53 inhibitors to treat cancer should take a number of factors into consideration beyond those just described. These can include the organization of specific cancers into stem cells, proliferating non-stem cells and end-stage cells as well as identifying therapeutic targets in molecular pathways that modulate the following: (1) control of cell proliferation particularly in stem cells; (2) the induction of cell cycle checkpoints; (3) regulation of the balance between cell cycle arrest & repair and programmed cell death in response to genomic damage; (4) gain-of-function properties of particular p53 mutants.

Acute myeloid leukemia (AML) blast populations freshly obtained from patients were the first cancer cell population to be shown to consist of a very small proportion of stem cells, a larger population of proliferating non-stem cells (their proliferative potential is increasingly restricted with each division) and a predominant population of proliferatively inert end stage cells, despite the morphologic appearance of being a uniform population of immature blood cells. This cancer serves as a model for the cellular organization and interactions of other cancers that can be applied to the design of more effective therapies.

Importantly and like many other cell types, the growth rate and viability of AML blast populations are dependent, to varying degrees, on the density of the population. Further the level of the density dependence of the AML blasts is inversely correlated with prognosis that is the less density dependent populations have a worse prognosis. The effect of this variable density dependence likely explains why a spectrum of responses occurs in response to chemotherapy and why, at least in part, some patients fail to respond to such treatment. Specifically the lower the density dependence of the tumor for growth the lower the effectiveness of treatments that primarily work by reducing tumor mass (debulking the tumor).

Stem cells are the most difficult subpopulation of cancer cells to attack with cytoreductive therapy for two reasons. First, they are substantially more resistant to programmed cell death induction (or to other anti-proliferative programs) when compared to more mature cancer cells that manifest a greater sensitivity to chemotherapy (Costello et al., (2000) Cancer Res 60: 4403-4411).

Some of these mechanisms involve hyperactivation of growth factor pathways that promote a reduction in density dependent growth (i.e. the reduction in the cancer cell density required to promote proliferation). Second, a subset of cancer stem cells are not in cycle at any given time, affording them added resistance to chemotherapy. In addition, the proportion of a cancer population that consists of stem cells is positively correlated with poor prognosis (van Rhenen (2005) Clin Cancer Res 11: 6520-6527.

The high resistance of AML stem cells to chemotherapy is likely the reason there is a positive correlation between the intensity of induction chemotherapy and outcome (Buchner (2001) Cancer Chemother Pharmacol 48: Suppl 1: S41-S44).

This pattern of cellular organization and elevated stem cell resistance to conventional cancer therapy described for AML is being increasingly extended to cancer more generally (Huff C A, et al., (2006). Blood 15: 431-434; Iannolo G, et al., (2008) Critical Rev Oncol/Hemat 66: 42-51; Wang J C Y (2007). Cell Stem Cell 1: 497-501). These concepts have important implications for treatments based on p53 suppression including the selection of combinations of therapeutic agents and in selecting superior regimens for their administration.

It follows from the foregoing, that it is particularly important to kill cancer stem cells. p53 suppressors can contribute to this in several ways. In cancers with wild type p53, for example, p53 suppressors used in combination with genome-damaging agents can be used to trigger p53-independent programmed cell death as described previously. An important trigger of this program involves the facilitation of the amplification of such damage by a p53 suppressor in cancer cells capable of replicating their DNA. Thus, the p53 suppressor is potentially capable of synergizing with one or more genome damaging agents to overcome the numerous molecular mechanisms cancer stem cells have for resisting the induction of programmed cell death by genomic damaging agents. The cancer stem cells, however, preferably must not be in Go at the time of treatment and the cell cycle checkpoints preferably must not be operational in the cancer stem cells because replication of the damaged DNA can trigger a form of p53-independent programmed cell death. p53 suppressors by themselves may be sufficient to inhibit cell cycle checkpoints such as would otherwise occur as a result of treating cancer cells with a genome damaging agent or they may need to be supplemented by a second agent such as Go6976 (Kohn E A et al., (2003) Cancer Res 63: 31-35). Further, cancer stem cells can be treated with agents, such as promyelocytic leukaemia protein (PML) inhibitors that can put them into cycle (Bernardi R, Morotti A et al. (2008) Nature 453: 1072-1078).

Current strategies for treating AML rely on reducing the mass of the tumor below the threshold required for efficient growth of the AML stem cells. If the growth rate is sufficiently slowed then remissions can occur. In contrast, refractoriness to chemotherapy likely results from a relatively reduced density-dependence for growth such that remissions are not possible. Not surprisingly, such patients have been shown to have a higher proportion of AML stem cells in cycle at any given time compared to patients who respond to treatment. Intrinsic drug resistance is a general attribute of AML stem cells and they are not all in cycle at any given time, necessitating the use of high dose chemotherapy. The requirement for high-dose chemotherapy dictates infrequent treatment due to toxicity that requires significant time for recovery. This also provides time for recovery, if not expansion, of the AML stem cell pool.

The mechanism of action of p53 suppressors and the cellular organization of cancers suggests a different strategy for treating cancers that centers on depleting the size of the cancer stem cell pool over time by more frequent treatments with p53 suppressors in combination with at least low dose anticancer treatment. In the case of cancers with wild type p53 at least one agent in the anticancer treatment should include a genome-damaging agent such as chemotherapy or radiation. These agents induce p53-dependent cell cycle arrest and damage repair.

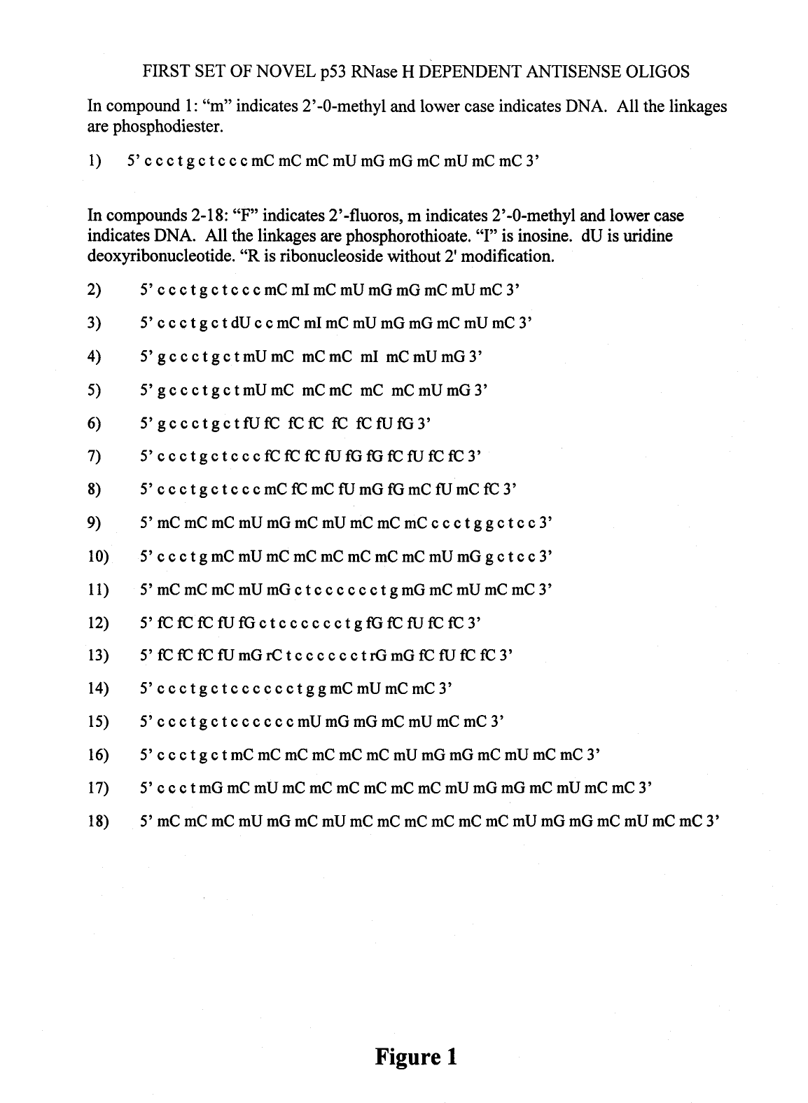

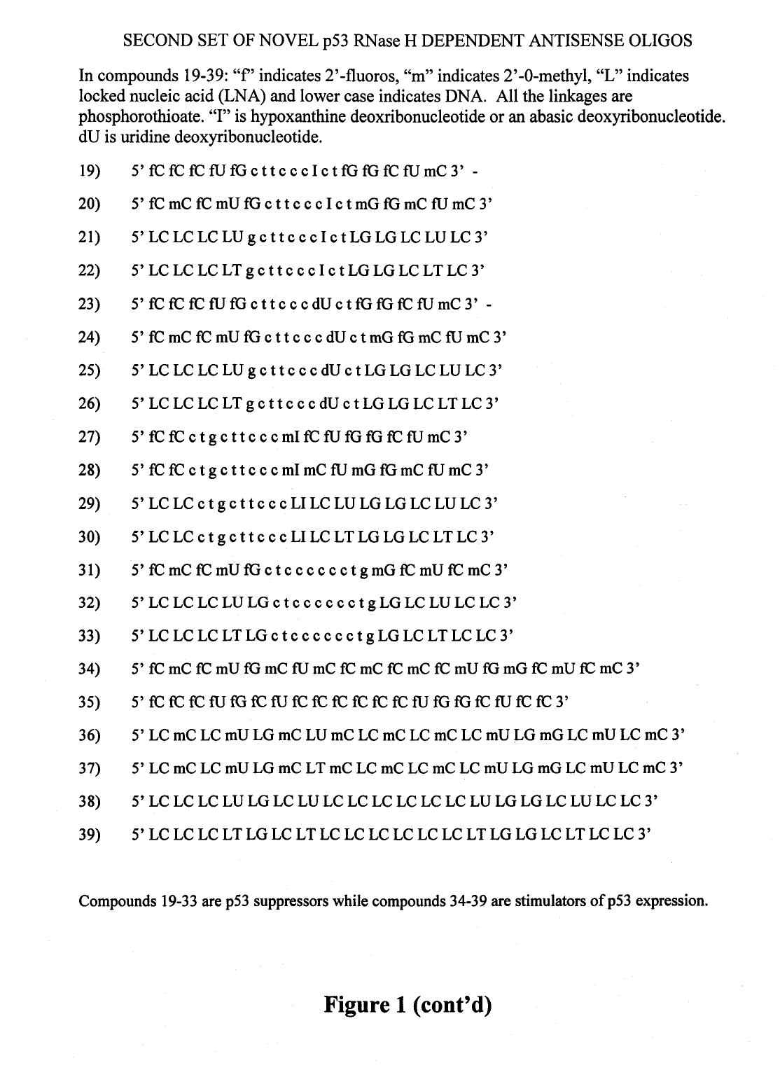

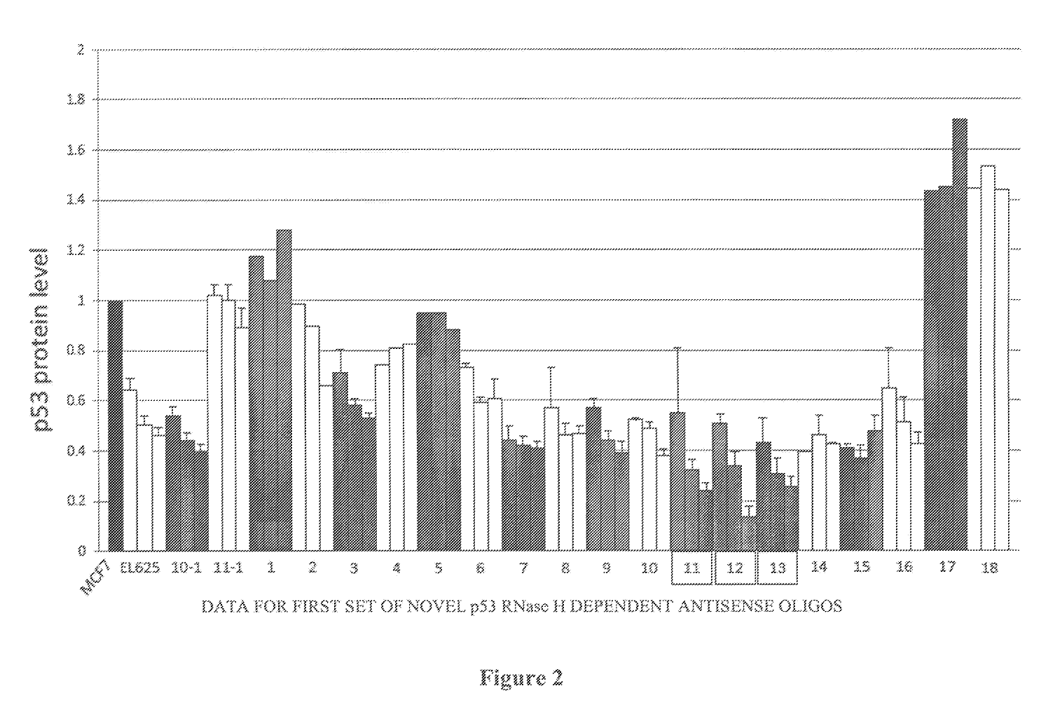

The most active RNase H dependent p53 antisense oligos shown in FIG. 1 in terms of p53 suppression (FIG. 2) are best suited to achieve an anticancer effect in patients particularly when used in combination with one or more of the p53 steric hindrance oligos provided in Table 2. In addition to the chemical modifications shown in FIG. 1 the sequences shown may be modified as follows: the 5′ and 3′ 2-3 terminal nucleosides are LNA the center is 4 or 5 deoxyribose nucleosides and the rest are FANA. Many cell types do not have sufficient RNase H activity levels to adequately support RNase H dependent antisense oligos. Further, RNase H levels can vary the same cells as a function of various conditions, such as proliferative status, with the effect that the support for RNase H dependent antisense oligos can vary from poor to good. One cell group that routinely has sufficient activity levels comprises proliferating stem cells. In the case of many cancers, such as AML, the end stage cells, unlike the stem cells do not have sufficient levels of RNase H while the proliferating non-stem cells typically have intermediate levels. RNase H dependent oligos are better suited for suppressing the expression of specific genes in proliferating stem cells than are steric hindrance antisense oligos because they are catalytic. On the other hand steric hindrance oligos work independently of RNase H status. Thus, disorders, such as cancer, that involve constitutively self-renewing tissues can be most effectively treated by using a combination of RNase H dependent and steric hindrance p53 antisense oligos. The former efficiently work in the proliferating stem cells while the latter more efficiently work in cells with insufficient RNase H levels. (These same principles are also true for the use of RNase H and steric hindrance clusterin antisense oligos.)

Some examples of treatment regiments for the use of both oligos types in the same cancer patent include the following: (1) In situations where tumor debulking is not immediately required, repeatedly treat with just the RNase H depended p53 inhibitor compound typically in combination with other anticancer treatments to drive down the cancer stem cells as they come into cycle followed by treatment with a steric hindrance p53 oligo as needed to put residual cancer stem cells in cycle and/or to debulk the cancer. By not debulking the cancer immediately the greater tumor mass may promote the proliferation of the cell density dependent malignant stem cells; or (2) In situations where tumor debulking is immediately required, treat with the RNase H and the steric hindrance p53 oligos in combination along with other anticancer agents and then follow with repeated treatments with at least the RNase H dependent p53 oligo as needed. In this way the treatment regimen can be tailored to particular patients.

Such tailoring can be further refined in instances where cancers can be readily biopsied or otherwise sampled. Here the RNase H activity involved in supporting RNase H dependent antisense oligos can be monitored as a means to determine the effectiveness of therapy that includes RNase H dependent anticancer agents such as those disclosed herein as well as how frequently to treat a patient with active disease with such a regimen. This approach can also be applied more generally to monitoring anticancer treatments by using high RNase H measurements (compared to the levels in non-proliferating cancer cells preferably from the same patient or normal cells such as peripheral blood mononuclear cells) as a surrogate marker for the proliferating cancer stem cells. Further, monitoring peripheral blood mononuclear cell isolates (which have low levels of the appropriate RNase H activity compared to proliferating solid cancer stem cells or hematologic cancer stem cells) obtained by the standard density gradient technique for the appropriate RNase H activity can used to do either of the following: (1) monitor the effectiveness of a RNase H dependent anticancer agent containing regimen and (2) predict a relapse hence when to resume treatment. The RNase H activity level preferably is determined by measuring the ability of cell lysates to degrade RNA/DNA duplexes where the RNA is labeled in a manner that can be assayed with high activity levels reflecting the presence of proliferating cancer stem cells. In the case of tumor containing samples the appropriate RNase H activity level and approximate frequency of proliferating cancer stem cells also can be approximated using an antibody for RNase H1 in an immunohistochemical or other antibody based assay. These approaches can also be applied to other appropriate body fluids where proliferating solid cancer stem cells or hematologic cancer stem cells may appear such as ascites in the case of ovarian cancer.

Significant morbidity associated with certain medical disorders is mediated to a substantial degree by p53-dependent programmed cell death and other p53-dependent anti-proliferative programs such as autophagy and senescence. Accordingly the p53 suppressors disclosed herein will be of therapeutic use for the treatment of such disorders.

One group of such disorders involves the toxic effects of conventional cancer therapies including chemotherapy and radiation. Specific toxicities involving p53-dependent programmed cell death include but are not limited to damage to constitutively self renewing tissues such as bone marrow, gastrointestinal epithelium, skin (including hair follicles), the lining of the oral cavity and throat. These toxicities tend to occur with any cancer treatment that preferentially targets proliferating cells. Other toxicities involving p53-dependent programmed cell death include but are not limited to veno-occlusive disease, capillary leak syndrome, peripheral nerve damage, hearing loss, kidney damage and impairment of cognitive function (Chemo-brain). These toxicities tend to preferentially occur with smaller subsets of cancer treatments. For example, cisplatin (and other platinum based chemotherapeutic agents) tend to cause kidney damage, peripheral nerve damage and hearing loss. Biologics based on IL-2 tend to cause capillary leak syndrome is another example. The following references provide more detail on the association of particular toxic effect with particular cancer treatments and are incorporated herein by reference: (1) Physicians' Desk Reference 2008 62nd edition Thompson Healthcare Brooklyn, N.Y.; (2) Cancer: Principles & Practice of Oncology 2008 8th edition V. T. DeVita et al. editors, Lippincott, Williams and Wilkins Philadelphia Pa.; (3) Cancer Medicine 2006 7th edition D. W. Kufe editor BC Decker Inc. Hamilton, Ontario Canada; and (4) Cancer Chemotherapy & Biotherapy 2005 4th edition B. A. Chabner and D. L. Longo editors Lippincott, Williams and Wilkins Philadelphia Pa.

Another group of disorders in which p53-dependent (including but not limited to p53-dependent programmed cell death, autophagy, senescence and cell cycle arrest) adverse effects significantly impact morbidity and are, therefore, disorders where p53 suppressors will provide therapeutic benefit include but are not limited to the following: (1) heart failure such as is caused by but which is not limited to prolonged cardiac hypertrophy (Sano M, Minamino T, Toko H et al. (2007). Nature 446: 444-448); (2) septic cardiomyopathy Buerke U, Carter J M, Schlitt A et al. (2008). Shock 29: 497-503) (3) ischemia-reperfusion injuries including but not limited to those associated with organ transplant, including but not limited to heart, kidney, liver and lungs; treatment or prophylaxis of myocardial infarction; treatment or prophylaxis of stroke (Hu Y, Zou Y, Hala M et al. (2000). Prolonged survival of heart allografts from p53-deficient mice. Transplantation 69: 2634-2640; Zebrowski D C, Alcendor R R, Kirshenbaum L A et al. (2006). Caspase-3 mediated cleavage of MEKK1 promotes p53 transcriptional activity. J Mol Cell Cardiology 40: 605-618; Venkatapuram S, Wang C, Krolikowski J G et al. (2006). Inhibition of apoptotic protein p53 lowers the threshold of isoflurane-induced cardioprotection during early reperfusion in rabbits. Anesth Analg 103: 1400-1405; Liu P, Baohuan X, Cavalieri T A et al. (2006). Pifithrin-alpha (a small molecule inhibitor of wild type p53) attenuates p53-mediated apoptosis and improves cardiac function in response to myocardial ischemia/reperfusion in aged rats. Shock 26: 608-614; Kelly K J, Plotkin Z, Vulgamott S L et al. (2003). p53 mediates the apoptotic response to GTP depletion after renal ischemia-reperfusion: protective role of a p53 inhibitor. J Am Soc Nephrol 14: 128-138; Leker R R, Aharonowiz M, Grieg N H et al. (2004). The role of p53-induced apoptosis in cerebral ischemia: effects pifthrin-alpha. Exp. Neurol 187: 478-486; (4) fatty liver disease including but not limited to the associated liver injury (Yahagi N, Shimano H, Matsuzaka T et al. (2004). J Biol Chem 279: 20571-20575); (5) Huntington's Disease (Bae B, Xu H, Igarashi S et al. (2005). Neuron 47: 29-41); (6) cerebral vasospasm (Zhou C, Yamaguchi M, Colohan A L et al. (2005). J Cerebral Blood Flow & Metabol 25: 572-582) Further descriptions of some examples of p53 related medical conditions are provided below. The first group deals with disorders related to cancer treatments. Examples of disorders not related to cancer are in the second group. A more extensive listing of commercial uses of p53 inhibitors is provided in Table I.

### Gastrointestinal Toxicity

Gastrointestinal toxicity is one of the most common adverse sequelae related to various aspects of tissue transplantation including but not limited to conditioning regimens, graft vs. host disease, and the treatment of graft vs. host disease and/or chemotherapy administration. It can involve the entire gastrointestinal tract or be more localized (Pritchard et al., (1998) Cancer Res 58: 5453-5465). It is usually managed symptomatically with narcotics and topical anesthetics. A novel keratinocyte growth factor, palifermin, reduces the incidence of oral mucositis in adults.

### Neurocognitive and Neuropsychological Effects of Chemotherapy (Chemo Brain Syndrome)

A low intelligence quotient (IQ), sleep disorders, fatigue, memory problems, and developmental delays have all been reported in chemotherapy recipients. Chemotherapy administration can also compromise functioning of peripheral nerves and impact vision and hearing. These issues, which are mediated to a substantial degree by p53-dependent programmed cell death, must be mitigated to improve the person's overall quality of life.

### Capillary Leak Syndrome

Systemic capillary leak syndrome (CLS) or Clarkson's disease is a rare medical condition where the number and size of the pores in the capillaries are increased which leads to a leakage of fluid from the blood to the interstitial fluid, resulting in dangerously low blood pressure (hypotension). An important mechanism underlying CLS is the p53-dependent programmed death of vascular cells. CLS is characterized by weight gain, ascites, edema and multi-organ dysfunction, including non-cardiogenic pulmonary edema with or without pleural effusion. CLS can occur as an idiopathic disorder (ICLS) and/or as a systemic syndrome (SCLS). Often, it has been described in patients who have elevated levels of certain hematopoietic growth factors that have direct or indirect effects on the vasculature that produce the leaking. These growth factors include but are not limited to G-CSF, VEGF and IL-2. CLS is a well known side effect of high dose IL-2 therapy such as is used for the treatment of certain solid tumors. It has also been described following immunosuppressive therapy (Funke et al., Ann Hematol 68: 49-52, 1994). Further, the syndrome has been found in BMT recipients at the time of stem cell infusion or hematopoietic recovery (Cahill R A, Spitzer T R, Mazumder A. Marrow engraftment and clinical manifestations of capillary leak syndrome. Bone Marrow Transplant 1996; 18: 177-184), and in breast carcinoma and lymphoma patients recovering from high-dose therapy followed by G-CSF administration with or without PBPC support (Busmanis I A, Beaty A E, Basser R L. Isolated pleural effusion with hematopoietic cells of mixed lineage in a patient receiving granulocyte-colony-stimulating factor after high-dose chemotherapy. Diagn Cytopathol 1998; 18: 204-207; Oeda E, Shinohara K, Kamei S et al.

### Hair Loss

Chemotherapy drugs are powerful medications that attack rapidly growing cancer cells. Unfortunately, these drugs also attack other rapidly growing cells in your body—including those present in hair roots. Hair loss may occur all over the body—not just on the scalp. In certain cases, eyelashes, eyebrows, armpit, pubic and other body hair also falls out. Some chemotherapy drugs are more likely than others to cause hair loss, and different doses can cause anything from a mere thinning to complete baldness. Botchkarev V., et al., (2000) Cancer Res 60: 5002-5006) describe the essential role p53 plays in this process. Thus, administration of oligos which down modulate p53 expression should be effective to ameliorate and reduce hair loss associated with chemotherapy administration.

### Nephrotoxicity

Chemotherapy, such as cisplatin, carboplatin, carmustine, methotrexate, can cause nephrotoxicity, and renal impairment can result in altered excretion and metabolism of chemotherapeutic agents, resulting in increased systemic toxicity. In addition biologic therapy such as IL-2 or interferon-alpha can cause renal toxicity as can certain other drugs including but not limited to amphotericin B, gentamycin, vancomycin and ACE inhibitors.

A variety of renal disease and electrolyte disorders can result from the treatment of malignant disease. Chemotherapeutic agents can affect the glomerulus, tubules, interstitium or the renal microvasculature, with clinical manifestations that range from an asymptomatic elevation of serum creatinine to acute renal failure requiring dialysis. The kidneys are one of the major elimination pathways for many antineoplastic drugs and their metabolites, further enhancing their potential for nephrotoxicity.

Delayed drug excretion can result in increased systemic toxicity and is a major concern in patients with renal impairment. Many drugs require dose adjustment when administered in the setting of renal insufficiency. It has been reported that inhibition of p53 expression can attenuate ischemic and cisplatin-induced acute kidney injury. Accordingly, the oligonucleotides targeted to p53 described herein should also be effective for these purposes (Molitoris B., et al. (2007) Am J Physiol Renal Physiol. (2007) 293(4): F1282-91,

### Toxicity to the Hematopoietic System

Cytotoxic chemotherapy is often complicated by hematopoietic toxicity. The degree of aplasia and the rapidity of count recovery following chemotherapy are indicative of bone marrow reserve. Patients who generally have a normal bone marrow function will recover from chemotherapy-induced cytopenia relatively rapidly. In contrast, patients that have poor bone marrow reserve will have significantly prolonged period of aplasia. Wlodarski et al. (1998) Blood 91: 2998-3006) described the modulatory role of p53 in this process. Thus, agents that down modulate p53 expression such as the oligos described herein should prove efficacious in facilitating hematopoietic recovery following exposure to cytotoxic agents.

There are numerous other medical conditions aside from cancer and the treatment of malignancy that will benefit from the administration of agents that modulate p53 dependent cell death. These are described below and in the Examples.

### Ischemia Reperfusion Injury

Ischemic heart disease is a common age-related disease. Apoptotic cell death and inflammation are the major contributors to ischemia reperfusion injury. The mechanisms that trigger myocyte apoptosis and inflammation during myocardial ischemic reperfusion remain to be elucidated however it is clear that up-regulation of p53 and resultant p53 dependent cell death play a role.

The small intestine is also highly sensitive to ischemia-reperfusion (I/R) induced injury that is associated with high morbidity and mortality. Apoptosis, or programmed cell death, is a major mode of cell death occurring during I/R induced injury. However, the mechanisms by which I/R cause apoptosis in the small intestine are poorly understood. p53 up-regulated modulator of apoptosis (PUMA) is a p53 downstream target and a member of the BH3-only group of Bcl-2 family proteins. It has been shown that PUMA plays an essential role in apoptosis induced by a variety of stimuli in different tissues through a mitochondrial pathway.

### Neurological Disease

Alzheimer's disease (AD) and Parkinson's disease (PD) are two of the most significant neurodegenerative disorders in the developed world. However, although these diseases were described almost a century ago, the molecular mechanisms that lead to the neuronal cell death associated with these diseases are not yet clear, and vigorous research efforts have failed to identify effective treatment options. A role for mitochondria in the release of proapoptotic proteins, such as cytochrome c and apoptosis-inducing factor (AIF) etc., is evident along with induction of key processes involving oxidative stress and activation of glutamate receptors. Data imply that DNA damage resulting in p53 induction and reentry in the cell cycle is also involved in neurodegeneration. Thus, p53 also provides a valuable target for the prevention of neurological disease.

### Skin Disorders

DNA damage by UV radiation plays an essential role in skin cancer induction. It appears that even sub-erythemal doses of solar simulating radiation, are capable of inducing substantial nuclear damage, namely pyrimidine dimers and p53 induction in human skin in situ. The quantity and distribution of p53 induced in human skin by UV radiation depended highly on the waveband and dose of UV used. Solar simulating radiation induced very high levels of p53 throughout all layers in epidermal keratinocytes 24 hr following an erythemal dose (230+/−15.9/1000 cells), and the induction followed a dose response. Following UVA I+II and UVA I radiations, p53 expression was approximately half of that seen with equivalent biological doses of solar simulating radiation (63.5+/−28.5 and 103+/−15.9, respectively). Expression of p53 was seen in basal cell keratinocytes at lower doses of UVA, but all layers of the epidermis were affected at higher doses.

The oligonucleotides described herein are useful for modulating the function of target molecules that regulate cellular proliferation and cell death. Administration of such oligos for the treatment and management of a variety of medical conditions is encompassed by the present invention.

The following definitions are provided to facilitate an understanding of the present invention.

“Conventional antisense oligos” are single stranded oligos that inhibit the expression of the targeted gene by one or both of the following mechanisms: (1) steric hindrance—e.g., the antisense oligo interferes with some step in the sequence of events leading to gene expression resulting in protein production by directly interfering with the step. For example, the antisense oligo may bind to a region of the RNA transcript of the gene that includes a start site for translation which is most often an AUG sequence (other possibilities are GUG, UUG, CUG, AUA, ACG and CUG) and as a result of such binding the initiation of translation is inhibited; and (2) induction of enzymatic digestion of the RNA transcripts of the targeted gene where the involved enzyme is not Argonaute 2.

“RNase H” is most often the enzyme involved in antisense oligo directed mRNA degradation. There are multiple RNase H enzymes in mammalian cells and not all support this class of oligos. RNase H 1 provides this capability as do certain other RNase H enzymes that are not well characterized. RNase H 2 is comparatively unimportant in this context. (The RNase H designations used here are based on the harmonization of the mammalian RNase H nomenclature with the prokaryote nomenclature). RNase H recognizes DNA/RNA or certain DNA analog/RNA duplexes (not all oligos that are DNA analogs will support RNase H activity) and digests the RNA adjacent to the DNA or DNA analog hybridized to it.

“Nucleotides” or “nucleosides” for convenience, the monomers comprising the oligo sequences of individual oligos will be termed herein “nucleotides” or “nucleosides” but it is to be understood that the normal sugar moiety (deoxyribose or ribose) and/or the normal base (adenine, guanine, thymine, cytosine and uracil) moieties may be substantially modified or even replaced by functionally similar analogs. For example, the normal sugar may have a fluorine inserted in the 2′ position or be entirely replaced by a different ring structure as is the case with piperazine or morpholino oligos. Further, in particular embodiments, the nucleotides or nucleosides within an oligo sequence may be abasic. In addition, the linkers between the monomers will often be varied from the normal phosphodiester structure and can include one or more of several other possibilities depending on such considerations as the need for nuclease resistance, high target sequence binding affinity, pharmacokinetics and preferential uptake by particular cell types. The alternating linker/sugar or linker/sugar substitute structure comprising oligos are referred to as the “backbone” while the normal bases or their substitutes occur as appendages to the backbone.

“Cell penetrating peptides” (CPPs) are peptides that promote cell penetration. CPPs may be naturally occurring protein domains or they may be designed based on the naturally occurring versions. CPPs typically share a high density of basic charges and are approximately 10-30 amino acids in length (J Immun Meth 325: 114-126; Wright et al. (2003) Curr Protein Peptide Sci 4: 105-124. Also see U.S. Pat. Nos. 7,169,814; 6,759,387; 6,669,951; 6,593,292; 6,495,663; 6,306,993; and 7,585,834, as well as US applications 2007/0173436; 2002/0009491; 2004/0186045; 2002/0127198; and 2003/0032593, to Rothbard et al. The preferred linkers for binding CPPs to oligos are described in Moulton et al., (2004) Bioconj Chem 15: 290-299 with the most preferred being KMUS and sulfo-LCSPDP. CPPs useful in the oligos of the invention are described further herein below. Particularly preferred CPPs for the present invention include (RX)₈B and R₆Pen where R is arginine, X is 6-aminohexanoic acid, B is beta-alanine and Pen is the peptide Penetratin with the amino acid sequence RQLKIWFQNRRMKWKK. One of these can be attached to either the 5′ or the 3′ ends of the oligo.

“Peptoids” are a type of peptide mimic that have been developed as antimicrobial agents, synthetic lung surfactants and as ligands for certain proteins such as VEGF. More recently peptoids have been developed as alternatives to CPPs (Wender et al., (2000) Proc Nat Acad Sci 97: 13003-13008; Wright et al. (2003) Curr Protein Peptide Sci 4: 105-124). Particularly preferred peptoids for use in the invention include, without limitation, peptoids such as those described in U.S. Pat. No. 7,169,814 as well as in US patent applications 2004/0186045; 2002/0009491

“Endosomolytic and lysosomotropic agents” are agents that can be used in combination with an oligo to promote the release of said oligo from endosomes, lysosomes or phagosomes. The former are agents that are attached to oligos or incorporated into particular oligo delivery systems while the latter agents may be so attached or incorporated or be administered as separate agents from, but in conjunction with, any such oligo used with or without a delivery system. Lysosomotropic agents have other desirable properties and can exhibit antimicrobial activity. In addition, oligos that inhibit wild type p53 expression can interfere with endosome, lysosome and phagosome production and function thereby reducing oligo sequestration in these structures. This reduction surprisingly improves bioavailability and, therefore, enhances the inhibitory activity of oligos that are administered during the time p53 expression is suppressed.

An endosomal lytic moiety refers to an agent that possesses at least endosomal lytic activity. In certain embodiments, an endosomal lytic moiety also exhibits lysosomolytic, phagosomolytic or lysosmotropic activity.

A “specific binding pair” comprises a specific binding member and a binding partner that have a particular specificity for each other and which in normal conditions bind to each other in preference to other molecules. Such members and binding partners are also referred to as targeting molecules herein. Examples of specific binding pairs include but are not limited to ligands and receptor, antigens and antibodies, and complementary nucleic acid molecules. The skilled person is aware of many other examples. Further the term “specific binding pair” is also applicable to where either or both of the specific binding pair member and the binding partner comprise a part of a larger molecule.

A “cellular program” refers to the appearance in cells, of a cell-type restricted coordinated pattern of gene expression over time. The fundamental or overarching program is a “differentiation program” that produces the basic differentiated phenotype of the cell, for example, producing a liver cell or a blood cell of a particular type, and that such differentiated phenotypes in turn determine the responses, if any, of the cell in question to exogenous or endogenous cues, for example DNA damage resulting from exposure to chemotherapy or radiation. These responses include cellular programs that control cellular viability and proliferation. Thus the differentiation program is a master program that controls various secondary programs.

A “stem cell” is a rare cell type in the body that exhibits a capacity for self-renewal. Specifically when a stem cell divides the resulting daughter cells are either committed to undergoing a particular differentiation program (along with any progeny) or they are a replica of the parent cell. In other words, the replica cells are not committed to undergo a differentiation program. When the division of a stem cell produces daughter cells that are replicas of the parent cell, the division is called “self-renewal.” Accordingly, stem cells are able to function as the cellular source material for the maintenance and/or expansion of a particular tissue or cell type.

There are many types of stem cells and often any given type exists in a hierarchy with respect to the differentiation potential of any daughter cells committed to undergoing a differentiation program. For example, a more primitive hematopoietic stem cell could have the capacity to produce committed daughter cells that in turn have the capacity to give rise to progeny that include any myelopoietic cell type while a less primitive hematopoietic stem cell might be only capable of producing committed daughter cells that can give rise to monocytes and granulocytes.

“Embryonic stem (ES) cells” are stem cells derived from embryos or fetal tissue and are known to be capable of producing daughter cells that are duplicates of the parent ES or that differentiate into cells committed to the production of cells and tissues of one of the three primary germ layers.

“Induced pluripotent (iPS) stem cells” are created (induced) from somatic cells by human manipulation. Such manipulation has typically involved the use of expression vectors to cause the expression of certain genes in the somatic cells. “Pluripotent” refers to the fact that such stem cells can produce daughter cells committed to one of several possible differentiation programs.

“Chemotherapeutic agents” are compounds that exhibit anticancer activity and/or are detrimental to a cell by causing damage to critical cellular components, particularly the genome (e.g., by causing strand breaks or other modifications to DNA). In anti-cancer applications, it may be desirable to combine administration of the oligos described herein with administration of chemotherapeutic agents, radiation or biologics. Suitable chemotherapeutic agents for this purpose include, but are not limited to: alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitrosoureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)).

The phrase “p53-dependent cell death” or “p53-dependent apoptosis” includes a variety of different cellular processes that lead to an irreversible stoppage of cell proliferation most often by cell death. In some instances the actual mechanism may involve apoptosis, p53-dendendent senescence or some other p53-dependent death pathway such as autophagy.

“Introducing into” means uptake or absorption in the cell, as is understood by those skilled in the art. Absorption or uptake of nucleic acid or small molecules can occur through cellular processes, or by auxiliary agents or devices. For example, for in vivo delivery, nucleic acids can be injected into a tissue site or administered systemically. In vitro delivery includes methods known in the art such as electroporation and lipofection.

As used herein and as known in the art, the term “identity” is the relationship between two or more polynucleotide sequences, as determined by comparing the sequences. Identity also means the degree of sequence relatedness between polynucleotide sequences, as determined by the match between strings of such sequences. Identity can be readily calculated (see, e.g., Computation Molecular Biology, Lesk, A. M., eds., Oxford University Press, New York (1998), and Biocomputing: Informatics and Genome Projects, Smith, D. W., ed., Academic Press, New York (1993), both of which are incorporated by reference herein). While there exist a number of methods to measure identity between two polynucleotide sequences, the term is well known to skilled artisans (see, e.g., Sequence Analysis in Molecular Biology, von Heinje, G., Academic Press (1987); and Sequence Analysis Primer, Gribskov., M. and Devereux, J., eds., M. Stockton Press, New York (1991)). Methods commonly employed to determine identity between sequences include, for example, those disclosed in Carillo, H., and Lipman, D., SIAM J. Applied Math. (1988) 48:1073. “Substantially identical,” as used herein, means there is a very high degree of homology (preferably 100% sequence identity) between the sense strand of the dsRNA and the corresponding part of the target 3′-UTR of the viral genome. However, dsRNA having greater than 90%, or 95% sequence identity may be used in the present invention, and thus sequence variations that might be expected due to genetic mutation, strain polymorphism, or evolutionary divergence can be tolerated. Although 100% identity is preferred, the dsRNA may contain single or multiple base-pair random mismatches between the RNA and the target 3′-UTR.

As used herein, the term “treatment” refers to the application or administration of a therapeutic agent to a patient, or application or administration of a therapeutic agent to an isolated tissue or cell line from a patient, who has a disorder, e.g., a disease or condition, a symptom of disease, or a predisposition toward a disease, with the purpose to cure, heal, alleviate, relieve, alter, remedy, ameliorate, improve, or affect the disease, the symptoms of disease, or the predisposition toward disease.

As used herein, a “pharmaceutical composition” comprises a pharmacologically effective amount of a nucleic acid or small molecule and a pharmaceutically acceptable carrier. As used herein, “pharmacologically effective amount,” “therapeutically effective amount” or simply “effective amount” refers to that amount of an agent effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter.

The term “pharmaceutically acceptable carrier” refers to a carrier or diluent for administration of a therapeutic agent. Pharmaceutically acceptable carriers for therapeutic use are well known in the pharmaceutical art, and are described, for example, in Remington's Pharmaceutical Sciences, Mack Publishing Co. (A. R. Gennaro, ed. 1985), which is hereby incorporated by reference herein. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

As used herein, a “transformed cell” is a cell into which a nucleic acid molecule has been introduced by means of recombinant DNA techniques.

Oligo backbone configurations that demonstrate particularly high binding affinities to the target (measured by melting temperature or Tm) are preferred for implementing the steric hindrance mechanism. LNA, FANA, 2′-fluoro, morpholino and piperazine containing backbones are particularly well suited for this purpose.

Morpholino oligos are commercially available from Gene Tools LLC. Morpholino oligo characteristics and synthesis include but are not limited to those presented in the following: Summerton and Weller, Antisense Nucleic Acid Drug Dev 7: 187, 1997; Summerton, Biochim Biophys Acta 1489: 141, 1999; Iversen, Curr Opin Mol Ther 3: 235, 2001; U.S. Pat. No. 6,784,291, U.S. Pat. No. 5,185,444, U.S. Pat. No. 5,378,841, U.S. Pat. No. 5,405,938, U.S. Pat. No. 5,034,506, U.S. Pat. No. 5,142,047, U.S. Pat. No. 5,235,033. Morpholino oligos for the purposes of the present invention may have the uncharged and/or at least one cationic linkages between the nucleoside analogs made up of a morpholino ring and a normal base (guanine, uracil, thymine, cytosine or adenine) or a unnatural base as described herein. The preferred linkage for morpholino oligos is phosphorodiamidate which is an uncharged linkage. In some embodiments it may be modified as discussed below to provide a positive charge.

In one embodiment, the morpholino subunit has the following structure:

### Schematic of a Morpholino Subunit

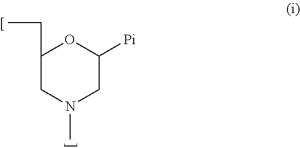

where Pi is a base-pairing moiety, and the linkages depicted above connect the nitrogen atom of (i) to the 5′ carbon of an adjacent subunit. The base-pairing moieties Pi may be the same or different, and are generally designed to provide a sequence which binds to a target nucleic acid.

The use of embodiments of linkage types (b1), (b2) and (b3) above to link morpholino subunits may be illustrated graphically as follows:

### Schematic of Linkages for Morpholino Subunit

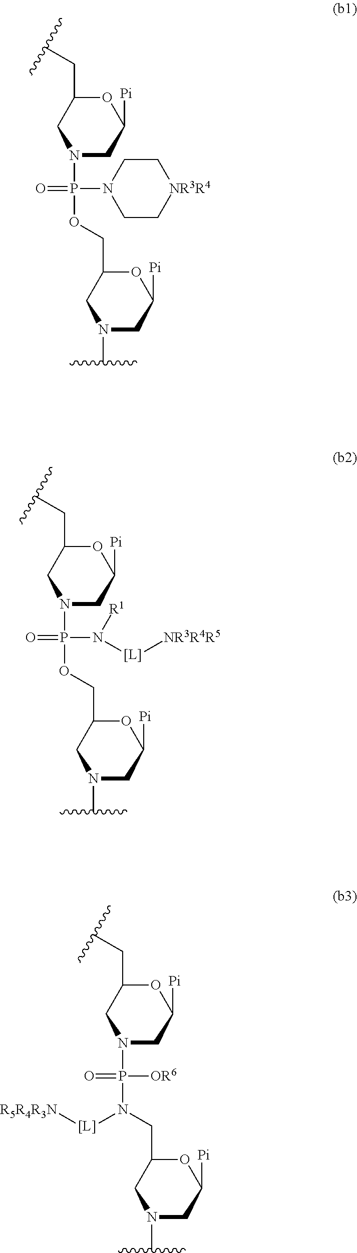

Preferably, at least 5% of the linkages in an oligo are selected from cationic linkages (b1), (b2), and (b3); in further embodiments, 10% to 35% of the linkages are selected from cationic linkages (b1), (b2), and (b3). As noted above, all of the cationic linkages in an oligo are preferably of the same type or structure.

In further embodiments, the cationic linkages are selected from linkages (b1′) and (b1″) as shown below, where (b1″) is referred to herein as a “Pip” linkage and (b1″) is referred to herein as a “GuX” linkage:

In the structures above, W is S or O, and is preferably O; each of R1 and R2 is independently selected from hydrogen and lower alkyl, and is preferably methyl; and A represents hydrogen or a non-interfering substituent on one or more carbon atoms in (b1′) and (b1″). Preferably, each A is hydrogen; that is, the nitrogen heterocycle is preferably unsubstituted. In further embodiments, at least 10% of the linkages are of type (b1′) or (b1″); for example, 20% to 80%, 20% to 50%, or 20% to 30% of the linkages may be of type (b1′) or (b1″). In other embodiments, the oligo contains no linkages of type (b1). Alternatively, the oligo contains no linkages of type (b1) where each R is H, R³ is H or CH₃, and R⁴ is H, CH₃, or an electron pair.

In still further embodiments, the cationic linkages are of type (b2), where L is a linker up to 12 atoms in length having bonds selected from alkyl (e.g. —CH₂—CH₂—), alkoxy and alkylamino (e.g. —CH₂—NH—), with the proviso that the terminal atoms in L (e.g., those adjacent to carbonyl or nitrogen) are carbon atoms.

The morpholino subunits may also be linked by non-phosphorus-based intersubunit linkages, as described further below, where at least one linkage is modified with a pendant cationic group as described above. For example, a 5′ nitrogen atom on a morpholino ring could be employed in a sulfamide linkage or a urea linkage (where phosphorus is replaced with carbon or sulfur, respectively) and modified in a manner analogous to the 5′-nitrogen atom in structure (b3) above.

The subject oligo may also be conjugated to a peptide transport moiety which is effective to enhance transport of the oligo into cells. The transport moiety is preferably attached to a terminus of the oligo.

### Schematic of Attachment of a Cell Penetrating Peptide to Morpholino Backbone

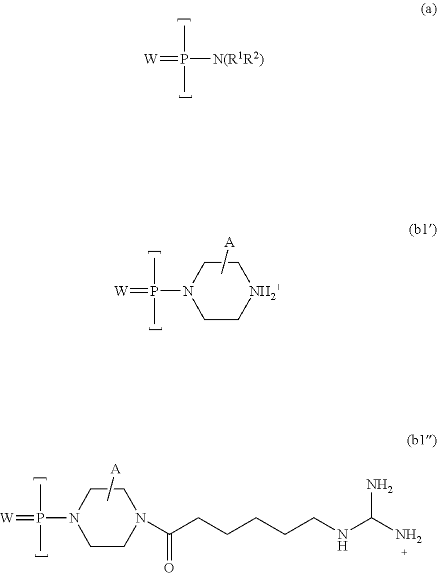

In the structures above, W is S or O, and is preferably O; each of R¹ and R² is independently selected from hydrogen and lower alkyl, and is preferably methyl; and A represents hydrogen or a non-interfering substituent on one or more carbon atoms in (b1′) and (b1″). Preferably, each A is hydrogen; that is, the nitrogen heterocycle is preferably unsubstituted. In further embodiments, at least 10% of the linkages are of type (b1′) or (b1″); for example, 20% to 80%, 20% to 50%, or 20% to 30% of the linkages may be of type (R) or (b1″). In other embodiments, the oligo contains no linkages of type (b1′). Alternatively, the oligo contains no linkages of type (b1) where each R is H, R³ is H or CH₃, and R⁴ is H, CH₃, or an electron pair.

In still further embodiments, the cationic linkages are of type (b2), where L is a linker up to 12 atoms in length having bonds selected from alkyl (e.g. —CH₂—CH₂—), alkoxy (—C—O—), and alkylamino (e.g. —CH₂—NH—), with the proviso that the terminal atoms in L (e.g., those adjacent to carbonyl or nitrogen) are carbon atoms.

The morpholino subunits may also be linked by non-phosphorus-based intersubunit linkages, as described further below, where at least one linkage is modified with a pendant cationic group as described above. For example, a 5′nitrogen atom on a morpholino ring could be employed in a sulfamide linkage or a urea linkage (where phosphorus is replaced with carbon or sulfur, respectively) and modified in a manner analogous to the 5′-nitrogen atom in structure (b3) above.

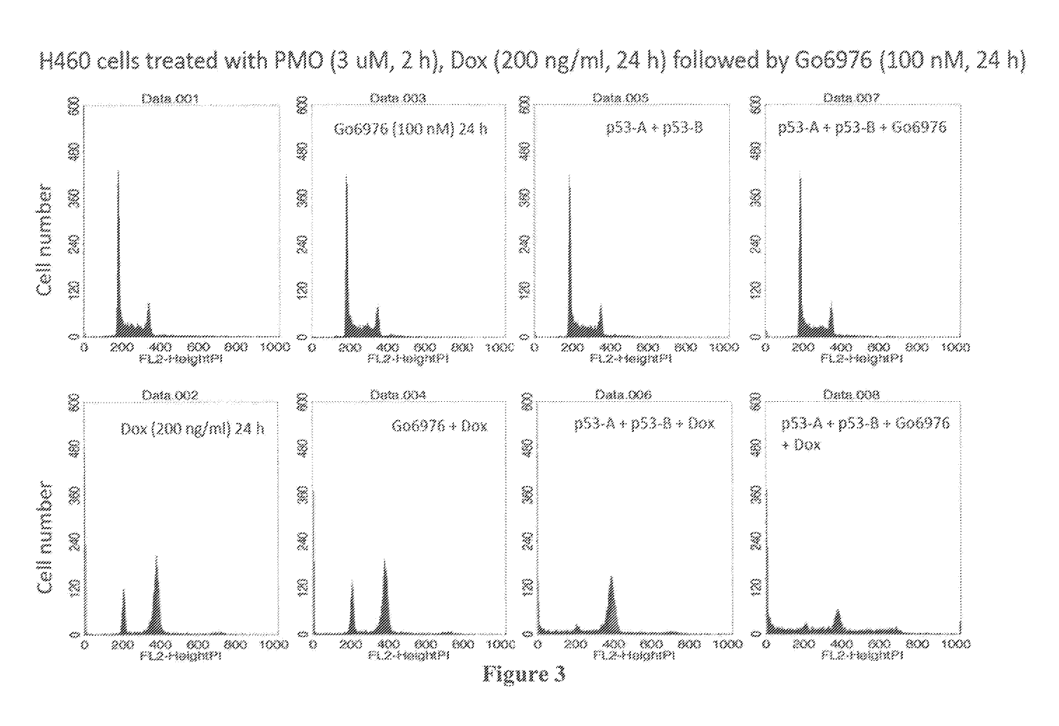

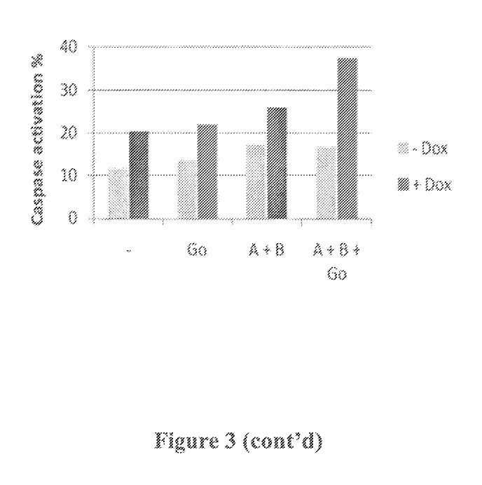

The subject oligo may also be conjugated to a peptide transport moiety that is effective to enhance transport of the oligo into cells. The transport moiety discussed further herein below and is preferably attached to a terminus of the oligo, as shown, for example, in FIG. 3.

Also preferred are oligos that comprise a piperazine ring in the place of the ring ribose or deoxyribose sugar. Such analogs are described in U.S. Pat. No. 6,841,675 to Schmidt et al. Methods for synthesizing piperazine based nucleic acid analogs are also disclosed in the '675 patent. Such substitutions improve in vivo bioavailability and exhibit lower aggregation characteristics. The amino acid-derived side chain functionality denoted R² and R³ in the formula below is unique. This region of the molecule provides useful biological and medicinal applications beyond antisense nucleobase/nucleobase interactions and hydrogen bonding. In some embodiments of the instant invention, nucleoside analogs represented by the following formula are included:

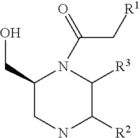

The formula shows the schematic representation of this embodiment with R¹ selected from the group consisting of adenine, thymine, uracil, guanine and cystosine. R² and R³ are side chain groups derived from amino acids and amino acid analogs, or any diastereoisomeric combinations thereof. As such, R² and R³ may be selected from the group consisting of hydrogen and/or all side chains occurring in the 20 natural amino acids in all isomeric and diastereoisomeric forms and derivatives thereof, such as, but not limited to Serine=CH₂OH, and Lys=(CH₂)₄NH₂. In other embodiments, the nucleobase is a nucleobase derivative selected from the group consisting of inosine, fluorouracil, and allyluracil. The nucleobase may further be chosen from a group of nucleobase analogs including daunamycin, and other polycyclic or aromatic hydrocarbon residues known to bind to DNA/RNA.

In many of these embodiments, the piperazine nucleic acid analogs may be so configured as to be capable of forming a phosphoramidite, sulfonamide, phosphorodiamidate, phosphorodiamidate modified to have a positive charge as described for certain morpholino oligos or carbonylamide backbone linkage. They may also generally be rapidly assembled in a few synthetic steps from commercial grade materials. The length of the linkage between piperazine rings in the oligo of the instant invention may vary from one to four carbons in length, and may be branched or unbranched. The oligos of the instant invention are also compatible with standard solid phase synthesizers, and may thus be used with synthesizers currently used in the art to allow easy assembly of molecules containing them.

The invention further comprises amide-, phosphonamide-, carbamate-, and sulphonamide-linked oligos made up of homo-oligonucleotides or comprising a chimera of either DNA or RNA and the nucleoside analogs of the instant invention. In some embodiments, the oligo is a composition containing a number, n, of nucleoside monomers represented by the formula:

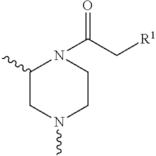

wherein R¹ is a nucleobase selected from the group consisting of adenine, thymine, uracil, guanine, and cytosine; wherein n is from about 1 to about 30; and wherein the nucleoside monomers are joined by amide-, phosphonamide-, carbamate-, or sulfonamide-linkages. In some of these embodiments, R¹ may be a nucleobase derivative selected from the group consisting of inosine, fluorouracil, and allyl uracil. In others, the nucleobase derivative is chosen from a group including daunamycin and other polycyclic or aromatic hydrocarbon residues known to bind to DNA/RNA. In some of these oligonucleotide compositions n is from about 1 to about 30. The invention further includes oligos containing branching from the side chains of the amino acids, rings of oligos and other tertiary, non-linear structures.

As previously noted, in some of these oligonucleotide compositions, phosphodiester linkages join the monomers. In some of these, the phosphodiester bonds comprise a linker of between about 1 and about 4 carbons in length. In others the monomers are joined by peptide bonds. In some of these, the peptide bonds comprise a linker of between about 1 and about 4 carbons in length. Finally, in other embodiments, sulfonamide bonds join the monomers. In some of these, the sulfonamide bonds comprise a linker of between about 1 and about 4 carbons in length. In other embodiments, carbamate linkages join the monomers. In some of these, the carbamate bonds consist of a linker of between 1 to 4 carbons in length. Included are also all possible chimeric linkages of the possible structures.

Since the steric hindrance mechanism is not dependent on RNase H activity, oligos using this mechanism have the potential to be active in cells where RNase H levels are too low to adequately support conventional antisense oligo effects dependent on this mechanism. Stem cells an early progenitor cells have adequate levels of RNase H for this purpose while cells that have differentiated beyond the stem or progenitor cell stage typically do not. When functional, however, oligos that support the RNase H based mechanism have the potential advantage over steric hindrance based mechanism of working catalytically since the same oligo molecule is capable of inactivating numerous target RNA molecules. As discussed elsewhere herein it is also possible to modify LNA, FANA, 2′-fluoro, morpholino and piperazine containing backbones to enable or increase their potential to catalyze the cleavage of their target RNA by RNase H by inserting certain linkers, acyclic nucleosides or by using the gapmer approach.

The availability of antisense oligos directed to the inhibition of the same target gene by different or overlapping inhibitory mechanisms allows for greater flexibility in treatment options for certain medical disorders. In cancer, for example, RNase H dependent oligos can be used to attack the malignant stem and progenitor cells while sparing other cells in the cancer. If the success of the treatment requires the malignant stem and progenitor cells to be in cycle there can be an advantage to not attacking the other cells in the cancer because they can promote the proliferation of the malignant stem and progenitor cells. In other instances, rapidly debulking the tumor mass in a patient may be important. Here an antisense oligo with a steric hindrance mechanism would be the agent of choice since it will be operative on a much broader range of cancer cells. If the antisense oligo is intended to protect normal tissues from the toxic effects of conventional cytotoxic cancer therapeutics, then one with a combined RNase H and steric hindrance mechanism may be preferred so that the range of normal cell types is more broadly and thoroughly protected.

For use in kits and diagnostics, the nucleic acids and small molecules of the present invention, either alone or in combination with other compounds or therapeutics, can be used as tools in differential and/or combinatorial analyses to elucidate expression patterns of a portion or the entire complement of genes expressed within cells and tissues.

Expression patterns within cells or tissues treated with one or more antisense compounds are compared to control cells or tissues not treated with antisense compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression)(Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-1904) and mass spectrometry methods (reviewed in (To, Comb. Chem. High Throughput Screen, 2000, 3, 235-41).

The specificity and sensitivity of nucleic acid based therapies is also harnessed by those of skill in the art for therapeutic uses. Antisense oligonucleotides have been employed as therapeutic moieties in the treatment of disease states in animals and man. Antisense oligonucleotide drugs, including ribozymes, have been safely and effectively administered to humans and numerous clinical trials are presently underway. It is thus established that oligonucleotides can be useful therapeutic modalities that can be configured to be useful in treatment regimes for treatment of cells, tissues and animals, especially humans.

In the context of this invention, the term “oligonucleotide” refers to an oligomer or polymer of ribonucleic acid (RNA) or deoxyribonucleic acid (DNA) or mimetic thereof which can be single or double stranded. This term includes oligonucleotides composed of naturally-occurring nucleobases, sugars and covalent internucleoside (backbone) linkages as well as oligos having non-naturally-occurring portions which function similarly. Such modified or substituted oligos are often preferred over native forms because of desirable properties such as, for example, enhanced cellular uptake, enhanced affinity for nucleic acid target and increased stability in the presence of nucleases.

Chimeric antisense compounds of the invention may be formed as composite structures of two or more oligo chemistries such as combining RNA or RNA analogs with DNA or DNA analogs. Representative United States patents that teach the preparation of such structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The nucleic acid based therapeutics used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The nucleic acid based therapeutics and small molecules of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

For example, where oligonucleotide nucleic acid is to be transcribed into RNA, the nucleic acid may be operatively linked to a suitable promoter element, for example, but not limited to, the cytomegalovirus immediate early promoter, the Rous sarcoma virus long terminal repeat promoter, the human elongation factor 1α promoter, the human ubiquitin c promoter, etc. It may be desirable, in certain embodiments of the invention, to use an inducible promoter. Non-limiting examples of inducible promoters include the murine mammary tumor virus promoter (inducible with dexamethasone); commercially available tetracycline-responsive or ecdysone-inducible promoters, etc. In specific non-limiting embodiments of the invention, the promoter may be selectively active in cancer cells; one example of such a promoter is the PEG-3 promoter, as described in International Patent Application No. PCT/US99/07199, Publication No. WO 99/49898 (published in English on Oct. 7, 1999); other non-limiting examples include the prostate specific antigen gene promoter (O'Keefe et al., 2000, Prostate 45:149-157), the kallikrein 2 gene promoter (Xie et al., 2001, Human Gene Ther. 12:549-561), the human alpha-fetoprotein gene promoter (Ido et al., 1995, Cancer Res. 55:3105-3109), the c-erbB-2 gene promoter (Takalcuwa et al., 1997, Jpn. J. Cancer Res. 88:166-175), the human carcinoembryonic antigen gene promoter (Lan et al., 1996, Gastroenterol. 111:1241-1251), the gastrin-releasing peptide gene promoter (Inase et al., 2000, Int. J. Cancer 85:716-719). the human telomerase reverse transcriptase gene promoter (Pan and Koenman, 1999, Med. Hypotheses 53:130-135), the hexokinase II gene promoter (Katabi et al., 1999, Human Gene Ther. 10:155-164), the L-plastin gene promoter (Peng et al., 2001, Cancer Res. 61:4405-4413), the neuron-specific enolase gene promoter (Tanaka et al., 2001, Anticancer Res. 21:291-294), the midkine gene promoter (Adachi et al., 2000, Cancer Res. 60:4305-4310), the human mucin gene MUC1 promoter (Stackhouse et al., 1999, Cancer Gene Ther. 6:209-219), and the human mucin gene MUC4 promoter (Genbank Accession No. AF241535), which is particularly active in pancreatic cancer cells (Perrais et al., 2001, J. Biol Chem. 17; 276(33):30923-33).

Suitable expression vectors include virus-based vectors and non-virus based DNA or RNA delivery systems. Examples of appropriate virus-based gene transfer vectors include, but are not limited to, those derived from retroviruses, for example Moloney murine leukemia-virus based vectors such as LX, LNSX, LNCX or LXSN (Miller and Rosman, 1989, Biotechniques 7:980-989); lentiviruses, for example human immunodeficiency virus (“HIV”), feline leukemia virus (“FIV”) or equine infectious anemia virus (“EIAV”)-based vectors (Case et al., 1999, Proc. Natl. Acad. Sci. U.S.A. 96: 22988-2993; Curran et al., 2000, Molecular Ther. 1:31-38; Olsen, 1998, Gene Ther. 5:1481-1487; U.S. Pat. Nos. 6,255,071 and 6,025,192); adenoviruses (Zhang, 1999, Cancer Gene Ther. 6(2):113-138; Connelly, 1999, Curr. Opin. Mol. Ther. 1(5):565-572; Stratford-Perricaudet, 1990, Human Gene Ther. 1:241-256; Rosenfeld, 1991, Science 252:431-434; Wang et al., 1991, Adv. Exp. Med. Biol. 309:61-66; Jaffe et al., 1992, Nat. Gen. 1:372-378; Quantin et al., 1992, Proc. Natl. Acad. Sci. U.S.A. 89:2581-2584; Rosenfeld et al., 1992, Cell 68:143-155; Mastrangeli et al., 1993, J. Clin. Invest. 91:225-234; Ragot et al., 1993, Nature 361:647-650; Hayaski et al., 1994, J. Biol. Chem. 269:23872-23875; Bett et al., 1994, Proc. Nati. Acad. Sci. U.S.A. 91:8802-8806), for example Ad5/CMV-based E1-deleted vectors (Li et al., 1993, Human Gene Ther. 4:403-409); adeno-associated viruses, for example pSub201-based AAV2-derived vectors (Walsh et al., 1992, Proc. Natl. Acad. Sci. U.S.A. 89:7257-7261); herpes simplex viruses, for example vectors based on HSV-1 (Geller and Freese, 1990, Proc. Natl. Acad. Sci. U.S.A. 87:1149-1153); baculoviruses, for example AcMNPV-based vectors (Boyce and Bucher, 1996, Proc. Natl. Acad. Sci. U.S.A. 93:2348-2352); SV40, for example SVluc (Strayer and Milano, 1996, Gene Ther. 3:581-587); Epstein-Barr viruses, for example EBV-based replicon vectors (Hambor et al., 1988, Proc. Natl. Acad. Sci. U.S.A. 85:4010-4014); alphaviruses, for example Semliki Forest virus- or Sindbis virus-based vectors (Polo et al., 1999, Proc. Natl. Acad. Sci. U.S.A. 96:4598-4603); vaccinia viruses, for example modified vaccinia virus (MVA)-based vectors (Sutter and Moss, 1992, Proc. Natl. Acad. Sci. U.S.A. 89:10847-10851) or any other class of viruses that can efficiently transduce human tumor cells and that can accommodate the nucleic acid sequences required for therapeutic efficacy.

Non-limiting examples of non-virus-based delivery systems which may be used according to the invention include, but are not limited to, so-called naked nucleic acids (Wolff et al., 1990, Science 247:1465-1468), nucleic acids encapsulated in liposomes (Nicolau et al., 1987, Methods in Enzymology 1987:157-176), nucleic acid/lipid complexes (Legendre and Szoka, 1992, Pharmaceutical Research 9:1235-1242), and nucleic acid/protein complexes (Wu and Wu, 1991, Biother. 3:87-95).

Oligonucleotides may also be produced by yeast or bacterial expression systems. For example, bacterial expression may be achieved using plasmids such as pCEP4 (Invitrogen, San Diego, Calif.), pMAMneo (Clontech, Palo Alto, Calif.; see below), pcDNA3.1 (Invitrogen, San Diego, Calif.), etc.

Depending on the expression system used, nucleic acid may be introduced by any standard technique, including transfection, transduction, electroporation, bioballistics, microinjection, etc.

### Delivery Reagents

In the present methods, the nucleic acid based molecule or oligonucleotide can be administered to the subject either as naked oligonucleotide, in conjunction with a delivery reagent, or as a recombinant plasmid or viral vector that expresses the oligonucleotide.

Suitable delivery reagents for administration in conjunction with the present oligonucleotide include the Mirus Transit TKO lipophilic reagent; lipofectin; lipofectamine; cellfectin; or polycations (e.g., polylysine), or liposomes. A preferred delivery reagent is a liposome.

Liposomes can aid in the delivery of the nucleic acid based reagents or small molecules to a particular tissue, such as retinal or tumor tissue, and can also increase the blood half-life of the antisense oligonucleotide. Liposomes suitable for use in the invention are formed from standard vesicle-forming lipids, which generally include neutral or negatively charged phospholipids and a sterol, such as cholesterol. The selection of lipids is generally guided by consideration of factors such as the desired liposome size and half-life or the liposomes in the blood-stream. A variety of methods are known for preparing liposomes, for example as described in Szoka et al. (1980), Ann. Rev. Biophys. Bioeng. 9: 467; and U.S. Pat. Nos. 4,235,871, 4,501,728, 4,837,028, and 5,019,369.

Preferably, the liposomes encapsulating the oligonucleotide or small molecule comprises a ligand molecule that can target the liposome to a particular cell or tissue at or near the site of angiogenesis for example. Ligands which bind to receptors prevalent in tumor or vascular endothelial cells, such as monoclonal antibodies that bind to tumor antigens or endothelial cell surface antigens, are preferred.

Particularly preferably, the liposomes encapsulating the nucleic acid based reagents or small molecules of the invention are modified so as to avoid clearance by the mononuclear macrophage and reticuloendothelial systems, for example by having opsonization-inhibition moieties bound to the surface of the structure. In one embodiment, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and the reticuloendothelial system (“RES”); e.g., as described in U.S. Pat. No. 4,920,016. Liposomes modified with opsonization-inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, target tissue characterized by such microvasculature defects, for example solid tumors, will efficiently accumulate these liposomes; see Gabizon, et al. (1988), P.N.A.S., USA, 18: 6949-53. In addition, the reduced uptake by the RES lowers the toxicity of stealth liposomes by preventing significant accumulation in the liver and spleen. Thus, liposomes of the invention that are modified with opsonization-inhibition moieties can deliver the nucleic acid based reagent to tumor cells.

Opsonization inhibiting moieties suitable for modifying liposomes are preferably water-soluble polymers with a number-average molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, gluconic acid, mannuronic acid, hyaluronic acid, pectic acid, neuraminic acid, alginic acid, carrageenan; aminated polysaccharides or oligosaccharides (linear or branches); or carboxylated polysaccharides or oligosaccharides, e.g., reacted with derivatives of carbonic acids with resultant linking of carboxylic groups.

Preferably, the opsonization-inhibiting moiety is a PEG, PPG, or derivatives thereof. Liposomes modified with PEG or PEG-derivatives are sometimes called “PEGylated liposomes.” See US Patent application 2008/0097087.

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture such as tetrahydrofuran and water in a 30:12 ratio at 60° C.

### Pharmaceutical Compositions

The present invention also includes pharmaceutical compositions and formulations which include the nucleic acid based reagents and small molecules of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intrathecal, intranasal, epidermal and transdermal), oral or parenteral. Parenteral administration includes intravenous, intra-arterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the oligonucleotides of the invention are in admixture with a topical delivery agent such as lipids, liposomes, fatty acids, fatty acid esters, steroids, chelating agents and surfactants. In addition to the liposomes discussed above, preferred lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). Oligonucleotides of the invention may be encapsulated within liposomes or may form complexes thereto, in particular to cationic liposomes. Alternatively, oligonucleotides may be complexed with lipids, in particular to cationic lipids. Preferred fatty acids and esters include but are not limited arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C1-10 alkyl ester (e.g., isopropylmyristate IPM), monoglyceride, diglyceride or pharmaceutically acceptable salt thereof. Topical formulations are described in detail in U.S. patent application Ser. No. 09/315,298 filed on May 20, 1999.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides of the invention are administered in conjunction with one or more penetration enhancers surfactants and chelators. Preferred surfactants include fatty acids and/or esters or salts thereof, bile acids and/or salts thereof. Preferred bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate, sodium glycodihydrofusidate. Preferred fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Oligonucleotides of the invention may be delivered orally in granular form including sprayed dried particles, or incorporated into a complex to form micro or nanoparticles. Oligonucleotide complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Particularly preferred complexing agents include chitosan, N-trimethylchitosan, poly-L-lysine, polyhistidine, polyomithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for oligonucleotides and their preparation are described in detail in U.S. patent application Ser. No. 08/886,829 (filed Jul. 1, 1997), Ser. No. 09/108,673 (filed Jul. 1, 1998), Ser. No. 09/256,515 (filed Feb. 23, 1999), Ser. No. 09/082,624 (filed May 21, 1998) and Ser. No. 09/315,298 (filed May 20, 1999).

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

### Penetration Enhancers

In one embodiment, the present invention employs various penetration enhancers to effect the efficient delivery of nucleic acids, particularly oligonucleotides, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, and non-chelating non-surfactants (Lee et al., Critical Reviews in Therapeutic Drug Carrier Systems, 1991, p. 92).

### Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The co-administration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is co-administered with polyinosinic acid, dextran sulfate, polycytidic acid or 4-acetamido-4′isothiocyano-stilbene-2,2′-disulfonic acid (Miyao et al., Antisense Res. Dev., 1995, 5, 115-121; Takakura et al., Antisense & Nucl. Acid Drug Dev., 1996, 6, 177-183).

### Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

### Other Components

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritic, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

Certain embodiments of the invention provide pharmaceutical compositions containing (a) one or more nucleic acid based therapeutics or small molecule and (b) one or more other chemotherapeutic agents which function by a non-antisense mechanism. Examples of such chemotherapeutic agents include but are not limited to daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). See, generally, The Merck Manual of Diagnosis and Therapy, 15th Ed. 1987, pp. 1206-1228, Berkow et al., eds., Rahway, N.J. When used with the compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. See, generally, The Merck Manual of Diagnosis and Therapy, 15th Ed., Berkow et al., eds., 1987, Rahway, N.J., pages 2499-2506 and 46-49, respectively). Other non-antisense chemotherapeutic agents are also within the scope of this invention. Two or more combined compounds may be used together or sequentially.

In another related embodiment, compositions of the invention may contain one or more nucleic acid based therapeutic compounds, particularly oligonucleotides, targeted to a first nucleic acid and one or more additional antisense compounds targeted to a second nucleic acid target. Numerous examples of antisense compounds are known in the art. Two or more combined compounds may be used together or sequentially.

TABLE I
Commercial Uses of Disclosed p53 Antisense Oligos

Sensitize cancers with wild type p53 to cytotoxic therapies that include but are not limited to
agents that induce p53 dependent cell cycle arrest. Such cytotoxic therapies include those
requiring administration of radiotherapy, platinum based compounds, anthracyclines,
alkylating agents, free radical generators including the use of any of the above at sub-
therapeutic or lower doses that are not cytotoxic in the absence of the p53 inhibitor;
Treat cancers with mutant p53;
Sensitize cancers with mutant p53 to the induction of apoptosis and/or autophagy and/or
other cell death or senescence inducer including where the inducer can either be a biologic
such as an antibody or a drug such as a chemotherapeutic agent or a drug that is an inhibitor
or of a molecule or family of molecules that inhibit apoptosis and/or autophagy and/or other
cell death mechanism or senescence;
Reverse stem cell and/or progenitor cell quiescence including malignant stem cells to expand
normal stem cells and progeny for various purposes such as treating one of the forms of
leucopenia or to put malignant stem cells in cycle so they can be attacked by cell cycle
dependent anti-cancer agents;
Where the p53 inhibitor is an antisense oligo with a steric hindrance mechanism of action and
the desired effect is to put quiescent stem cells or progenitor cells in cycle including where
said cells are malignant including for the purpose of rendering said cancer cells sensitive to
cell cycle dependent anti-cancer agents the anti-cancer action of which may be further
enhanced by the use of an RNase H dependent p53 antisense oligo, or p53 siRNA and/or a
different p53 steric hindrance antisense oligo;
Where the p53 inhibitor is an antisense oligo with a steric hindrance mechanism of action
where the effect is to put quiescent stem cells or progenitor cells in cycle including in order to
increase the levels of RNase H activity including but not limited to RNase H1 where the cells
are to be treated with an RNase H dependent oligo;
Treatment of heart failure;
Treatment of medical conditions where p53 dependent apoptosis and/or autophagy and/or
other cell death mechanism or senescence is promoted;
To inhibit p53-dependent apoptosis and/or autophagy and/or other cell death mechanism or
senescence in non-malignant stem cells where such mechanisms contribute to the mortality
and/or morbidity of a medical condition;
Treatment of Huntington's disease;
To treat ribosomopathies such as Diamond-Blackfan syndrome; Shwachman Diamond
Syndrome; del(5q) MDS; X-linked Dyskeratosis Congenita; Cartilage Hair Hypoplasia;
Treacher Collins Syndrome where the p53 suppressive treatment has a therapeutic effect on
the stated disorder and/or to inhibit the progression of the disorder to leukemia or another
cancer and/or to alleviate deficiencies in one or more blood cell type or platelets in the
peripheral blood;
To treat medical conditions where abnormalities related to translation are involved that may
or may not be considered ribosomopathies such as refractory cytopenia with unilineage
dysplasia (includes refractory anemia, refractory neutropenia and refractory
thrombocytopenia), refractory anemia with ring sideroblasts, refractory cytopenia with
multilineage dysplasia, refractory anemia with multilineage dysplasia, refractory anemia with
excess blasts, type 1, refractory anemia with excess blasts, type 2, refractory cytopenia of
childhood and inherited bone marrow failure syndromes such as Fanconi anemia) where the
p53 suppressive treatment has a therapeutic effect on the stated disorder and/or inhibits the
progression of the disorder to leukemia or some other cancer and/or to alleviate deficiencies
in one or more blood cell type or platelets in the peripheral blood and/or is used in
combination with a myelosuppressive chemotherapeutic agent such as an anthracycline such
as idarubicin or a topoisomerase inhibitor such as etoposide or radiotherapy or a
demethylating agent such as 5-azacytidine or 5-azadeoxycytidine to suppress the diseased
cell clone to allow normal hematopoiesis to recover or as a preparative regimen for a blood
cell transplant;
To treat Del (5q) MDS or cancer patients with Del (5q) or other MDS patients where patients
are resistant to thalidomide or a thalidomide analog such as lenalidomide either as an
alternative treatment or in combination with the thalidomide or thalidomide analog such as
lenalidomide to provide an enhanced therapeutic response including but not limited to
patients with normal or elevated activity levels of the PP2A protein phosphatase, most
particularly the c alpha variant, in their affected blood cells;
To treat Del (5q) MDS or cancer patients with Del (5q) or other MDS patients where
treatment with thalidomide or a thalidomide analog such as lenalidomide results in significant
toxicity such as one or more cytopenias and/or thrombocytopenia including but not limited to
patents with reduced activity levels of the PP2A protein phosphatase, most particularly the c
alpha variant, in their affected blood cells;
To prevent the evolution of premalignant lesions to cancer including those states
characterized by elevated levels of p53 expression and/or characterized by p53-dependent
changes in cellular programming such as p53-dependent apoptosis, autophagy, senescence
and cell cycle arrest including but not limited to the following atypical lobular hyperplasia of
the breast, atypical ductal hyperplasia of the breast, lobular carcinoma in situ of the breast,
ductal carcinoma in situ of the breast, prophylactic treatment of women with BRCA1 and/or
BRCA2 mutations, low grade and high grade intraepithelial neoplasia of the esophagus,
Barrett's esophagus, intratubular epithelial dysplasia of the kidney, Bowen's Disease,
carcinoma in situ, leukoplakia, erythroplakia, epithelial dysplasia and endometrial
hyperplasia;
Treatment of fatty liver disease;
Treatment of stress induced immunosuppression;
Treatment of sequellae associated with subarachnoid hemorrhage;
Treatment of pathologic hyperpigmentation;
Treatment of hyperkeratosis;
Treatment of toxic effects of cancer chemotherapy and radiation including but not limited to
the following: hair loss, mucositis, myelosupression, hearing loss, peripheral nerve damage,
impaired brain function and kidney damage;
Treatment of inflammatory bowel disease;
Treatment of Crohn's disease;
To improve therapeutic outcomes by promoting sensitivity to glucocorticoids (such as
prednisone, prednisolone, methylprednisolone, triamcinolone, hydrocortisone and
dexamethasone) including in disorders where glucocorticoid resistance occurs such as
refractory anemia, Crohn's Disease, ulcerative colitis, rheumatoid arthritis, and asthma as
well as in other disorders such as heart attack and inflammation and multiple organ failure
syndrome where glucocorticoids are known to provide a benefit;
Treatment of ARDS;
Treatment of multiple organ failure syndrome;
To sensitize cancers to cytotoxic treatments dependent on cell proliferation and/or DNA
replication;
To reduce amyloid deposition;
Treatment of neurodegenerative diseases;
Treatment of ischemia-reperfusion injury including that associated with organ or tissue
transplant;
To promote DNA replication in quiescent of malignant stem cells so that they become
sensitive to cycle dependent therapeutic drugs;
To increase expansion of non-malignant tissue to replace lost or damaged tissues;
To inhibit demyelination;
To treat multiple sclerosis;
To treat Alzheimer's Disease;
Treatment of Parkinson's disease;
To inhibit cell death associated with diabetic ischemia;
To inhibit spontaneous apoptosis, autophagy, other forms of programmed cell death, cell
cycle arrest, senescence and differentiation in stem cells including embryonic stem cells and
iPS such as reduces the efficiency of preparing such cells for transplantation organ generation
or the generation of animals or for use in scientific research in situations where said cells and
animals express wild type p53;
To inhibit cell death associated with cerebral ischemia;
To inhibit cell death associated with myocardial infarction including consequent heart wall
rupture;
Treatment of schizophrenia;
Treatment of psoriasis;
Treatment of AIDS;
To inhibit rupture of atherosclerotic plaques;
To prevent aneurysm rupture;
To inhibit graft vs host disease;
Treatment of systemic lupus erythematosus;
To promote healing of hard to heal wounds;
To treat capillary leak syndrome;
To inhibit the progression of emphysema;
To reduce endosomal, lysosomal or phagosomal sequestration of oligonucleotide based
therapeutics including siRNA, dicer substrates, double stranded DNA decoys for
transcriptional regulators antisense oligos and aptamers with the effect of increasing their
biologic activity;
To promote proliferation of stem cells such as hematopoietic or neural;
To treat diabetes mellitus including insulin resistant diabetes;
To treat porokeratosis;
To inhibit ferritin induced cell death such as occurs in iron overload
To promote the viability and/or promote the proliferation of cell lines used in the manufacture
of biologics

The materials and methods set forth below are provided to facilitate the practice of Example I.

Cell Culture and Transfection of Oligos into MCF7 Cells

The MCF7 breast cancer cell line was grown in α-MEM containing 10% fetal bovine serum. For transfection experiments, MCF7 cells were grown in 10-cm dishes until a confluence of about 80% was reached. oligos (obtained as lyophilized solids) were resuspended in RNase-free water at a concentration of 0.2 mM. The cells were transfected with different concentrations of the oligos (0.05, 0.2 and 0.8 μM) using Lipofectamine 2000 (Invitrogen, Ca#11668-019) as described by the manufacturer. The efficiency of transfection was measured using fluorescent labeled oligonucleotides and determined to be >90% in all experiments.

### Western Blot Analysis

Cells were lysed 24 h after transfection in SDS lysis buffer (136 mM Tris-HCl, pH 6.8, 4% w/v SDS, 20% glycerol). Protein concentration of cell lysates was measured using the Pierce BCA Protein Assay (Reagent A Ca# PI-23223, Reagent B Ca# PI-23224). The SDS lysates were supplemented with DTT (100 mM) and 0.01% bromophenol blue, heated at 99° C. for 5 min and centrifuged at 13,000 rpm for 10 min using a microcentrifuge prior to loading on polyacrylamide gels.

For Western blots visualized by enhanced chemiluminescence (ECL), cell lysates were subjected to electrophoresis on 10% SDS-polyacrylamide gels and proteins were subsequently transferred to PVDF membranes (Perkin Elmer Ca#NEF-1002). The membranes were first blocked with 5% fat-free dry milk powder in TBS (10 mM Tris-HCl, pH 7.5, 150 mM NaCl) for 1 hr and then incubated with PAb1801 monoclonal antibody against p53 (1:400 v/v after ammonium sulfate precipitation from hybridoma supernatant) for 30 min at room temperature. The membranes were then incubated with anti-ERK2 antibody (1:1000 v/v; #SC-164, Santa Cruz Biotechnology, Santa Cruz) for an additional 30 min at room temperature. After 3 washes (5 min each) with TBST (10 mM Tris-HCl, pH 7.5, 150 mM NaCl, 0.5% Tween 20), the membranes were incubated with HRP-conjugated anti-mouse IgG (1:30,000 v/v; Cedarlane Ca#515-035-003) for 30 min at room temperature. The membranes were washed 3-times with TBST (10 min each). p53 and ERK2 proteins on the membranes were visualized on X-ray film after detection with the ECL reagent according to the manufacturer's instructions (Perkin Elmer, Waltham, Mass.).

For direct fluorescence detection of p53 and ERK2 proteins on Western blots, proteins were resolved by SDS-polyacrylamide gel electrophoresis and transferred onto Immobilon-FL membranes (Fisher Ca#IPFL00010). The membranes were first incubated with 20% SuperBlock blocking buffer in TBS (Fisher Ca#PI-37535) for 1 hr at room temperature and then incubated overnight at 4° C. with PAb1801 monoclonal antibody against p53 (1:200 v/v after protein G affinity purification from hybridoma supernatant), followed by incubation with anti-ERK2 antibody for 30 min at room temperature. After 3 washes with TBST (5 min each), the blots were incubated with Cy5-conjugated goat anti-mouse IgG (Cat#115-175-146, Jackson ImmunoResearch Laboratories Inc, West Grove, Pa.) for 30 min at room temperature. After 3 washes with TBST (10 min each), the blots were air-dried for 15 min at 37° C. p53 and ERK2 proteins on the fluorescent blots were quantified on a Typhoon Trio variable mode imager.

The following examples are provided to illustrate certain embodiments of the invention. They are not intended to limit the invention in any way.

### Example I

### Inhibitors Effective for Down-Modulation of p53 Production in Target Cells

Given the medical importance of p53 regulation in the control of cellular programming including those programs involved in viability and proliferation as wells as its role in the modulation responses to various stresses, new antisense p53 oligos were synthesized which are effective to down modulate p53 expression in target cells. These are shown in FIG. 1 and Table 2 where those in the table are conjugated to (RX)₈B or R₆Pen. Each of the oligos was designed using the p53 sequence provided at GenBank accession number NM_{—}000546.4.

In the present example, a series of modified anti-p53 oligonucleotides were synthesized and their ability to modulate p53 protein production levels in target cells determined. Increasing concentration of the oligonucleotides shown in FIG. 2 (0.05, 0.2 and 0.8 μM) were transfected into MCF7 cells using Lipofectamine 2000. p53 protein levels were measured 24 hours after transfection by fluorescent Western blotting using DO1 antibodies to p53 and a secondary Cy5-conjugated anti-mouse antibody. Oligo 10-1 and 11-1 are single-stranded chimeric PS DNA/2′OMe RNAs serving as positive and non-silencing controls, respectively. Fluorescent images were quantified on a Typhoon Trio variable mode imager. Erk2 was measured in each sample and serves as a loading control. Values shown represent the ratio of p53 to Erk2 after normalization to the level of p53 in non-transfected MCF7 cells. Error bars indicate the SEM. For transfection with EL625, 10-1 and 11-1, n=9. For transfection with oligos 3, and 6-16, n=2. For transfection with oligos 1, 2, 4, 5, 17 and 18, n=1.

FIG. 2 shows that oligos numbered 11, 12 and 13 exhibit superior inhibitory activity when compared to cenersen (EL625). We anticipate that these particular oligos will exhibit enhanced inhibitory activity in cells with comparatively lower RNase H levels or when used in combination with mitoxantrone which is known to bind cenersen and other phosphorothioates but significantly less so to these types of compounds. They should also exhibit enhanced efficacy due to their reduced chemical reactivity particularly with platinum based compounds and NAPQi, the active metabolite of acetaminophen. Also, these variants should be more effective for the treatment of hypoxic tissues and should be more protective of normal tissues from inducers of p53 dependent programmed cell death, particularly in the cases where such cells are quiescent.

Surprisingly, compounds 17 and 18 stimulate p53 protein production. These are the first antisense oligos to our knowledge that stimulate p53 production. They will find therapeutic use that includes the treatment of cancers with wild type p53 by preferentially increasing the occurrence of p53-dependent programmed cell death in these cancers.

In certain instances it may be desirable to use more than one oligonucleotide directed to p53 for the treatment of medical conditions such as those listed in Table 1. Indeed, oligos could be chosen based on whether they act via a steric hindrance mechanism or trigger RNAse H activity and combined for treatment as previously discussed. Such treatments may be contemporaneous or separated for a period of time depending on the medical or other commercial need. For example, when treating cancer, the steric hindrance oligos are most efficient at protecting a wide range of differentiated cell types from the toxic effects of chemotherapy or radiation. They are also most useful for sensitizing the various cell types in a cancer to these same treatments. Since such steric hindrance oligos are in effect competitive inhibitors they must be present in cells at relatively high concentrations. In contrast, RNase H dependent oligos are much more restricted in terms of the cell types in which they are active. These cell types include stem cells and some, but not all, more mature cell types. RNase H dependent oligos also have the advantage that they work at lower concentrations in cells because the mechanism is catalytic. Thus, by way of example, for cancer treatment, steric hindrance oligos to p53 would be most efficient at cancer debulking (as a possible substitute for surgical debulking) and broad normal cell protection generally while RNase H dependent oligos to p53 would be most efficient at attacking cancer stem cells and protecting normal stem cells.

The RNase H dependent oligos provided in FIG. 1 could be combined with a steric hindrance oligo shown in Table 2. The steric hindrance oligos shown in Table 2 are preferably morpholinos and they may be covalently linked to a CPP or peptoid or be modified to have a charged backbone as described elsewhere herein. In the case of in vitro delivery the naked morpholios will not penetrate cells and the use of a peptoid or a CPP is the preferred method of achieving cellular uptake. In vivo morpholinos are taken up by cells with out the use of a carrier although the efficiency of uptake can be enhanced by a CPP or peptoid. The preferred CPPs for the present invention are (RX)₈B and R₆Pen the former of which was used in in vitro testing.

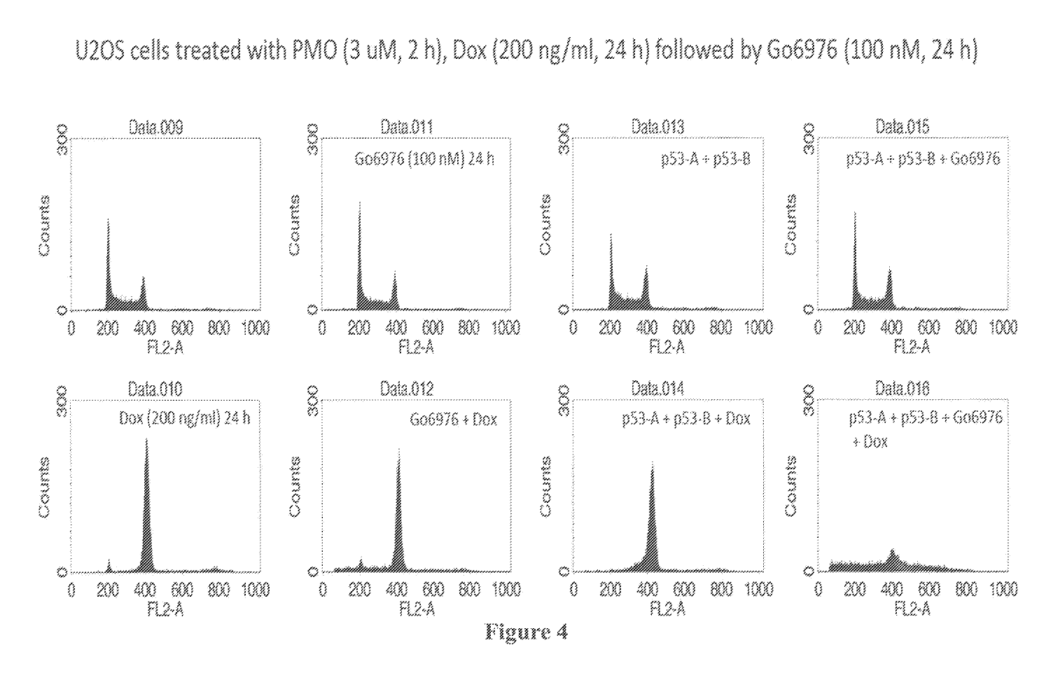

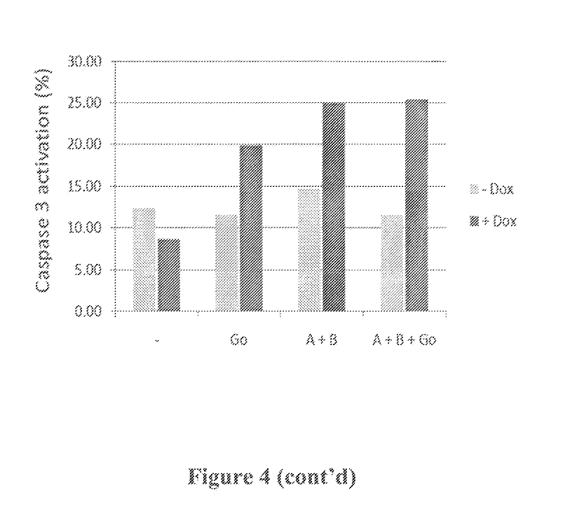

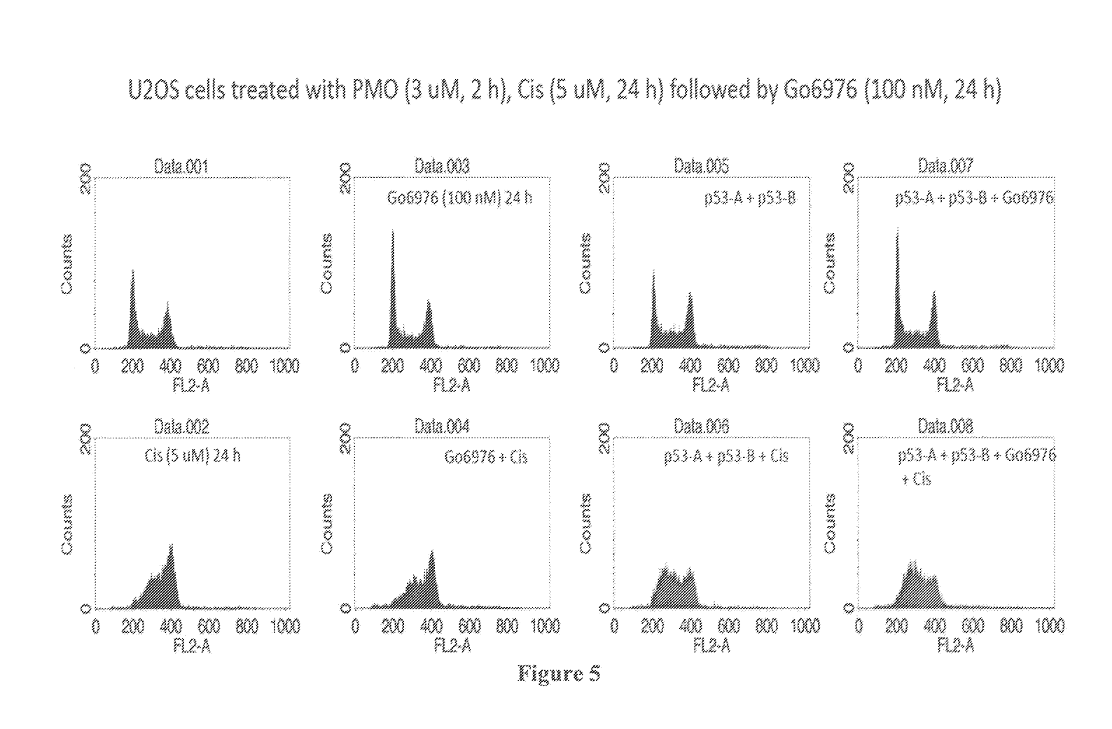

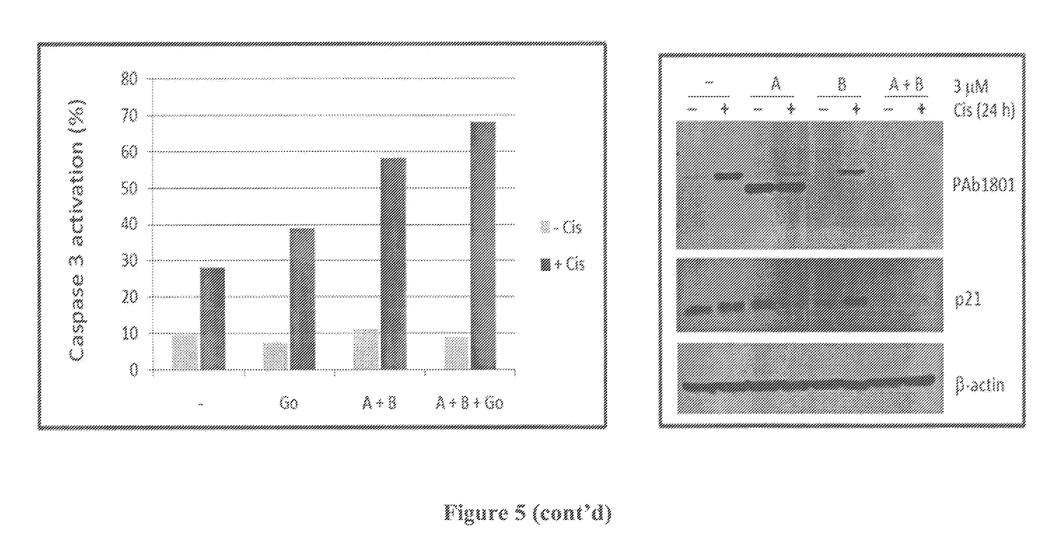

In further studies, additional agents were tested in conjunction with p53 morpholinos. FIG. 3 shows the results obtained when HL460 cells were treated with PMOs (p53-A and p53-B) +/−Dox+/−Go6976. We also tested these agents on U2OS cells (osteosarcoma). See FIG. 4. Again a dramatic effect on cell cycle that is dependent on PMOs and Go is observed. PMOs give a 3-fold enhancement of drug sensitivity in the caspase 3 assay. We also assessed U2OS cells treated with PMOs (p53-A and p53-B) +/−cisplatin+/−Go6976. These data reveal that PMOs A and B enhance sensitivity to cisplatin about 3-fold. Clearly Go6976 potentiates this enhancement. PMO A has a sequence that corresponds to Seq Id 136 and PMO B has a sequence that corresponds to Seq Id 140 shown in Table 2.

### Example 2

### Cell Based Method for Individualizing Treatment Protocols

Pharmacogenetic methods are increasingly being used to assist in matching cancer patients to particular drugs and/or treatment regiments particularly for the newer molecularly targeted therapies (Sawyers C L (2008). Nature 452: 548-552). Similar approaches will be useful for the optimization of treatment combinations and regimens based on the use of p53 suppressors. Important tests will include but not be limited to determining the following: (1) p53 status of the cancer to be treated with respect to somatic mutations, polymorphisms and post-translational modifications; (2) gains-of-function associated with mutant p53 expressed by this subset of cancers; (3) which cells in a given cancer are stem cells or progenitor cells enriched for stem cells so that molecular expression studied can be performed on this key cell population; (4) the status of cell cycle checkpoints determined by measuring cell cycle status and/or by determining the expression status of molecules involved in cell cycle checkpoint control following treatment with a p53 suppressor and/or genome damaging agent and/or with an agent that modulates cell cycle checkpoints; (5) the status of RNase H expression including but not limited to levels, subcellular compartmentalization and sufficiency with respect to supporting the antisense effect of a RNase H dependent p53 suppressor; (6) intracellular reactive oxygen species levels (ROS) and status of related mitochondrial function including but not limited to determining the mutational status of mitochondrial genes and/or nuclear gene that encode mitochondrial proteins; (7) expression levels and functional status of molecules that mediate p53-dependent programmed cell death and/or p53-dependent cell cycle arrest and genome repair; and (8) levels and functional status of molecules involved in modulating cell proliferation. Methods for such pharmacogenetic analysis are well known in the art and include but are not limited to the following: (1) immunohistochemistry and immunocytochemistry; (2) in situ hybridization; (3) DNA copy number assessment (comparative genome hybridization to DNA microarrays); (4) mutation screening (DNA sequencing, mass-spectrometry-based genotyping, mutation-specific PCR); (5) gene-expression profiling (DNA microarrays, multiplex PCR); (6) micro-RNA-expression profiling (DNA microarrays, multiplex PCR); (7) proteomic profiling (mass spectrometry) including after immunoprecipitation with antibodies to protein(s) of interest; (8) metabolic profiling (mass spectrometry). The following texts are incorporated herein by reference: (1) Pharmacogenomics: Methods and Protocols 2005 1st edition F. Innocenti editor Humana Press Totowa, N.J.; (2) Pharmacogenetics 2005 2nd edition W. Kalow et al. editors Informa Healthcare New York, N.Y.; (3) Pharmacogenetics and Personalized Medicine 2008 1st edition N. Cohen editor Humana Press Totowa, N.J.; and (4) Pharmacogenomics in Drug Discovery and Development 2008 1st edition Q. Yan editor Humana Press Totowa, N.J.

### Example 3

### In Vivo Administration of Oligonucleotides, Including p53 Inhibitors of the Invention to Subjects in Need Thereof

As for many drugs, dose schedules for treating patients with oligos can be readily extrapolated from animal studies. The extracellular concentrations that must be generally achieved with highly active conventional RNase H dependent antisense oligos is in the 1-200 nanomolar (nM) range. Higher extracellular levels, up to 1.5 micromolar, may be more appropriate for some applications as this can result in an increase in the speed and the amount of the oligos driven into the tissues. Higher doses also are needed for steric hindrance based antisense oligos particularly for those that do not use a carrier such as a CPP. The necessary levels can readily be achieved in plasma.

For in vivo applications, the concentration of the oligos to be used is readily calculated based on the volume of physiologic balanced-salt solution or other medium in which the tissue to be treated is being bathed. With fresh tissue, 1-1000 nM represents the concentration extremes needed for oligos with moderate to excellent activity. Two hundred nanomolar (200 nM) is a generally serviceable level for most applications. With most cell lines a carrier will typically be needed for in vitro administration. Incubation of the tissue with the oligos at 5% rather than atmospheric (ambient) oxygen levels may improve the results significantly.

Pharmacologic/toxicologic studies of phosphorothioate oligos, for example, have shown that they are adequately stable under in vivo conditions, and that they are readily taken up by all the tissues in the body following systemic administration with a few exceptions such as the central nervous system (Iversen, Anticancer Drug Design 6:531, 1991; Iversen, Antisense Res. Develop. 4:43, 1994; Crooke, Ann. Rev. Pharm. Toxicol. 32: 329, 1992; Cornish et al., Pharmacol. Comm. 3: 239, 1993; Agrawal et al., Proc. Natl. Acad. Sci. USA 88: 7595, 1991; Cossum et al., J. Pharm. Exp. Therapeutics 269: 89, 1994). These compounds readily gain access to the tissue in the central nervous system in large amounts following injection into the cerebral spinal fluid (Osen-Sand et al., Nature 364: 445, 1993; Suzuki et al., Amer J. Physiol. 266: R1418, 1994; Draguno et al., Neuroreport 5: 305, 1993; Sommer et al., Neuroreport 5: 277, 1993; Heilig et al., Eur. J. Pharm. 236: 339, 1993; Chiasson et al., Eur J. Pharm. 227: 451, 1992). Phosphorothioates per se have been found to be relatively non-toxic, and the class specific adverse effects that are seen occur at higher doses and at faster infusion rates than is needed to obtain a therapeutic effect with a well chosen sequence. In addition to providing for nuclease resistance, one potential advantage of phosphorothioate and boranophosphate linkages over the phosphodiester linkage is the promotion of binding to plasma proteins and albumin in particular with the resulting effect of an increased plasma half-life. By retaining the oligo for a longer period of time in plasma the oligo has more time to enter tissues as opposed to being excreted by the kidney. Oligos with primarily or exclusively phosphodiester linkages have a plasma half-life of only a few minutes. Thus, they are of little use for in vivo applications when used without a carrier. In the case of oligos with a preponderance or exclusively phosphodiester linkages, plasma protein binding can be improved by covalently attaching the oligo a molecule that binds a plasma protein such as serum albumin. Such molecules include, but are not limited to, an arylpropionic acid, for example, ibuprofen, suprofen, ketoprofen, pranoprofen, tiaprofenic acid, naproxen, flurpibrofen and carprofen. See U.S. Pat. No. 6,656,730. As for other moieties that might be linked to the oligos suitable for use with the present invention the preferred site is the 3′-end of the oligo. Intravenous administrations of oligos can be continuous for days or be administered over a period of minutes depending on the particular oligos and the medical indication. Phosphorothioate-containing oligos have been tested containing 18 nucleotides (e.g., oblimersen) to 20 nucleotides (e.g., cenersen, alicaforsen, aprinocarsen, ISIS 14803, ISIS 5132 and ISIS 2503) in length. When so administered such oligos show an alpha and a beta phase of elimination from the plasma. The alpha phase oligo half-life is 30 to 60 minutes while the beta phase is longer than two weeks for oligos with both phosphorothioate linkages and 2′-0 substitutions in at least the terminal four nucleosides on each end of the oligo.

The most prominent toxicities associated with intravenous administration of phosphorothioates have been related to the chemical class and generally independent of the mRNA target sequence and, therefore, independent of hybridization. The observed and measured toxicities have been consistent from drug to drug pre-clinically and clinically, with non-human primates being most similar to humans for certain key toxicities.

The class-related toxicities that have been most compelling in choosing dose and schedule for pre-clinical and clinical evaluation occur as a function of binding to specific plasma proteins and include transient inhibition of the clotting cascade and activation of the complement cascade. Both of these toxicities are thought to be related to the polyanionic nature of the molecules.

The effect of phosphorothioates on the clotting cascade results in plasma concentration-related prolongation of the activated partial thromboplastin (aPPT) time. Maximum prolongation of the aPTT correlates closely with the maximum plasma concentration so doses and schedules that avoid high peak concentrations can be selected to avoid significant effects on the aPTT. Because the plasma half-life of these drugs is short (30 to 60 minutes), the effect on clotting is transient. Several of these drugs have been evaluated in the clinic with prolonged intravenous infusions lasting up to 3 weeks. Shorter IV infusions (e.g., 2 hours) have also been studied. For example, aprinocarsen (ISIS 3521) and ISIS 5132 were studied with both 2 hour and 3-week continuous infusion schedules. At a dose of 3 mg/kg/dose over 2 hours, transient prolongation of the aPTT was observed. When 3 mg/kg was given daily by continuous infusion for 21 days, there was no effect on aPTT. The effect of antisense molecules of this chemical class on the clotting cascade is consistent.

Similarly, the activation of complement is a consistent observation; however, the relationship between plasma concentration of oligonucleotides and complement activation is more complex than the effect on clotting. Also, while the effect on clotting is found in rats as well as monkeys, the effect on the complement cascade has only been observed in monkeys and humans.

When these drugs are given to cynomolgus monkeys by 2-hour infusion, increases in complement split products (i.e., C3a, C5a, and Bb) occur only when plasma concentrations exceed a threshold value of 40-50 μg/mL. In monkeys, there is a low incidence of cardiovascular collapse associated with increases in these proteins. For the most part, clinical investigations of phosphorothioates have been designed to avoid these high plasma concentrations.

Cenersen has been evaluated in Rhesus monkeys using a 7-day continuous infusion schedule with a maximum dose of 27 mg/kg/day. In this study, minor Bb increases were noted in the highest dose group of 27 mg/kg/day with mean steady state plasma concentrations of cenersen measured in the 14-19 μg/mL range. Continuous intravenous schedules have not been evaluated in non-human primates with other oligonucleotides. Continuous infusions have been studied in clinical trials. Cenersen has been evaluated in a Phase I study at doses up to 0.25 mg/kg/hour for up to 10 days in patients with AML/MDS. ISIS 3521 was evaluated at doses up to 0.125 mg/kg/hour for 3 weeks and ISIS 5132 was evaluated at doses up to 0.21 mg/kg/hour for 3 weeks. In cancer patients treated with intermittent short infusions of ISIS 3521 and ISIS 5132 (2 hour infusions, given three times per week.) complement activation was not observed with doses up to 6 mg/kg (3 mg/kg/hour×2 hours) where mean peak plasma concentrations up to 30 μg/mL were recorded.

When ISIS 3521 was given as a weekly 24 hour infusion at doses as high as 24 mg/kg (1 mg/kg/hour×24 hours), the steady state plasma concentrations reached approximately 12 μg/mL at the high dose. On this schedule, however, there were substantial increases in C3a and Bb even though these plasma levels were much lower than those seen with the shorter infusions. Thus, activation of complement may be associated with both dose and schedule where plasma concentrations that are well tolerated over shorter periods of time (e.g. 2 hours), are associated with toxicity when the plasma concentrations are maintained for longer. This likely provides the explanation for the findings with cenersen in rhesus monkeys where complement activation was observed at concentrations of 14-19 μg/mL.

When ISIS 3521 was given at 1.0 and 1.25 mg/kg/hour×2 hours, the mean peak plasma concentrations were 11.1±0.98 and 6.82±1.33 ug/mL, respectively. There was no complement activation at these or other higher doses and no other safety issues. It is expected that the maximum peak plasma concentrations for similarly sized phosphorothioate given at 1.2 mg/kg/hour×1 hour would be similar to that observed with ISIS 3521.

Thus, infusion rates for phosphorothioates of 3.6 mg/kg/h or less are expected to be trouble free. With somewhat higher infusion rates the effects of complement activation can be expected. Decisions made about the sequential shortening of the infusion below one hour using a constant total dose of approximately 22 mg/kg should be readily achieved based on review of the safety information, including evaluation of complement split products.

These considerations set a range of dose and scheduling parameters particularly for in vivo use of the oligos of the present invention in situations where a carrier is not used.

### Example 4

### p53 Inhibitory Oligos of the Present Invention and Methods of Use Thereof for the Treatment of Diamond Blackfan Anemia

Diamond-Blackfan anemia (DBA) is characterized by anemia with decreased erythroid progenitors in the bone marrow. This usually develops during the neonatal period. DBA patients are thought to have a risk of developing leukemia and other malignancies although the data is not conclusive.

Individuals with DBA fail to make adequate red blood cells and in about 50% of cases carry mutations in one allele of any of several genes encoding ribosomal proteins, which are essential components of the protein synthesis machinery. RPS19 is the most frequently mutated RP in DBA. RPS19 deficiency resulting from the inactivating mutation results in a imbalance in the ribosomal proteins available for ribosome formation. Danilova et al. (Blood (2008) 112: 5228-37) report that rps19 deficiency in zebrafish results in hematopoietic and developmental abnormalities resembling DBA. These investigators have shown that suppression of p53 and deltaNp63 alleviates the rps19-deficient phenotype including anemia. Inactivating mutations in one allele of other ribosomal proteins, such as S8, S11, and S18 found in DBA patients, also appear to lead to up-regulation of p53 pathway, suggesting it is a common response to ribosomal protein deficiency.

In DBA cases where inactivating mutations in ribosomal proteins are not found other translations related abnormalities are likely the underlying cause of the disorder. Substantial clinical differences have not been seen between patients with or without demonstrated ribosomal mutations.

The chimeric oligonucleotides described herein down modulate expression of p53. Such oligos can be used to advantage to treat and ameliorate the symptoms of DBA and other disorders where ribosomal or other translations related defects lead to an activation of p53 expression. The sequence of an oligo effective to inhibit expression of p53 is provided in Example I. For the treatment of DBA, it is preferable to administer the oligo(s) of the invention systemically.

Several other disorders associated with abnormalities related to translation including other ribosomopathies also are well suited to being treated using the p53 inhibitors of the invention. Those currently designated as ribosomopathies characteristically exhibit refractory anemia and include Del (5q) MDS, Schwachman-Diamond syndrome, dyskeratosis congenita, cartilage hair hypoplasia, and Treacher Collins syndrome. In Del (5q) MDS the deletion eliminates one of the RPS14 alleles resulting in an imbalance in the ribosomal protein components in the same way that inactivating mutations produce this effect in DBA.

Those disorders involving translation related abnormalities including those with ribosomal abnormalities that have not been classified to date as ribosomopathies include refractory cytopenia, with unilineage dysplasia (e.g., refractory anemia, refractory neutropenia, and refractory thrombocytopenia), refractory anemia with ring sideroblasts, refractory cytopenia with multilineage dysplasia, refractory anemia with multilineage dysplasia, refractory anemia with excess blasts, type I, refractory anemia with excess blasts type II, refractory cytopenia of childhood and inherited bone marrow failure syndromes.

As with DBA above, systemic administration of an effective amount of a p53 inhibitor of the present invention alone or in combination with other drugs as previously described, should suppress or ameliorate the symptoms associated with these disorders. Such inhibitors may also be useful to prevent or impede progression to malignancy can occur in MDS for example.

### Example 5

### Use of p53 Inhibitory Oligos of the Present Invention in Cardiovascular Applications for the Treatment of Cardiovascular Disease

### A. Treatment of Cardiac Hypertrophy, MI, and Heart Failure.

Cardiovascular disease in the United States is associated with increasing morbidity and mortality and thus new therapeutic agents for the treatment of this disorder are highly desirable. Such diseases include atherosclerosis, atherosclerotic plaque rupture, aneurisms (and ruptures thereof), coronary artery disease, cardiac hypertrophy, restenosis, vascular calcification, vascular proliferative disease, myocardial infarction and related pathologies which include, apoptosis of cardiac muscle, heart wall rupture, and ischemia reperfusion injury.

While several different therapeutic approaches are currently available to manage cardiovascular disease, e.g., heart failure, the incidence, prevalence, and economic costs of the disease are steadily increasing. The overall prevalence of congestive heart failure (CHF) is 1 to 2% in middle-aged and older adults, reaches 2 to 3% in patients older than age 65 years, and is 5 to 10% in patients beyond the age of 75 years (Yamani et al. (1993) Mayo Clin. Proc. 68:1214-1218).

Survival of patients suffering from heart failure depends on the duration and severity of the disease, on gender, as well as on previously utilized therapeutic strategies. In the Framingham study, the overall 5-year survival rates were 25% in men and 38% in women (Ho et al., (1993) Circulation 88:107-115). In clinical trials with selected patients under state-of-the-art medical therapy, 1 year mortality ranged between 35% in patients with severe congestive heart failure (NYHA IV) in the Consensus trial (The Consensus Trial Study Group (1987) N Engl. J. Med. 316:1429-1435) to 9 and 12% in patients with moderate CHF (NYHA in the second Vasodilator Heart Failure Trial (Cohn et al. (1991) N. Engl. J. Med 325:303-310) and the Studies of Left Ventricular Dysfunction (SOLVD) trial. Mechanisms of death included sudden death in about 40%, and other factors in 20% of the patients.

The oligos of the invention can be employed to diminish or alleviate the pathological symptoms associated with cardiac cell death due to apoptosis of heart cells. Initially the p53 oligo of interest will be incubated with a cardiac cell and the ability of the oligo to modulate targeted gene function (e.g., reduction in production of target gene product, apoptosis, improved cardiac cell signaling, Ca++ transport, or morphology etc.) will be assessed. For example, the H9C2 cardiac muscle cell line can be obtained from American Type Culture Collection (Manassas, Va., USA) at passage 14 and cultured in DMEM complete culture medium (DMEM/F12 supplemented with 10% fetal calf serum (FCS), 2 mM α-glutamine, 0.5 mg/l Fungizone and 50 mg/l gentamicin). This cell line is suitable for characterizing the inhibitory functions of the oligos of the invention and for characterization of modified versions thereof. HL-1 cells, described by Clayton et al. (1998) PNAS 95:2979-2984, can be repeatedly passaged and yet maintain a cardiac-specific phenotype. These cells can also be used to further characterize the effects of the oligos described herein.

It may be desirable to further test the oligos of the invention in animal models of heart failure. Hasenfuss (1998) (Cardiovascular Research 39:60-76) provides a variety of animal models that are suitable for use in this embodiment of the invention. Each of the animal models described is useful for testing a biochemical parameter modulated by the oligos provided herein. The skilled person can readily select the appropriate animal model and assess the effects of the oligos for its ability to ameliorate the symptoms associated with heart disease.

Heart failure is a serious condition that results from various cardiovascular diseases. p53 plays a significant role in the development of heart failure. Cardiac angiogenesis directly related to the maintenance of cardiac function as well as the development of cardiac hypertrophy induced by pressure-overload, and up-regulated p53 induced the transition from cardiac hypertrophy to heart failure through the suppression of hypoxia inducible factor-1 (HIF-1), which regulates angiogenesis in the hypertrophied heart. In addition, p53 is known to promote apoptosis, and apoptosis is thought to be involved in heart failure. Thus, p53 is a key molecule which triggers the development of heart failure via multiple mechanisms.

It appears that expression of the apoptosis regulator p53 is governed, in part, by a molecule that in mice is termed murine double minute 2 (MDM2), or in man, human double minute 2 (HDM2), an E3 enzyme that targets p53 for ubiquitination and proteasomal processing, and by the de-ubiquitinating enzyme, herpesvirus-associated ubiquitin-specific protease (HAUSP), which rescues p53 by removing ubiquitin chains from it. Birks et al. (Cardiovasc Res. 2008 Aug. 1; 79(3):472-80) examined whether elevated expression of p53 was associated with dysregulation of ubiquitin-proteasome system (UPS) components and activation of downstream effectors of apoptosis in human dilated cardiomyopathy (DCM). In these studies, left ventricular myocardial samples were obtained from patients with DCM (n=12) or from non-failing (donor) hearts (n=17). Western blotting and immunohistochemistry revealed that DCM tissues contained elevated levels of p53 and its regulators HDM2, MDM2 or the homologs thereof found in other species, and HAUSP (all P<0.01) compared with non-failing hearts. DCM tissues also contained elevated levels of polyubiquitinated proteins and possessed enhanced 20S-proteasome chymotrypsin-like activities (P<0.04) as measured in vitro using a fluorogenic substrate. DCM tissues contained activated caspases-9 and -3 (P<0.001) and reduced expression of the caspase substrate PARP-1 (P<0.05). Western blotting and immunohistochemistry revealed that DCM tissues contained elevated expression levels of caspase-3-activated DNAse (CAD; P<0.001), which is a key effector of DNA fragmentation in apoptosis and also contained elevated expression of a potent inhibitor of CAD (ICAD-S; P<0.01). These investigators concluded that expression of p53 in human DCM is associated with dysregulation of UPS components, which are known to regulate p53 stability. Elevated p53 expression and caspase activation in DCM was not associated with activation of both CAD and its inhibitor, ICAD-S. These findings are consistent with the concept that apoptosis may be interrupted and therefore potentially reversible in human HF.

In view of the foregoing, it is clear that the oligos directed to p53 should exhibit efficacy for the treatment of heart failure. Accordingly, in one embodiment of the invention, an oligo to p53 is administered for inhibiting cardiac cell apoptosis for the treatment of heart failure.

### Example 6

### p53 Inhibitory Oligos of the Present Invention for the Treatment of Vascular Disorders

Atherosclerosis is a condition in which vascular smooth muscle cells are pathologically reprogrammed. Fatty material collects in the walls of arteries and there is typically chronic inflammation. This leads to a situation where the vascular wall thickens, hardens, forms plaques, which may eventually block the arteries or promote the blockage of arteries by promoting clotting. The latter becomes much more prevalent when there is a plaque rupture.

If the coronary arteries become narrow due to the effects of the plaque formation, blood flow to the heart can slow down or stop, causing chest pain (stable angina), shortness of breath, heart attack, and other symptoms. Pieces of plaque can break apart and move through the bloodstream. This is a common cause of heart attack and stroke. If the clot moves into the heart, lungs, or brain, it can cause a stroke, heart attack, or pulmonary embolism.

Risk factors for atherosclerosis include: diabetes, high blood pressure, high cholesterol, high-fat diet, obesity, personal or family history of heart disease and smoking. The following conditions have also been linked to atherosclerosis: cerebrovascular disease, kidney disease involving dialysis and peripheral vascular disease. Down modulation of p53 can have a beneficial therapeutic effect for the treatment of atherosclerosis and associated pathologies. WO/2007/030556 provides an animal model for assessing the effects of p53 directed oligos on the formation of atherosclerotic lesions.

Atherosclerotic plaque rupture is the main cause of coronary thrombosis and myocardial infarcts. Rekhter et al. have developed a rabbit model in which an atherosclerotic plaque can be ruptured at will after an inflatable balloon becomes embedded into the plaque. Furthermore, the pressure needed to inflate the plaque-covered balloon may be an index of overall plaque mechanical strength (Circulation Research. (1998) 83:705-713). The thoracic aorta of hypercholesterolemic rabbits underwent mechanical removal of endothelial cells, and then a specially designed balloon catheter was introduced into the lumen of the thoracic aorta. As early as 1 month after catheter placement, atherosclerotic plaque formed around the indwelling balloon. The plaques were reminiscent of human atherosclerotic lesions, in terms of cellular composition, patterns of lipid accumulation, and growth characteristics. Intraplaque balloons were inflated both ex vivo and in vivo, leading to plaque fissuring. The ex vivo strategy is designed to measure the mechanical strength of the surrounding plaque, while the in vivo scenario permits an analysis of the plaque rupture consequences, e.g., thrombosis. This model can be used to advantage for assessing local delivery of the p53 directed oligos described herein into the plaque in order to assess the effects of the same on plaque instability.

### Example 7

### Oligos Targeting Clusterin (SGP2, TRPM-2) for the Treatment of Disorders Characterized by Aberrant Apoptosis

Clusterin (also known as SGP2 or TRPM-2) is expressed in cells in multiple forms as reflected in differences in amino acid sequence and non-translated sequences that are involved in regulating expression of the corresponding protein. Andersen et al. (Mol Cell Proteomics 6: 1039, 2007) have described three variants of clusterin encoded proteins termed CLU34 (NCBI Reference Sequence NM_{—}001831), CLU35 (NCBI Reference Sequence NM_{—}203339) and CLU36 (sequence provided in supplemental information accompanying Andersen et al.). CLU 34 and CLU35 localize to the cytoplasm and are anti-apoptotic while CLU 36 is apoptotic and concentrates in the nucleus. The clusterin gene has a total of 9 exons. The mRNA variants described by Anderson et al. each possess different first exons. CLU 34 is the variant most commonly reported in the literature. It can be secreted by cells and has a variety of extracellular functions that include interactions with growth factor pathways, such interactions being associated with inhibition of apoptosis. Leskov et al., (J Biol Chem 278: 21055, 2003) have described yet another apoptotic form in addition to CLU36 that is derived from CLU34 by an alternative splicing mechanism that results in the deletion of exon 2. The primary translational start site for CLU34 is in its first exon while the primary start site for CLU35 is in exon 2. CLU36 has a primary start site in its first exon. Alternately spliced CLU34 has its primary translational start site in exon 3.

All three clusterin mRNA forms described by Andersen et al. are subject to differential regulation of their expression by various cellular processes that can be altered in diseased cells. For example, patterns of expression are typically altered in cancer cells such that expression levels of the anti-apoptotic variants are increased relative to the apoptotic variants. In prostate cancer, for example, CLU34 is repressed by androgens while CLU35 is up-regulated (Cochrane et al., J Biol Chem 282: 2278, 2007). Further, CLU35 is up-regulated in prostate cancer as it progresses to androgen independence.

Two homologs (CLI and SP-40,40) are also produced by the clusterin gene. These are distinguished by substantial divergences in the 5′ untranslated sequence particularly those in the general boundary region between intron I and exon II. This region includes hotspot 9 of the TRPM-2 gene set forth hereinbelow which can be targeted to differentially affect the expression of these homologs. Both of these homologs bind to complement components and inhibit complement mediated cellular lysis and are of importance in biological processes such as reproduction.

A conventional antisense oligo directed to clusterin with the sequence

SEQ ID NO: 3
(5′-CAGCAGCAGAGTC TTCATCAT-3′-)

is in development as a possible therapeutic agent (Schmitz, Current Opinion Mol Ther 8: 547, 2006; US 2004/0053874; 2008/0014198; 6,383,808; 6,900,187; 7,285,541; 7,368,436; WO 02/22635; 2006/056054). The terminal four nucleosides on each end of this oligo (indicated by underlining) have 2′-O-methyoxyethyl modifications to their sugar moieties. The linkages between all 21 nucleotides are phosphorothioate and the central 13 nucleosides all have deoxyribose as the sugar. It has been shown to modestly sensitize some cancer cells, including prostate cancer cells, to radiation and chemotherapeutic agents (Schmitz, Current Opinion Mol Ther 8: 547, 2006; Zellweger et al. (J Pharm Exp Ther 298: 934, 2001 and Clin Cancer Res 8: 3276, 2002). This oligo is directed to the primary translational start site for CLU35 in exon 2, but because it has an RNase H dependent mechanism of action rather than a steric hindrance mechanism of action, it indiscriminately also down-regulates CLU34 and CLU36 because they express the same exon 2. Thus, this oligo inhibits both anti-apoptotic and apoptotic forms of clusterin. Chen et al., (Cancer Res 64: 7412, 2004) have shown that this oligo can inhibit the induction of apoptosis in some cancer cells, including those deficient in p21 (WAF-1) expression, which is highly undesirable in a potential anti-cancer agent. This feature, along with its relatively poor suppressive activity on clusterin expression is associated with a relatively low level of therapeutic efficacy.

As for p53 targeting oligos, it may be desirable to use more than one oligo directed to clusterin (also referred to as SGP2 or TRPM-2) for the treatment of medical conditions such as those listed in Table 3 below.

Indeed, oligos could be chosen based on whether they act via a steric hindrance mechanism or trigger RNAse H activity and combined for treatment. Such treatments may be contemporaneous or separated for a period of time depending on the medical or other commercial need. The cancer example applied to the p53 oligos applies here as well, that is, when treating cancer the steric hindrance oligos are most efficient at sensitizing the various cell types in a cancer to cytotoxic treatments such as chemotherapy and radiation. Since such steric hindrance oligos are in effect competitive inhibitors they must be present in cells at relatively high concentrations. In contrast, RNase H dependent oligos are much more restricted in terms of the cell types were they are active. These cell types include stem cells and some but not all more mature cell types. RNase H dependent oligos also have the advantage that they work at lower concentrations in cells because the mechanism is catalytic. Thus, continuing the example, in cancer steric hindrance oligos to clusterin would be most efficient at cancer debulking (as a possible substitute for surgical debulking) while RNase H dependent oligos to clusterin would be most efficient at attacking cancer stem cells.

The RNase H dependent oligos provided in Tables 4A, 4B and 4C could be combined with a steric hindrance oligo shown in Tables 5-9 and 4D with those being directed to the primary translational start site being most preferred. Prototype oligos having an A designation in Tables 4A, 4B and 4C act to inhibit target gene expression via a steric hindrance mechanism. Prototype oligos having an OL designation in these tables act via triggering RNAse H action as do the oligos listed in Table 4D. Notably, steric hindrance oligos such as those set forth below in Tables 5-9 directed to the primary translational start site may be combined with those directed to the secondary translational start site. Further, these combinations of steric hindrance oligos may be used in combination with the RNase H dependent oligos provided in Table 4 A, B, C and D. The steric hindrance oligos shown in Tables 5-9 are preferably morpholinos and they may be covalently linked to a CPP or peptoid or be modified to have a charged backbone as described elsewhere herein. The most preferred CPPs are (RX)₈B and R₆Pen. One of these can be conjugated to either the 5′ or the 3′ end of the oligo.

Tables 4A, 4B, 4C and 4D provide prototype conventional antisense oligo sequences and their size variants that when combined with the preferred or most preferred backbones produce surprisingly better oligos with RNase H activity in terms of suppressing clusterin expression and in producing therapeutic effects such as sensitizing cancer cells to conventional cancer treatments or protecting nerve cells from the induction of apoptosis when compared to those clusterin targeting oligos provided in the prior art such as the one just described. Specifically, chimeric (DNA/RNA analog) 2′-fluoro and/or 2′-O-methyl or FANA or LNA containing oligos being preferred where any of these has at least 8 deoxyribose containing nucleosides in a row connected by phosphorothioate or boranophosphate linkages with phosphorothioate being preferred. In the case of FANA oligos only 3 but preferably 4 or 5 deoxyribose containing nucleosides in a row are required for RNase H activity. Most preferred are compounds in which the 5′ and 3′ 2-3 terminal nucleosides are LNA the center is 4 or 5 deoxyribose nucleosides and the rest are FANA.

As mentioned above, certain clusterin variants encode anti-apoptotic proteins while other variants possess apoptotic activities. When one or the other of these activities is not selectively blocked then the activity of the oligo will depend on which activity is dominant in any given situation. Selectively blocking the anti-apoptotic activity would be appropriate for treating a disorder such as cancer while selectively blocking apoptotic activity would be appropriate for the treatment of Alzheimer's Disease, for example. Table 3 lists several medical indications where oligos directed to clusterin should exhibit efficacy. These indications include both those characterized by pathologic induction of apoptosis as well as those where there is a pathologic resistance to the induction of apoptosis.

Clusterin transcripts encoding anti-apoptotic proteins can be selectively targeted by oligos using) conventional antisense oligos that support RNase H activity where the oligo binds to a segment of exon 1 of clusterin variant CLU34 (Hot Spot 4, SEQ ID NO: 13, in Table 4A) or to a segment of exon 1 of clusterin variant CLU35 (Hot Spot 2, SEQ ID NO: 24, in Table 4B); or (2) the use of conventional antisense oligos with selective steric hindrance activity against primary or both primary and secondary translational start sites for clusterin variant CLU 34 (Table 5) or with selective steric hindrance activity against primary or both primary and secondary or alternative secondary translational start sites for clusterin variant CLU35 (Table 6). Secondary translational start sites are used by cells when the primary translational start site is blocked such as by an antisense oligo with a steric hindrance mechanism.

In addition, an oligo directed to exon 1 of clusterin variant CLU34 may be used in combination with an oligo directed to exon 1 of clusterin variant CLU35 to simultaneously eliminate expression of both of these anti-apoptotic variants where the oligos involved are (a) conventional antisense oligos that support RNase H activity, (b) expression vectors or (c) siRNA or dicer substrates. For cancer treatment application such oligos will typically be used in combination with other agents that promote apoptosis such as chemotherapy, radiation and modulators of hormone activity in the case of hormonally dependent cancers.

Clusterin transcripts encoding apoptotic protein clusterin variant CLU36 can be selectively targeted by oligos using one of the following design considerations: (1) the use of conventional antisense oligos that support RNase H activity, expression vectors or guide strands that bind to exon 1 of clusterin variant CLU 36 (Table 4C, Hot Spot 3, SEQ ID NO: 39); or (2) the use of conventional antisense oligos with selective steric hindrance activity against the primary and its secondary translational start site (Table 8) or the alternative primary and its secondary translational start site (Table 8).

Clusterin transcripts encoding apoptotic protein that is produced by the removal of exon 2 by alternative splicing of CLU34 can be selectively targeted by oligos by the use of conventional antisense oligos with selective steric hindrance activity against primary or both primary and secondary translational start sites in exon 3 (Table 9).

Tables 4A, 4B, 4C, and 4D provide for each clusterin hot spot (presented as an antisense sequence) at least one prototype conventional antisense oligo sequence along with a listing of size variant oligo sequences that are suitable for use in oligos in accordance with the present invention.

The use of particular primary or secondary start sites, where they occur on a tissue specific basis, can be readily determined using monoclonal antibodies directed to protein sequences that would appear upstream or downstream of particular translational start sites to determine whether or not the start site is being utilized. If it is used the upstream sequence will not be seen in a Western or similar blot or other appropriate assay method and the downstream sequence will be seen. If it is not used both protein sequences will be recognized.

Oligos which block the anti-apoptotic effects of clusterin variants are particularly desirable for the treatment of prostate cancer. Such oligos can be administered systemically or directly injected into the tumor. They can be used in combination with chemotherapy, biotherapy or radiation considered appropriate for the cancer. The treatment regimens set forth above may also comprise administration of chemotherapeutic agents such as abarelix, abiraterone acetate and Degarelix.

TABLE 2
Prototype Primary Start Site Steric Hindrance Antisense Oligos and Prototype Secondary Start Site
Steric Hindrance Antisense Oligos: Single and Duel Antisense Oligo Combinations Targeting p53
for p53 Related Clinical Indications Shown in Table 1

| | Primary Translational | SEQ ID | Secondary Translational | SEQ ID |
|---|---|---|---|---|
| Content of Row | Start Site | NO: | Start Sites | NO: |
| Hotspot for Primary Transitional | GACGCTAGGA TCTGACTGCG | 135 | Not Applicable | — |
| Start Site Region | GCTCCTCCAT GGCAGTGACC | | | |
| | CGGAAGGC | | | |
| Prototype Primary Transitional | NU(4)p53: CCATGGCAGT | 136 | Not Applicable | — |
| Start Site Antisense Oligo #1 | GACCCGGAAG GC | | | |
| Prototype Primary Transitional | NU(5)p53: GCGGCTCCTC | 137 | Not Applicable | — |
| Start Site Antisense Oligo #2 | CATGGCAGTG A | | | |
| Prototype Primary Transitional | NU(6)p53: TCTGACTGCG | 138 | | |
| Start Site Antisense Oligo #3 | GCTCCTCCAT GG | | | |
| Hotspot for Secondary Transitional | Not Applicable | — | TCAATATCGT CCGGGGACAG | 139 |
| Start Site Region | | | CATCAAATCA TCCATTGCTT G | |
| Combination #1 Prototype Primary | NU(4)p53: CCATGGCAGT | 136 | NU(7)p53: GCATCAAATC | 140 |
| and Secondary Transitional Start | GACCCGGAAG GC | | ATCCATTGCT TG | |
| Site Antisense Oligos | | | | |
| Combination #2 Prototype Primary | NU(4)p53: CCATGGCAGT | 136 | NU(8)p53: GGGACAGCAT | 141 |
| and Secondary Transitional Start | GACCCGGAAG GC | | CAAATCATCC AT | |
| Site Antisense Oligos | | | | |
| Combination #3 Prototype Primary | NU(5)p53: GCGGCTCCTC | 137 | NU(7)p53: GCATCAAATC | 140 |
| and Secondary Transitional Start | CATGGCAGTG A | | ATCCATTGCT TG | |
| Site Antisense Oligos | | | | |
| Combination #4 Prototype Primary | NU(5)p53: GCGGCTCCTC | 137 | NU(8)p53: GGGACAGCAT | 141 |
| and Secondary Transitional Start | CATGGCAGTG A | | CAAATCATCC AT | |
| Site Antisense Oligos | | | | |
| Combination #5 Prototype Primary | NU(6)p53: TCTGACTGCG | 138 | NU(7)p53: GCATCAAATC | 140 |
| and Secondary Transitional Start | GCTCCTCCAT GG | | ATCCATTGCT TG | |
| Site Antisense Oligos | | | | |
| Combination #6 Prototype Primary | NU(6)p53: TCTGACTGCG | 138 | NU(8)p53: GGGACAGCAT | 141 |
| and Secondary Transitional Start | GCTCCTCCAT GG | | CAAATCATCC AT | |
| Site Antisense Oligos | | | | |

TABLE 3

| | |
|---|---|
| Clusterin(SGP2; CLU; TRPM-2; | Cancers expressing this gene; Sensitizing cancers to chemotherapy, radiation or other genotoxic anti-cancer |
| Apolipoprotein J; APOJ; Complement | agents; Cervical cancer; Laryngeal squamous cell carcinoma; Osteosarcoma; Liver cancer; Colorectal |
| associated protein SP-40, 40; | cancer; Ovarian cancer; Bladder cancer; Breast cancer including sensitizing breast cancer to biologics in |
| Complement cytolysis inhibitor; | the case of breast cancers responsive to hormonal-pathway manipulation including sensitizing them to the |
| KUB1; CLI; Testosterone-repressed | use of estrogen antagonists; Prostate cancer including sensitizing this cancer to biologies in the case of |
| prostate message 2) | prostate cancers responsive to hormonal-pathway manipulation including the use of estrogen therapy, |
| | androgen deprivation therapy including gonadotropin-releasing hormone antagonists and luteinizing |
| | hormone-releasing hormone agonists (LHRH analogs) as well as cytochrome P450(17alpha)/C17-20 lyase |
| | inhibitors such as abiraterone; Preeclampsia; Early stage atherosclerosis; Fuch's endothelial dystrophy |

TABLE 4A
Gene: Clusterin (CLU34)
NCBI Reference Sequence: NM_001831
Reference: Andersen et al., Mol Cell Proteomics 6: 1039, 2007.
HOT-SPOT 1. Range of bases included: positions 9-40*
Antisense Strand Sequence:
SEQ ID NO: 4: GCAAACCTGC ATGACTCACG CCCAAAGAAT GC
Note: Size variants for this Hot Spot were selected as targets for conventional antisense oligos
with steric hindrance function against the translational start site in exon 1

| Nucleotide | |
|---|---|
| Starting | Size Variants |
| Position* | (Number of bases in the oligomer) |
| 9 | 22, 23 |
| 10 | 21, 22, 23, 24, 25, 26, 27, 28, 29, 30, 31 |
| 11 | 20, 21, 22, 23, 24, 25, 26, 27, 28, 29, 30 |
| 12 | 19, 20, 21, 22, 23, 24, 25, 26, 27, 28, 29 |
| 13 | 18, 19, 20, 21, 22, 23, 24, 25, 26, 27, 28 |
| 14 | 17, 18, 19, 20, 21, 22, 23, 24, 25, 26, 27 |
| 15 | 16, 17, 18, 19, 20, 21, 22, 23, 24, 25, 26 |
| 16 | 16, 17, 18, 19, 20, 21, 22, 23, 24, 25 |
| 17 | 16, 17, 18, 19, 20, 21, 22, 23, 24 |
| 18 | 16, 17, 18, 19, 20, 21, 22, 23 |
| 19 | 16, 17, 18, 19, 20, 21, 22 |

Prototype Oligonucleotides:

| Sequence | Trivial | Starting | |
|---|---|---|---|
| ID No. | Name | Position* | 5′-->3′ Sequence |
| 5 | A(1)CLU34 | 12 | TGCATGACTC ACGCCCAAAG AA |
| 6 | A(2)CLU34 | 19 | GCAAACCTGC ATGACTCACG CC |

Gene: Clusterin (CLU34)
NCBI Reference Sequence: NM_001831
Reference: Andersen et al., Mol Cell Proteomics 6: 1039, 2007.
HOT-SPOT 2. Range of bases included: positions 264-298*
Antisense Strand Sequence:
SEQ ID NO: 7: CCCTGATTGG ACATTTCCTG GAGCTCATTGT CTGA
Note: Size variants for this Hot Spot were selected as targets for conventional antisense oligos
with steric hindrance function against the primary translational start site for alternatively
spliced CLU34 in exon 3

| Nucleotide | |
|---|---|
| Starting | Size Variants |
| Position* | (Number of bases in the oligomer) |
| 264 | 24 |
| 265 | 23, 24, 25, 26, 27, 28, 29, 30, 31, 32 |
| 266 | 22, 23, 24, 25, 26, 27, 28, 29, 30, 31, 32 |
| 267 | 21, 22, 23, 24, 25, 26, 27, 28, 29, 30, 31, 32 |
| 268 | 20, 21, 22, 23, 24, 25, 26, 27, 28, 29, 30, 31 |
| 269 | 19, 20, 21, 22, 23, 24, 25, 26, 27, 28, 29, 30 |
| 270 | 18, 19, 20, 21, 22, 23, 24, 25, 26, 27, 28, 29 |
| 271 | 17, 18, 19, 20, 21, 22, 23, 24, 25, 26, 27, 28 |
| 272 | 16, 17, 18, 19, 20, 21, 22, 23, 24, 25, 26, 27 |
| 273 | 16, 17, 18, 19, 20, 21, 22, 23, 24, 25, 26 |
| 274 | 16, 17, 18, 19, 20, 21, 22, 23, 24, 25 |
| 275 | 16, 17, 18, 19, 20, 21, 22, 23, 24 |
| 276 | 16, 17, 18, 19, 20, 21, 22, 23 |
| 277 | 16, 17, 18, 19, 20, 21, 22 |
| 278 | 16, 17, 18, 19, 20, 21 |
| 279 | 16, 17, 18, 19, 20 |
| 280 | 16, 17, 18, 19 |
| 281 | 16, 17, 18 |

Prototype Oligonucleotides:

| Sequence | Trivial | Starting | |
|---|---|---|---|
| ID No. | Name | Position* | 5′-->3′ Sequence |
| 8 | A(3)CLU34 | 269 | GGACATTTCC TGGAGCTCAT TG |
| 9 | A(4)CLU34 | 277 | CCCTGATTGG ACATTTCCTG GA |

Gene: Clusterin (CLU34)
NCBI Reference Sequence: NM_001831
Reference: Andersen et al., Mol Cell Proteomics 6: 1039, 2007.
HOT-SPOT 3. Range of bases included: positions 475-515*
Antisense Strand Sequence:
SEQ ID NO: 10: CTCAGAGGGC CATCATGGTC TCATTGCACA CTCCTGGGAG
Note: Size variants for this Hot Spot were selected as targets for conventional antisense oligos
with steric hindrance function against the secondary translational start site for alternatively
spliced CLU34 in exon 4

| Nucleotide | |
|---|---|
| Starting | Size Variants |
| Position* | (Number of bases in the oligomer) |
| 475 | 32 |
| 476 | 31, 32 |
| 477 | 30, 31, 32 |
| 478 | 29, 30, 31, 32 |
| 479 | 28, 29, 30, 31, 32 |
| 480 | 27, 28, 29, 30, 31, 32 |
| 481 | 26, 27, 28, 29, 30, 31, 32 |
| 482 | 25, 26, 27, 28, 29, 30, 31, 32 |
| 483 | 24, 25, 26, 27, 28, 29, 30, 31, 32 |
| 484 | 23, 24, 25, 26, 27, 28, 29, 30, 31 |
| 485 | 22, 23, 24, 25, 26, 27, 28, 29, 30 |
| 486 | 21, 22, 23, 24, 25, 26, 27, 28, 29 |
| 487 | 20, 21, 22, 23, 24, 25, 26, 27, 28 |
| 488 | 19, 20, 21, 22, 23, 24, 25, 26, 27 |
| 489 | 18, 19, 20, 21, 22, 23, 24, 25, 26 |
| 490 | 17, 18, 19, 20, 21, 22, 23, 24, 25 |
| 491 | 16, 17, 18, 19, 20, 21, 22, 23, 24 |
| 492 | 16, 17, 18, 19, 20, 21, 22, 23 |
| 493 | 16, 17, 18, 19, 20, 21, 22 |
| 494 | 16, 17, 18, 19, 20, 21 |
| 495 | 16, 17, 18, 19, 20 |
| 496 | 16, 17, 18, 19 |
| 497 | 16, 17, 18 |
| 498 | 16, 17 |
| 499 | 16 |

Prototype Oligonucleotides:

| Sequence | Trivial | Starting | |
|---|---|---|---|
| ID No. | Name | Position* | 5′-->3′ Sequence |
| 11 | A(5)CLU34 | 489 | GGGCCATCAT GGTCTCATTG CA |
| 12 | A(6)CLU34 | 496 | CAGAGGGCCA TCATGGTCT |

Gene: Clusterin (CLU34)
NCBI Reference Sequence: NM_001831
Reference: Andersen et al., Mol Cell Proteomics 6: 1039, 2007.
HOT-SPOT 4. Range of bases included: positions 1-153*
Antisense Strand Sequence:
SEQ ID NO: 13: CCGGTCACGG ACCCTGTGCC CATGCTGCTG CTCGTCACGG ACCCTGTGCC
CATGCTGCTG CTCCTGGCGA CGCCGCGTTG TGGGCACTGG GAGGCGCCGT ATTTATAGCG
CTCCGTTCGC GCACACACCT TTGGGGCTGG CTGCAAACCT GCATGACTCA CGCCCAAAGA
ATGCCGCGGA AAG

| Nucleotide | |
|---|---|
| Starting | Size Variants |
| Position* | (Number of bases in the oligomer) |
| 1 | 16, 17, 18, 19, 20, 21, 22 |
| 2 | 16, 17, 18, 19, 20, 21 |
| 3 | 16, 17, 18, 19, 20 |
| 4 | 16, 17, 18, 19 |
| 5 | 16, 17, 18 |
| 6 | 16, 17 |
| 7 | 16, 17, 18, 19, 20, 21, 22, 23, 24, 25, 26 |
| 8 | 16, 17, 18, 19, 20, 21, 22, 23, 24, 25 |
| 9 | 16, 17, 18, 19, 20, 21, 22, 23, 24 |
| 10 | 16, 17, 18, 19, 20, 21, 22, 23 |
| 11 | 16, 17, 18, 19, 20, 21, 22, 23, 24, 25, 26, 27, 28, 29, 30, 31 |
| 12 | 16, 17, 18, 19, 20, 21, 22, 23, 24, 25, 26, 27, 28, 29, 30 |
| 13 | 16, 17, 18, 19, 20, 21, 22, 23, 24, 25, 26, 27, 28, 29 |
| 14 | 16, 17, 18, 19, 20, 21, 22, 23, 24, 25, 26, 27, 28 |
| 15 | 16, 17, 18, 19, 20, 21, 22, 23, 24, 25, 26, 27 |
| 16 | 16, 17, 18, 19, 20, 21, 22, 23, 24, 25, 26 |
| 17 | 16, 17, 18, 19, 20, 21, 22, 23, 24, 25 |
| 18 | 16, 17, 18, 19, 20, 21, 22, 23, 24 |
| 19 | 16, 17, 18, 19, 20, 21, 22, 23 |
| 20 | 16, 17, 18, 19, 20, 21, 22 |
| 21 | 16, 17, 18, 19, 20, 21 |
| 22 | 16, 17, 18, 19, 20 |
| 23 | 16, 17, 18, 19 |
| 24 | 16, 17, 18 |
| 25 | 16, 17 |
| 26 | 16 |
| 32 | 16, 17, 18, 19, 20, 21, 22 |
| 33 | 16, 17, 18, 19, 20, 21 |
| 34 | 16, 17, 18, 19, 20 |
| 35 | 16, 17, 18, 19 |
| 36 | 16, 17, 18 |
| 37 | 16, 17 |
| 38 | 16, 17, 18, 19, 20, 21, 22, 23, 24, 25, 26, 27, 28, 29, 30, 31, 32 |
| 39 | 16, 17, 18, 19, 20, 21, 22, 23, 24, 25, 26, 27, 28, 29, 30, 31, 32 |
| 40 | 16, 17, 18, 19, 20, 21, 22, 23, 24, 25, 26, 27, 28, 29, 30, 31, 32 |
| 41 | 16, 17, 18, 19, 20, 21, 22, 23, 24, 25, 26, 27, 28, 29, 30, 31, 32 |
| 42 | 16, 17, 18, 19, 20, 21, 22, 23, 24, 25, 26, 27, 28, 29, 30, 31, 32 |
| 43 | 16, 17, 18, 19, 20, 21, 22, 23, 24, 25, 26, 27, 28, 29, 30, 31, 32 |
| 44 | 16, 17, 18, 19, 20, 21, 22, 23, 24, 25, 26, 27, 28, 29, 30, 31 |
| 45 | 16, 17, 18, 19, 20, 21, 22, 23, 24, 25, 26, 27, 28, 29, 30 |
| 46 | 16, 17, 18, 19, 20, 21, 22, 23, 24, 25, 26, 27, 28, 29 |
| 47 | 16, 17, 18, 19, 20, 21, 22, 23, 24, 25, 26, 27, 28 |
| 48 | 16, 17, 18, 19, 20, 21, 22, 23, 24, 25, 26, 27 |
| 49 | 16, 17, 18, 19, 20, 21, 22, 23, 24, 25, 26 |
| 50 | 16, 17, 18, 19, 20, 21, 22, 23, 24, 25 |
| 51 | 16, 17, 18, 19, 20, 21, 22, 23, 24 |
| 52 | 16, 17, 18, 19, 20, 21, 22, 23 |
| 53 | 16, 17, 18, 19, 20, 21, 22 |
| 54 | 16, 17, 18, 19, 20, 21 |
| 55 | 16, 17, 18, 19, 20 |
| 56 | 16, 17, 18, 19 |
| 57 | 16, 17, 18 |
| 58 | 16, 17 |
| 59 | 16 |
| 66 | 16, 17, 18, 19, 20, 21, 22, 23 |
| 67 | 16, 17, 18, 19, 20, 21, 22 |
| 68 | 16, 17, 18, 19, 20, 21 |
| 69 | 16, 17, 18, 19, 20 |
| 70 | 16, 17, 18, 19 |
| 71 | 16, 17, 18 |
| 72 | 16, 17 |
| 73 | 16 |
| 75 | 16, 17, 18, 19, 20, 21, 22, 23, 24, 25, 26 |
| 76 | 16, 17, 18, 19, 20, 21, 22, 23, 24, 25 |
| 77 | 16, 17, 18, 19, 20, 21, 22, 23, 24 |
| 78 | 16, 17, 18, 19, 20, 21, 22, 23 |
| 79 | 16, 17, 18, 19, 20, 21, 22 |
| 80 | 16, 17, 18, 19, 20, 21 |
| 81 | 16, 17, 18, 19, 20 |
| 82 | 16, 17, 18, 19 |
| 83 | 16, 17, 18 |
| 84 | 16, 17 |
| 85 | 16 |
| 87 | 16, 17, 18, 19, 20, 21, 22 |
| 88 | 16, 17, 18, 19, 20, 21 |
| 89 | 16, 17, 18, 19, 20, 21 |
| 90 | 16, 17, 18, 19, 20, 21, 22 |
| 91 | 16, 17, 18, 19, 20, 21 |
| 92 | 16, 17, 18, 19, 20 |
| 93 | 16, 17, 18, 19 |
| 94 | 16, 17, 18 |
| 95 | 16, 17 |
| 96 | 16 |
| 109 | 16, 17, 18, 19, 20, 21, 22, 23, 24, 25 |
| 110 | 16, 17, 18, 19, 20, 21, 22, 23, 24 |
| 111 | 16, 17, 18, 19, 20, 21, 22, 23 |
| 112 | 16, 17, 18, 19, 20, 21, 22 |
| 113 | 16, 17, 18, 19, 20, 21 |
| 114 | 16, 17, 18, 19, 20 |
| 115 | 16, 17, 18, 19 |
| 116 | 16, 17, 18 |
| 117 | 16, 17 |
| 118 | 16, 17, 18, 19, 20, 21, 22, 23, 24, 25, 26, 27, 28, 29, 30, 31, 32 |
| 119 | 16, 17, 18, 19, 20, 21, 22, 23, 24, 25, 26, 27, 28, 29, 30, 31, 32 |
| 120 | 16, 17, 18, 19, 20, 21, 22, 23, 24, 25, 26, 27, 28, 29, 30, 31, 32 |
| 121 | 16, 17, 18, 19, 20, 21, 22, 23, 24, 25, 26, 27, 28, 29, 30, 31, 32 |
| 122 | 16, 17, 18, 19, 20, 21, 22, 23, 24, 25, 26, 27, 28, 29, 30, 31 |
| 123 | 16, 17, 18, 19, 20, 21, 22, 23, 24, 25, 26, 27, 28, 29, 30 |
| 124 | 16, 17, 18, 19, 20, 21, 22, 23, 24, 25, 26, 27, 28, 29 |
| 125 | 16, 17, 18, 19, 20, 21, 22, 23, 24, 25, 26, 27, 28 |
| 126 | 16, 17, 18, 19, 20, 21, 22, 23, 24, 25, 26, 27 |
| 127 | 16, 17, 18, 19, 20, 21, 22, 23, 24, 25, 26 |
| 128 | 16, 17, 18, 19, 20, 21, 22, 23, 24, 25 |
| 129 | 16, 17, 18, 19, 20, 21, 22, 23, 24 |
| 130 | 16, 17, 18, 19, 20, 21, 22, 23 |
| 131 | 16, 17, 18, 19, 20, 21, 22 |
| 132 | 16, 17, 18, 19, 20, 21 |
| 133 | 16, 17, 18, 19, 20 |
| 134 | 16, 17, 18, 19 |
| 135 | 16, 17, 18 |
| 136 | 16, 17 |
| 137 | 16 |

Prototype Oligonucleotides:

| Sequence | Trivial | Starting | |
|---|---|---|---|
| ID No. | Name | Position* | 5′-->3′ Sequence |
| 14 | OL(1)CLU34 | 3 | GCCCAAAGAA TGCCGCGGAA |
| 15 | OL(2)CLU34 | 32 | GGGCTGGCTG CAAACCTGC |
| 16 | OL(3)CLU34 | 55 | GCTCCGTTCG CGCACACACC |
| 17 | OL(4)CLU34 | 92 | CCGCGTTGTG GGCACTGGGA |
| 18 | OL(5)CLU34 | 109 | TGCTGCTCCT GGCGACGCCG |
| 19 | OL(6)CLU34 | 123 | CCCTGTGCCC ATGCTGCTGC |
| 20 | OL(7)CLU34 | 133 | CGGTCACGGA CCCTGTGCCC |

*Because this is antisense, the position numbers must be read right to left; the starting and ending positions are defined according to the NCBI Reference Sequence with the first base of the sense strand being base number one.

TABLE 4B
Gene: Clusterin (CLU35)
NCBI Reference Sequence: NM_203339 supplemented with 16 bases added to 5′-end as provided in
supplemental material provided with Anderson et al. reference
Reference: Andersen et al., Mol Cell Proteomics 6: 1039, 2007.
HOT-SPOT 1. Range of bases included: positions 292-323*
Antisense Strand Sequence:
SEQ ID NO: 21: CAGCAGAGTC TTCATCATGC CTCCAATTCT TG

| Nucleotide Starting | Size Variants |
|---|---|
| Position* | (Number of bases in the oligomer) |
| 292 | 19, 20, 21, 22, 23, 24, 25, 26, 27, 28, 29 |
| 293 | 18, 19, 20, 21, 22, 23, 24, 25, 26, 27, 28 |
| 294 | 17, 18, 19, 20, 21, 22, 23, 24, 25, 26, 27 |
| 295 | 16, 17, 18, 19, 20, 21, 22, 23, 24, 25, 26 |
| 296 | 16, 17, 18, 19, 20, 21, 22, 23, 24, 25 |
| 297 | 16, 17, 18, 19, 20, 21, 22, 23, 24 |
| 298 | 16, 17, 18, 19, 20, 21, 22, 23 |
| 299 | 16, 17, 18, 19, 20, 21, 22 |
| 300 | 16, 17, 18, 19, 20, 21 |
| 301 | 16, 17, 18, 19, 20 |
| 302 | 16, 17, 18, 19 |
| 303 | 16, 17, 18 |
| 304 | 17 |
| 305 | 17, 18, 19, 20, 21, 22, 23, 24, 25, 26, 27, 28, 29, 30, 31, 32 |
| 306 | 17, 18, 19, 20, 21, 22, 23, 24, 25, 26, 27, 28, 29, 30, 31, 32 |
| 307 | 17, 18, 19, 20, 21, 22, 23, 24, 25, 26, 27, 28, 29, 30, 31, 32 |

Prototype Oligonucleotides:

| Sequence | Trivial | Starting | |
|---|---|---|---|
| ID No. | Name | Position* | 5′-->3′ Sequence |
| 22 | A(1)CLU35 | 292 | TTCATCATGC CTCCAATTCT GG |
| 23 | A(2)CLU35 | 303 | CAGCAGCAGA GTCTTCATCA TG |

The following oligos have an RNase H mechanism of action.
Gene: Clusterin (CLU35)
NCBI Reference Sequence: NM_203339 supplemented with 16 bases added to 5′-end as provided in
supplemental material provided with Anderson et al. reference
Reference: Andersen et al., Mol Cell Proteomics 6: 1039, 2007.
HOT-SPOT 2. Range of bases included: positions 1-269*
Antisense Strand Sequence:
SEQ ID NO: 24:
CTTGACTTTC AAGACGCGGA GGCAACAGCA GCCAGCCCTT CACACCGAAT CCATCTGCAT
CCTAGTGGGA GACTTGGGCC GGGCTGCCTG TGCATTCAGG GGTGGAAGTA GTGGAAGCCA
GGAAGGACAA TTCCTTCGGA GAGTAGAGAG GGTTCGCAGT GGCCCGAGGA GCAGGCTTCC
CAGAGAAAGT CCCTTTGGAA GGATGAGTCC TCTTGCTCTG CCAGGCCCTC CACACTGCCC
ATCCCCTCTA AGCCGACACG AGGCTGCCC

| Nucleotide Starting | Size Variants |
|---|---|
| Position* | (Number of bases in the oligomer) |
| 1 | 16, 17 |
| 2 | 16 |
| 7 | 16, 17, 18 |
| 8 | 16, 17 |
| 9 | 16, 17, 18, 19, 20, 21, 22, 23, 24, 25, 26, 27, 28, 29, 30, 31, 32 |
| 10 | 16, 17, 18, 19, 20, 21, 22, 23, 24, 25, 26, 27, 28, 29, 30, 31, 32 |
| 11 | 16, 17, 18, 19, 20, 21, 22, 23, 24, 25, 26, 27, 28, 29, 30, 31, 32 |
| 12 | 16, 17, 18, 19, 20, 21, 22, 23, 24, 25, 26, 27, 28, 29, 30, 31, 32 |
| 13 | 16, 17, 18, 19, 20, 21, 22, 23, 24, 25, 26, 27, 28, 29, 30, 31, 32 |
| 14 | 16, 17, 18, 19, 20, 21, 22, 23, 24, 25, 26, 27, 28, 29, 30, 31, 32 |
| 15 | 16, 17, 18, 19, 20, 21, 22, 23, 24, 25, 26, 27, 28, 29, 30, 31 |
| 16 | 16, 17, 18, 19, 20, 21, 22, 23, 24, 25, 26, 27, 28, 29, 30 |
| 17 | 16, 17, 18, 19, 20, 21, 22, 23, 24, 25, 26, 27, 28, 29 |
| 18 | 16, 17, 18, 19, 20, 21, 22, 23, 24, 25, 26, 27, 28 |
| 19 | 16, 17, 18, 19, 20, 21, 22, 23, 24, 25, 26, 27 |
| 20 | 16, 17, 18, 19, 20, 21, 22, 23, 24, 25, 26 |
| 21 | 16, 17, 18, 19, 20, 21, 22, 23, 24, 25 |
| 22 | 16, 17, 18, 19, 20, 21, 22, 23, 24 |
| 23 | 16, 17, 18, 19, 20, 21, 22, 23 |
| 24 | 16, 17, 18, 19, 20, 21, 22 |
| 25 | 16, 17, 18, 19, 20, 21 |
| 26 | 16, 17, 18, 19, 20 |
| 27 | 16, 17, 18, 19 |
| 28 | 16, 17, 18, |
| 29 | 16, 17 |
| 30 | 16 |
| 32 | 16, 17, 18, 19, 20, 21, 22, 23, 24, 25, 26, 27, 28, 29 |
| 33 | 16, 17, 18, 19, 20, 21, 22, 23, 24, 25, 26, 27, 28 |
| 34 | 16, 17, 18, 19, 20, 21, 22, 23, 24, 25, 26, 27 |
| 35 | 16, 17, 18, 19, 20, 21, 22, 23, 24, 25, 26 |
| 36 | 16, 17, 18, 19, 20, 21, 22, 23, 24, 25 |
| 37 | 16, 17, 18, 19, 20, 21, 22, 23, 24 |
| 38 | 16, 17, 18, 19, 20, 21, 22, 23 |
| 39 | 16, 17, 18, 19, 20, 21, 22 |
| 40 | 16, 17, 18, 19, 20, 21 |
| 41 | 16, 17, 18, 19, 20 |
| 42 | 16, 17, 18, 19 |
| 43 | 16, 17, 18 |
| 44 | 16, 17 |
| 45 | 16 |
| 47 | 16, 17, 18, 19, 20, 21, 22, 23 |
| 48 | 16, 17, 18, 19, 20, 21, 22 |
| 49 | 16, 17, 18, 19, 20, 21 |
| 50 | 16, 17, 18, 19, 20 |
| 51 | 16, 17, 18, 19 |
| 52 | 16, 17, 18 |
| 53 | 16, 17 |
| 54 | 16 |
| 61 | 16, 17 |
| 62 | 16 |
| 69 | 16, 17, 18, 19, 20, 21, 22, 23, 24 |
| 70 | 16, 17, 18, 19, 20, 21, 22, 23 |
| 71 | 16, 17, 18, 19, 20, 21, 22, 23, 24, 25, 26, 27, 28, 29, 30, 31 |
| 72 | 16, 17, 18, 19, 20, 21, 22, 23, 24, 25, 26, 27, 28, 29, 30 |
| 73 | 16, 17, 18, 19, 20, 21, 22, 23, 24, 25, 26, 27, 28, 29 |
| 74 | 16, 17, 18, 19, 20, 21, 22, 23, 24, 25, 26, 27, 28 |
| 75 | 16, 17, 18, 19, 20, 21, 22, 23, 24, 25, 26, 27 |
| 76 | 16, 17, 18, 19, 20, 21, 22, 23, 24, 25, 26 |
| 77 | 16, 17, 18, 19, 20, 21, 22, 23, 24, 25 |
| 78 | 16, 17, 18, 19, 20, 21, 22, 23, 24 |
| 79 | 16, 17, 18, 19, 20, 21, 22, 23 |
| 80 | 16, 17, 18, 19, 20, 21, 22 |
| 81 | 16, 17, 18, 19, 20, 21 |
| 82 | 16, 17, 18, 19, 20 |
| 83 | 16, 17, 18, 19 |
| 84 | 16, 17, 18 |
| 85 | 16, 17 |
| 86 | 16 |
| 91 | 16, 17 |
| 92 | 16 |
| 95 | 16, 17, 18, 19, 20, 21 |
| 96 | 16, 17, 18, 19, 20 |
| 97 | 16, 17, 18, 19 |
| 98 | 16, 17, 18 |
| 99 | 16, 17 |
| 100 | 16 |
| 104 | 16 |
| 106 | 16, 17, 18, 19, 20, 21, 22, 23, 24, 25, 26, 27, 28, 29, 30, 31, 32 |
| 107 | 16, 17, 18, 19, 20, 21, 22, 23, 24, 25, 26, 27, 28, 29, 30, 31, 32 |
| 108 | 16, 17, 18, 19, 20, 21, 22, 23, 24, 25, 26, 27, 28, 29, 30, 31, 32 |
| 109 | 16, 17, 18, 19, 20, 21, 22, 23, 24, 25, 26, 27, 28, 29, 30, 31, 32 |
| 110 | 16, 17, 18, 19, 20, 21, 22, 23, 24, 25, 26, 27, 28, 29, 30, 31, 32 |
| 111 | 16, 17, 18, 19, 20, 21, 22, 23, 24, 25, 26, 27, 28, 29, 30, 31, 32 |
| 112 | 16, 17, 18, 19, 20, 21, 22, 23, 24, 25, 26, 27, 28, 29, 30, 31, 32 |
| 113 | 16, 17, 18, 19, 20, 21, 22, 23, 24, 25, 26, 27, 28, 29, 30, 31, 32 |
| 114 | 16, 17, 18, 19, 20, 21, 22, 23, 24, 25, 26, 27, 28, 29, 30, 31, 32 |
| 115 | 16, 17, 18, 19, 20, 21, 22, 23, 24, 25, 26, 27, 28, 29, 30, 31 |
| 116 | 16, 17, 18, 19, 20, 21, 22, 23, 24, 25, 26, 27, 28, 29, 30 |
| 117 | 16, 17, 18, 19, 20, 21, 22, 23, 24, 25, 26, 27, 28, 29 |
| 118 | 16, 17, 18, 19, 20, 21, 22, 23, 24, 25, 26, 27, 28 |
| 119 | 16, 17, 18, 19, 20, 21, 22, 23, 24, 25, 26, 27 |
| 120 | 16, 17, 18, 19, 20, 21, 22, 23, 24, 25, 26 |
| 121 | 16, 17, 18, 19, 20, 21, 22, 23, 24, 25 |
| 122 | 16, 17, 18, 19, 20, 21, 22, 23, 24 |
| 123 | 16, 17, 18, 19, 20, 21, 22, 23 |
| 124 | 16, 17, 18, 19, 20, 21, 22 |
| 125 | 16, 17, 18, 19, 20, 21 |
| 126 | 16, 17, 18, 19, 20 |
| 127 | 16, 17, 18, 19 |
| 128 | 16, 17, 18 |
| 129 | 16, 17 |
| 130 | 16 |
| 137 | 16, 17, 18, 19, 20, 21, 22, 23, 24, 25, 26, 27, 28, 29, 30 |
| 138 | 16, 17, 18, 19, 20, 21, 22, 23, 24, 25, 26, 27, 28, 29 |
| 139 | 16, 17, 18, 19, 20, 21, 22, 23, 24, 25, 26, 27, 28 |
| 140 | 16, 17, 18, 19, 20, 21, 22, 23, 24, 25, 26, 27 |
| 141 | 16, 17, 18, 19, 20, 21, 22, 23, 24, 25, 26 |
| 142 | 16, 17, 18, 19, 20, 21, 22, 23, 24, 25 |
| 143 | 16, 17, 18, 19, 20, 21, 22, 23, 24 |
| 144 | 16, 17, 18, 19, 20, 21, 22, 23 |
| 145 | 16, 17, 18, 19, 20, 21, 22 |
| 146 | 16, 17, 18, 19, 20, 21 |
| 147 | 16, 17, 18, 19, 20 |
| 148 | 16, 17, 18, 19 |
| 149 | 16, 17, 18 |
| 150 | 16, 17 |
| 151 | 16, 17, 18, 19, 20, 21, 22, 23, 24, 25, 26, 27, 28, 29, 30, 31, 32 |
| 152 | 16, 17, 18, 19, 20, 21, 22, 23, 24, 25, 26, 27, 28, 29, 30, 31 |
| 153 | 16, 17, 18, 19, 20, 21, 22, 23, 24, 25, 26, 27, 28, 29, 30 |
| 154 | 16, 17, 18, 19, 20, 21, 22, 23, 24, 25, 26, 27, 28, 29 |
| 155 | 16, 17, 18, 19, 20, 21, 22, 23, 24, 25, 26, 27, 28 |
| 156 | 16, 17, 18, 19, 20, 21, 22, 23, 24, 25, 26, 27 |
| 157 | 16, 17, 18, 19, 20, 21, 22, 23, 24, 25, 26 |
| 158 | 16, 17, 18, 19, 20, 21, 22, 23, 24, 25 |
| 159 | 16, 17, 18, 19, 20, 21, 22, 23, 24 |
| 160 | 16, 17, 18, 19, 20, 21, 22, 23 |
| 161 | 16, 17, 18, 19, 20, 21, 22 |
| 162 | 16, 17, 18, 19, 20, 21 |
| 163 | 16, 17, 18, 19, 20 |
| 164 | 16, 17, 18, 19 |
| 165 | 16, 17, 18 |
| 166 | 16, 17 |
| 167 | 16 |
| 171 | 16, 17, 18, 19, 20, 21, 22 |
| 172 | 16, 17, 18, 19, 20, 21 |
| 173 | 16, 17, 18, 19, 20 |
| 174 | 16, 17, 18, 19 |
| 175 | 16, 17, 18 |
| 176 | 16, 17 |
| 177 | 16 |
| 183 | 16, 17, 18, 19, 20, 21, 22, 23, 24, 25, 26, 27 |
| 184 | 16, 17, 18, 19, 20, 21, 22, 23, 24, 25, 26 |
| 185 | 16, 17, 18, 19, 20, 21, 22, 23, 24, 25 |
| 186 | 16, 17, 18, 19, 20, 21, 22, 23, 24 |
| 187 | 16, 17, 18, 19, 20, 21, 22, 23 |
| 188 | 16, 17, 18, 19, 20, 21, 22 |
| 189 | 16, 17, 18, 19, 20, 21 |
| 190 | 16, 17, 18, 19, 20 |
| 191 | 16, 17, 18, 19 |
| 192 | 16, 17, 18 |
| 193 | 16, 17 |
| 194 | 16 |
| 201 | 16, 17, 18 |
| 202 | 16, 17 |
| 203 | 16, 17, 18, 19, 20, 21, 22, 23, 24, 25, 26, 27, 28, 29, 30, 31, 32 |
| 204 | 16, 17, 18, 19, 20, 21, 22, 23, 24, 25, 26, 27, 28, 29, 30, 31, 32 |
| 205 | 16, 17, 18, 19, 20, 21, 22, 23, 24, 25, 26, 27, 28, 29, 30, 31, 32 |
| 206 | 16, 17, 18, 19, 20, 21, 22, 23, 24, 25, 26, 27, 28, 29, 30, 31, 32 |
| 207 | 16, 17, 18, 19, 20, 21, 22, 23, 24, 25, 26, 27, 28, 29, 30, 31, 32 |
| 208 | 16, 17, 18, 19, 20, 21, 22, 23, 24, 25, 26, 27, 28, 29, 30, 31, 32 |
| 209 | 16, 17, 18, 19, 20, 21, 22, 23, 24, 25, 26, 27, 28, 29, 30, 31, 32 |
| 210 | 16, 17, 18, 19, 20, 21, 22, 23, 24, 25, 26, 27, 28, 29, 30, 31, 32 |
| 211 | 16, 17, 18, 19, 20, 21, 22, 23, 24, 25, 26, 27, 28, 29, 30, 31, 32 |
| 212 | 16, 17, 18, 19, 20, 21, 22, 23, 24, 25, 26, 27, 28, 29, 30, 31, 32 |
| 213 | 16, 17, 18, 19, 20, 21, 22, 23, 24, 25, 26, 27, 28, 29, 30, 31, 32 |
| 214 | 16, 17, 18, 19, 20, 21, 22, 23, 24, 25, 26, 27, 28, 29, 30, 31, 32 |
| 215 | 16, 17, 18, 19, 20, 21, 22, 23, 24, 25, 26, 27, 28, 29, 30, 31, 32 |
| 216 | 16, 17, 18, 19, 20, 21, 22, 23, 24, 25, 26, 27, 28, 29, 30, 31, 32 |
| 217 | 16, 17, 18, 19, 20, 21, 22, 23, 24, 25, 26, 27, 28, 29, 30, 31, 32 |
| 218 | 16, 17, 18, 19, 20, 21, 22, 23, 24, 25, 26, 27, 28, 29, 30, 31 |
| 219 | 16, 17, 18, 19, 20, 21, 22, 23, 24, 25, 26, 27, 28, 29, 30 |
| 220 | 1617, 18, 19, 20, 21, 22, 23, 24, 25, 26, 27, 28, 29 |
| 221 | 16, 17, 18, 19, 20, 21, 22, 23, 24, 25, 26, 27, 28 |
| 222 | 16, 17, 18, 19, 20, 21, 22, 23, 24, 25, 26, 27 |
| 223 | 16, 17, 18, 19, 20, 21, 22, 23, 24, 25, 26 |
| 224 | 16, 17, 18, 19, 20, 21, 22, 23, 24, 25 |
| 225 | 16, 17, 18, 19, 20, 21, 22, 23, 24 |
| 226 | 16, 17, 18, 19, 20, 21, 22, 23 |
| 227 | 16, 17, 18, 19, 20, 21, 22 |
| 228 | 16, 17, 18, 19, 20, 21 |
| 229 | 16, 17, 18, 19, 20 |
| 230 | 16, 17, 18, 19 |
| 231 | 16, 17, 18 |
| 232 | 16, 17 |
| 233 | 16 |
| 238 | 16, 17, 18, 19, 20, 21, 22, 23, 24, 25, 26, 27, 28, 29, 30, 31 |
| 239 | 16, 17, 18, 19, 20, 21, 22, 23, 24, 25, 26, 27, 28, 29, 30 |
| 240 | 16, 17, 18, 19, 20, 21, 22, 23, 24, 25, 26, 27, 28, 29 |
| 241 | 16, 17, 18, 19, 20, 21, 22, 23, 24, 25, 26, 27, 28 |
| 242 | 16, 17, 18, 19, 20, 21, 22, 23, 24, 25, 26, 27 |
| 243 | 16, 17, 18, 19, 20, 21, 22, 23, 24, 25, 26 |
| 244 | 16, 17, 18, 19, 20, 21, 22, 23, 24, 25 |
| 245 | 16, 17, 18, 19, 20, 21, 22, 23, 24 |
| 246 | 16, 17, 18, 19, 20, 21, 22, 23 |
| 247 | 16, 17, 18, 19, 20, 21, 22 |
| 248 | 16, 17, 18, 19, 20, 21 |
| 249 | 16, 17, 18, 19, 20 |
| 250 | 16, 17, 18, 19 |
| 251 | 16, 17, 18 |
| 252 | 16, 17 |
| 253 | 16 |

Prototype Oligonucleotides:

| Sequence | Trivial | Starting | |
|---|---|---|---|
| ID No. | Name | Position* | 5′-->3′ Sequence |
| 25 | OL(1)CLU35 | 15 | TGCCCATCCC CTCTAAGCCG |
| 26 | OL(2)CLU35 | 32 | TGCCAGGCCC TCCACACTGC |
| 27 | OL(3)CLU35 | 39 | CTTGCTCTGC CAGGCCCTCC |
| 28 | OL(4)CLU35 | 96 | CGCAGTGGCC CGAGGAGCAG |
| 29 | OL(5)CLU35 | 173 | GCCGGGCTGC CTGTGCATTC |
| 30 | OL(6)CLU35 | 183 | GGAGACTTGG GCCGGGCTGC |
| 31 | OL(7)CLU35 | 223 | GCAGCCAGCC CTTCACACCG |
| 32 | OL(8)CLU35 | 238 | GACGCGGAGG CAACAGCAGC |

*Because this is antisense, the position numbers must be read right to left; the starting and ending positions are defined according to the NCBI Reference Sequence with the first base of the sense strand being base number one.

TABLE 4C
Gene: Clusterin (CLU36)
GenBank or NCBI Reference Sequence: None specifically for CLU36 but the sequence for the unique exon
1 of CLU36 appears in Genbank sequence M63376 and encompasses positions 4374 through 4513. The
complete CLU36 sequence appears in the reference.
Reference: Andersen et al., Mol Cell Proteomics 6: 1039, 2007 with CLU36 sequence provided in
associated supplemental material.
HOT-SPOT 1. Range of bases included: positions 4387-4438*
Antisense Strand Sequence:
SEQ ID NO: 33: CAGCAGGCCC ATCTGGACAC TCAGTAGCAC ATTAACAAGG AGTGCCTCTC A
Note: Size variants for this Hot Spot were selected as targets for conventional antisense oligos
with steric hindrance function against the translational start site in exon 1.

| Nucleotide Starting | Size Variants |
|---|---|
| Position* | (Number of bases in the oligomer) |
| 4387 | 22, 23, 24 |
| 4388 | 21, 22, 23 |
| 4389 | 20, 21, 22 |
| 4390 | 19, 20, 21 |
| 4391 | 18, 19, 20 |
| 4392 | 17, 18, 19 |
| 4393 | 16, 17, 18 |
| 4394 | 16, 17 |
| 4395 | 16, 17, 18, 19, 20, 21, 22, 23, 24, 25, 26, 27, 28, 29, 30, 31, 32 |
| 4396 | 16, 17, 18, 19, 20, 21, 22, 23, 24, 25, 26, 27, 28, 29, 30, 31, 32 |
| 4397 | 16, 17, 18, 19, 20, 21, 22, 23, 24, 25, 26, 27, 28, 29, 30, 31, 32 |
| 4398 | 16, 17, 18, 19, 20, 21, 22, 23, 24, 25, 26, 27, 28, 29, 30, 31, 32 |
| 4399 | 16, 17, 18, 19, 20, 21, 22, 23, 24, 25, 26, 27, 28, 29, 30, 31, 32 |
| 4400 | 16, 17, 18, 19, 20, 21, 22, 23, 24, 25, 26, 27, 28, 29, 30, 31, 32 |
| 4401 | 16, 17, 18, 19, 20, 21, 22, 23, 24, 25, 26, 27, 28, 29, 30, 31, 32 |
| 4402 | 16, 17, 18, 19, 20, 21, 22, 23, 24, 25, 26, 27, 28, 29, 30, 31, 32 |
| 4403 | 16, 17, 18, 19, 20, 21, 22, 23, 24, 25, 26, 27, 28, 29, 30, 31, 32 |
| 4404 | 16, 17, 18, 19, 20, 21, 22, 23, 24, 25, 26, 27, 28, 29, 30, 31, 32 |
| 4405 | 16, 17, 18, 19, 20, 21, 22, 23, 24, 25, 26, 27, 28, 29, 30, 31, 32 |
| 4406 | 16, 17, 18, 19, 20, 21, 22, 23, 24, 25, 26, 27, 28, 29, 30, 31, 32 |
| 4407 | 16, 17, 18, 19, 20, 21, 22, 23, 24, 25, 26, 27, 28, 29, 30, 31 |
| 4408 | 16, 17, 18, 19, 20, 21, 22, 23, 24, 25, 26, 27, 28, 29, 30 |

Prototype Oligonucleotides:

| Sequence | Trivial | Starting | |
|---|---|---|---|
| ID No. | Name | Position* | 5′-->3′ Sequence |
| 34 | A(1)CLU36 | 4388 | CACATTAACA AGGAGTGCCT CTC |
| 35 | A(2)CLU36 | 4404 | TCTGGACACT CAGTAGCACA TTA |

Gene: Clusterin (CLU36)
GenBank or NCBI Reference Sequence: None
The CLU36 sequence used for this analysis appears in the reference.
Reference: Andersen et al., Mol Cell Proteomics 6: 1039, 2007 with CLU36 sequence provided in
associated supplemental material.
HOT-SPOT 2. Range of bases included: positions 108-148*
Antisense Strand Sequence:
SEQ ID NO:36: GTCTTTGCAC GCCTCCATTT GCCATCACAG AACCAACAGG ACGATGGG
Note: Size variants for this Hot Spot were selected as targets for conventional antisense oligos
with steric hindrance function against the alternative primary translational start site in exon 1.

| Nucleotide Starting | Size Variants |
|---|---|
| Position* | (Number of bases in the oligomer) |
| 108 | 32 |
| 109 | 31, 32 |
| 110 | 30, 31, 32 |
| 111 | 29, 30, 31, 32 |
| 112 | 28, 29, 30, 31, 32 |
| 113 | 27, 28, 29, 30, 31, 32 |
| 114 | 26, 27, 28, 29, 30, 31, 32 |
| 115 | 25, 26, 27, 28, 29, 30, 31, 32 |
| 116 | 24, 25, 26, 27, 28, 29, 30, 31, 32 |
| 117 | 23, 24, 25, 26, 27, 28, 29, 30, 31, 32 |
| 118 | 22, 23, 24, 25, 26, 27, 28, 29, 30, 31 |
| 119 | 21, 22, 23, 24, 25, 26, 27, 28, 29, 30 |
| 120 | 20, 21, 22, 23, 24, 25, 26, 27, 28, 29 |
| 121 | 19, 20, 21, 22, 23, 24, 25, 26, 27, 28 |
| 122 | 18, 19, 20, 21, 22, 23, 24, 25, 26, 27 |
| 123 | 17, 18, 19, 20, 21, 22, 23, 24, 25, 26 |
| 124 | 16, 17, 18, 19, 20, 21, 22, 23, 24, 25 |
| 125 | 16, 17, 18, 19, 20, 21, 22, 23, 24 |
| 126 | 16, 17, 18, 19, 20, 21, 22, 23 |
| 127 | 16, 17, 18, 19, 20, 21, 22 |
| 128 | 16, 17, 18, 19, 20, 21 |
| 129 | 16, 17, 18, 19, 20 |
| 130 | 16, 17, 18, 19 |
| 131 | 16, 17, 18 |
| 132 | 16, 17 |
| 133 | 16 |

Prototype Oligonucleotides:

| Sequence | Trivial | Starting | |
|---|---|---|---|
| ID No. | Name | Position* | 5′-->3′ Sequence |
| 37 | A(3)CLU36 | 125 | CGCCTCCATT TGCCATCACA GA |
| 38 | A(4)CLU36 | 129 | CACGCCTCCA TTTGCCATCA |

The following oligos have an RNase H mechanism of action
Gene: Clusterin (CLU36)
GenBank or NCBI Reference Sequence: None specifically for CLU36 but the sequence for the unique exon
1 of CLU36 appears in Genbank sequence M63376 and encompasses positions 4374 through 4513. The
complete CLU36 sequence appears in the reference.
Reference: Andersen et al., Mol Cell Proteomics 6: 1039, 2007 with CLU36 sequence provided in
associated supplemental material.
HOT-SPOT 3. Range of bases included: positions 4374-4513*
Antisense Strand Sequence:
SEQ ID NO: 39:
CCATTTGCCA TCACAGAACC AACAGGACGA TGGGTTCCCC TTCCTGAAAT GGTTCACATC
CAAAGCCCGC TCAGCCCAGC AGGCCCATCT GGACACTCAG TAGCACATTA ACAAGGAGTG
CCTCTCATGA GAAGTAATCA

| Nucleotide Starting | Size Variants |
|---|---|
| Position* | (Number of bases in the oligomer) |
| 4374 | 16, 17, 18, 19, 20, 21, 22, 23, 24, 25, 26, 27, 28, 29, 30, 31, 32 |
| 4375 | 16, 17, 18, 19, 20, 21, 22, 23, 24, 25, 26, 27, 28, 29, 30, 31, 32 |
| 4376 | 16, 17, 18, 19, 20, 21, 22, 23, 24, 25, 26, 27, 28, 29, 30, 31, 32 |
| 4377 | 16, 17, 18, 19, 20, 21, 22, 23, 24, 25, 26, 27, 28, 29, 30, 31, 32 |
| 4378 | 16, 17, 18, 19, 20, 21, 22, 23, 24, 25, 26, 27, 28, 29, 30, 31, 32 |
| 4379 | 16, 17, 18, 19, 20, 21, 22, 23, 24, 25, 26, 27, 28, 29, 30, 31, 32 |
| 4380 | 16, 17, 18, 19, 20, 21, 22, 23, 24, 25, 26, 27, 28, 29, 30, 31 |
| 4381 | 16, 17, 18, 19, 20, 21, 22, 23, 24, 25, 26, 27, 28, 29, 30 |
| 4382 | 16, 17, 18, 19, 20, 21, 22, 23, 24, 25, 26, 27, 28, 29 |
| 4383 | 16, 17, 18, 19, 20, 21, 22, 23, 24, 25, 26, 27, 28 |
| 4384 | 16, 17, 18, 19, 20, 21, 22, 23, 24, 25, 26, 27 |
| 4385 | 16, 17, 18, 19, 20, 21, 22, 23, 24, 25, 26 |
| 4386 | 16, 17, 18, 19, 20, 21, 22, 23, 24, 25 |
| 4387 | 16, 17, 18, 19, 20, 21, 22, 23, 24 |
| 4388 | 16, 17, 18, 19, 20, 21, 22, 23 |
| 4389 | 16, 17, 18, 19, 20, 21, 22 |
| 4390 | 16, 17, 18, 19, 20, 21 |
| 4391 | 16, 17, 18, 19, 20 |
| 4392 | 16, 17, 18, 19 |
| 4393 | 16, 17, 18 |
| 4394 | 16, 17, 18, 19, 20, 21, 22, 23, 24, 25, 26, 27, 28, 29, 30, 31, 32 |
| 4395 | 16, 17, 18, 19, 20, 21, 22, 23, 24, 25, 26, 27, 28, 29, 30, 31, 32 |
| 4396 | 16, 17, 18, 19, 20, 21, 22, 23, 24, 25, 26, 27, 28, 29, 30, 31, 32 |
| 4397 | 16, 17, 18, 19, 20, 21, 22, 23, 24, 25, 26, 27, 28, 29, 30, 31, 32 |
| 4398 | 16, 17, 18, 19, 20, 21, 22, 23, 24, 25, 26, 27, 28, 29, 30, 31, 32 |
| 4399 | 16, 17, 18, 19, 20, 21, 22, 23, 24, 25, 26, 27, 28, 29, 30, 31, 32 |
| 4400 | 16, 17, 18, 19, 20, 21, 22, 23, 24, 25, 26, 27, 28, 29, 30, 31, 32 |
| 4401 | 16, 17, 18, 19, 20, 21, 22, 23, 24, 25, 26, 27, 28, 29, 30, 31, 32 |
| 4402 | 16, 17, 18, 19, 20, 21, 22, 23, 24, 25, 26, 27, 28, 29, 30, 31, 32 |
| 4403 | 16, 17, 18, 19, 20, 21, 22, 23, 24, 25, 26, 27, 28, 29, 30, 31, 32 |
| 4404 | 16, 17, 18, 19, 20, 21, 22, 23, 24, 25, 26, 27, 28, 29, 30, 31, 32 |
| 4405 | 16, 17, 18, 19, 20, 21, 22, 23, 24, 25, 26, 27, 28, 29, 30, 31, 32 |
| 4406 | 16, 17, 18, 19, 20, 21, 22, 23, 24, 25, 26, 27, 28, 29, 30, 31, 32 |
| 4407 | 16, 17, 18, 19, 20, 21, 22, 23, 24, 25, 26, 27, 28, 29, 30, 31 |
| 4408 | 16, 17, 18, 19, 20, 21, 22, 23, 24, 25, 26, 27, 28, 29, 30 |
| 4409 | 16, 17, 18, 19, 20, 21, 22, 23, 24, 25, 26, 27, 28, 29 |
| 4410 | 16, 17, 18, 19, 20, 21, 22, 23, 24, 25, 26, 27, 28 |
| 4411 | 16, 17, 18, 19, 20, 21, 22, 23, 24, 25, 26, 27 |
| 4412 | 16, 17, 18, 19, 20, 21, 22, 23, 24, 25, 26 |
| 4413 | 16, 17, 18, 19, 20, 21, 22, 23, 24, 25 |
| 4414 | 16, 17, 18, 19, 20, 21, 22, 23, 24 |
| 4415 | 16, 17, 18, 19, 20, 21, 22, 23 |
| 4416 | 16, 17, 18, 19, 20, 21, 22 |
| 4417 | 16, 17, 18, 19, 20, 21 |
| 4418 | 16, 17, 18, 19, 20 |
| 4419 | 16, 17, 18, 19 |
| 4420 | 16, 17, 18 |
| 4421 | 16, 17 |
| 4422 | 16 |
| 4424 | 16 |
| 4435 | 16, 17, 18, 19, 20, 21, 22, 23, 24, 25, 26, 27, 28, 29 |
| 4436 | 16, 17, 18, 19, 20, 21, 22, 23, 24, 25, 26, 27, 28 |
| 4437 | 16, 17, 18, 19, 20, 21, 22, 23, 24, 25, 26, 27 |
| 4438 | 16, 17, 18, 19, 20, 21, 22, 23, 24, 25, 26 |
| 4439 | 16, 17, 18, 19, 20, 21, 22, 23, 24, 25 |
| 4440 | 16, 17, 18, 19, 20, 21, 22, 23, 24 |
| 4441 | 16, 17, 18, 19, 20, 21, 22, 23 |
| 4442 | 16, 17, 18, 19, 20, 21, 22 |
| 4443 | 16, 17, 18, 19, 20, 21 |
| 4444 | 16, 17, 18, 19, 20 |
| 4445 | 16, 17, 18, 19 |
| 4446 | 16, 17, 18 |
| 4447 | 16, 17 |
| 4448 | 16 |
| 4453 | 16, 17, 18, 19, 20, 21, 22, 23, 24, 25, 26, 27, 28, 29, 30, 31, 32 |
| 4454 | 16, 17, 18, 19, 20, 21, 22, 23, 24, 25, 26, 27, 28, 29, 30, 31, 32 |
| 4455 | 16, 17, 18, 19, 20, 21, 22, 23, 24, 25, 26, 27, 28, 29, 30, 31, 32 |
| 4456 | 16, 17, 18, 19, 20, 21, 22, 23, 24, 25, 26, 27, 28, 29, 30, 31, 32 |
| 4457 | 16, 17, 18, 19, 20, 21, 22, 23, 24, 25, 26, 27, 28, 29, 30, 31, 32 |
| 4458 | 16, 17, 18, 19, 20, 21, 22, 23, 24, 25, 26, 27, 28, 29, 30, 31 |
| 4459 | 16, 17, 18, 19, 20, 21, 22, 23, 24, 25, 26, 27, 28, 29, 30 |
| 4460 | 16, 17, 18, 19, 20, 21, 22, 23, 24, 25, 26, 27, 28, 29 |
| 4461 | 16, 17, 18, 19, 20, 21, 22, 23, 24, 25, 26, 27, 28 |
| 4462 | 16, 17, 18, 19, 20, 21, 22, 23, 24, 25, 26, 27 |
| 4463 | 16, 17, 18, 19, 20, 21, 22, 23, 24, 25, 26 |
| 4464 | 16, 17, 18, 19, 20, 21, 22, 23, 24, 25 |
| 4465 | 16, 17, 18, 19, 20, 21, 22, 23, 24 |
| 4466 | 16, 17, 18, 19, 20, 21, 22, 23 |
| 4467 | 16, 17, 18, 19, 20, 21, 22 |
| 4468 | 16, 17, 18, 19, 20, 21 |
| 4469 | 16, 17, 18, 19, 20 |
| 4470 | 16, 17, 18, 19 |
| 4471 | 16, 17, 18 |
| 4472 | 16, 17 |
| 4473 | 16 |
| 4478 | 16, 17 |
| 4479 | 16 |
| 4482 | 16, 17, 18, 19, 20, 21, 22, 23, 24, 25, 26, 27, 28, 29, 30, 31 |
| 4483 | 16, 17, 18, 19, 20, 21, 22, 23, 24, 25, 26, 27, 28, 29, 30 |
| 4484 | 16, 17, 18, 19, 20, 21, 22, 23, 24, 25, 26, 27, 28, 29 |
| 4485 | 16, 17, 18, 19, 20, 21, 22, 23, 24, 25, 26, 27, 28 |
| 4486 | 16, 17, 18, 19, 20, 21, 22, 23, 24, 25, 26, 27 |
| 4487 | 16, 17, 18, 19, 20, 21, 22, 23, 24, 25, 26 |
| 4488 | 16, 17, 18, 19, 20, 21, 22, 23, 24, 25 |
| 4489 | 16, 17, 18, 19, 20, 21, 22, 23, 24 |
| 4490 | 16, 17, 18, 19, 20, 21, 22, 23 |
| 4491 | 16, 17, 18, 19, 20, 21, 22 |
| 4492 | 16, 17, 18, 19, 20, 21 |
| 4493 | 16, 17, 18, 19, 20 |
| 4494 | 16, 17, 18, 19 |
| 4495 | 16, 17, 18 |
| 4496 | 16, 17 |
| 4497 | 16 |

Prototype Oligonucleotides:

| Sequence | Trivial | Starting | |
|---|---|---|---|
| ID No. | Name | Position* | 5′-->3′ Sequence |
| 40 | OL(1)CLU36 | 4418 | CAGCAGGCCC ATCTGGACAC |
| 41 | OL(2)CLU36 | 4436 | TCCAAAGCCC GCTCAGCCCA |
| 42 | OL(3)CLU36 | 4444 | GGTTCACATC CAAAGCCCGC |
| 43 | OL(4)CLU36 | 4467 | CGATGGGTTC CCCTTCCTGA |

*Because this is antisense, the position numbers must be read right to left; the starting and ending positions are defined according to the NCBI Reference Sequence with the first base of the sense strand being base number one.

TABLE 4D
The Human SGP2 Gene
Gene: SGP2 GenBank: HUMSGLY/M74816
References: Danik et al., Proc. Natl. Acad. Sci. 88; 8577 (1991).
HOT-SPOT 1. Range of bases included: positions 480-527*
Antisense Strand Sequence:
SEQ ID NO: 44: GTACGGAGAG AAGGGCATCA AGCTGCGGAC GATGCGGGAC
TTGGGAAA

| Nucleotide Starting | Size Variants |
|---|---|
| Position* | (Number of bases in the oligomer) |
| 480 | 16, 17, 18, 19, 20, 21, 22, 23, 24, 25, 26, 27 |
| 481 | 16, 17, 18, 19, 20, 21, 22, 23, 24, 25, 26, 27 |
| 482 | 16, 17, 18, 19, 20, 21, 22, 23, 24, 25, 26, 27 |
| 483 | 16, 17, 18, 19, 20, 21, 22, 23, 24, 25, 26, 27 |
| 484 | 16, 17, 18, 19, 20, 21, 22, 23, 24, 25, 26, 27 |
| 485 | 16, 17, 18, 19, 20, 21, 22, 23, 24, 25, 26, 27 |
| 486 | 16, 17, 18, 19, 20, 21, 22, 23, 24, 25, 26, 27 |
| 487 | 16, 17, 18, 19, 20, 21, 22, 23, 24, 25, 26 |
| 488 | 16, 17, 18, 19, 20, 21, 22, 23, 24, 25 |
| 489 | 16, 17, 18, 19, 20, 21, 22, 23, 24 |
| 490 | 16, 17, 18, 19, 20, 21, 22, 23 |
| 491 | 16, 17, 18, 19, 20, 21, 22 |
| 492 | 16, 17, 18, 19, 20, 21 |
| 493 | 16, 17, 18, 19, 20 |
| 494 | 16, 17, 18, 19, 20, 21, 22, 23, 24, 25, 26, 27 |
| 495 | 16, 17, 18, 19, 20, 21, 22, 23, 24, 25, 26, 27 |
| 496 | 16, 17, 18, 19, 20, 21, 22, 23, 24, 25, 26, 27 |
| 497 | 16, 17, 18, 19, 20, 21, 22, 23, 24, 25, 26, 27 |
| 498 | 16, 17, 18, 19, 20, 21, 22, 23, 24, 25, 26, 27 |
| 499 | 16, 17, 18, 19, 20, 21, 22, 23, 24, 25, 26, 27 |
| 500 | 16, 17, 18, 19, 20, 21, 22, 23, 24, 25, 26, 27 |
| 501 | 16, 17, 18, 19, 20, 21, 22, 23, 24, 25, 26, 27 |

Prototype Oligonucleotides:

| Sequence | Trivial | Starting | |
|---|---|---|---|
| ID No. | Name | Position* | 5′-->3′ Sequence |
| 45 | OL(1)SGP2 | 483 | TGCGGACGAT GCGGGACTTG GG |
| 46 | OL(2)SGP2 | 494 | GGGCATCAAG CTGCGGACGA TG |

*Because this is antisense, the position numbers must be read right to left; the starting and ending
positions are defined according to the GenBank sequence with the first base of the sense strand
being base number one.
Gene: SGP2
GenBank: HUMSGLY/M74816 References: Danik et al., Proc. Natl. Acad. Sci. 88; 8577 (1991).
HOT-SPOT 2. Range of bases included: positions 606-647*
Antisense Strand Sequence:
SEQ ID NO: 47: TATGAATTCT GTTGGCGGGT GCTGGAAGGC CGGGCTGTGG AA

| Nucleotide Starting | Size Variants |
|---|---|
| Position* | (Number of bases in the oligomer) |
| 606 | 16, 17, 18, 19, 20, 21, 22, 23, 24, 25, 26 |
| 607 | 16, 17, 18, 19, 20, 21, 22, 23, 24, 25 |
| 608 | 16, 17, 18, 19, 20, 21, 22, 23, 24 |
| 609 | 16, 17, 18, 19, 20, 21, 22, 23 |
| 610 | 16, 17, 18, 19, 20, 21, 22 |
| 611 | 16, 17, 18, 19, 20, 21 |
| 612 | 16, 17, 18, 19, 20 |
| 613 | 16, 17, 18, 19 |
| 614 | 16, 17, 18 |
| 615 | 16, 17 |
| 616 | 16 |
| 617 | |
| 618 | 16, 17, 18, 19, 20, 21, 22, 23, 24, 25, 26, 27 |
| 619 | 16, 17, 18, 19, 20, 21, 22, 23, 24, 25, 26, 27 |
| 620 | 16, 17, 18, 19, 20, 21, 22, 23, 24, 25, 26, 27 |
| 621 | 16, 17, 18, 19, 20, 21, 22, 23, 24, 25, 26, 27 |

Prototype Oligonucleotides:

| Sequence | Trivial | Starting | |
|---|---|---|---|
| ID No. | Name | Position* | 5′-->3′ Sequence |
| 48 | OL(3)SGP2 | 607 | TGCTGGAAGG CCGGGCTGTG GA |
| 49 | OL(4)SGP2 | 618 | TCTGTTGGCG GGTGCTGGAA GGC |

*Because this is antisense, the position numbers must be read right to left; the starting and ending
positions are defined according to the GenBank sequence with the first base of the sense strand
being base number one.
Gene: SGP2
GenBank: HUMSGLY/M74816
References: Danik et al., Proc. Natl. Acad. Sci. 88; 8577 (1991).
HOT-SPOT 3. Range of bases included: positions 919-960*
Antisense Strand Sequence:
SEQ ID NO: 50: CTTCGCCTTG CGTGAGGTTT GCCAGCCGGG ACACCCAGTT AA

| Nucleotide Starting | Size Variants |
|---|---|
| Position* | (Number of bases in the oligomer) |
| 919 | 16, 17, 18, 19, 20, 21, 22, 23, 24, 25, 26, 27 |
| 920 | 16, 17, 18, 19, 20, 21, 22, 23, 24, 25, 26, 27 |
| 921 | 16, 17, 18, 19, 20, 21, 22, 23, 24, 25, 26, 27 |
| 922 | 16, 17, 18, 19, 20, 21, 22, 23, 24, 25, 26, 27 |
| 923 | 16, 17, 18, 19, 20, 21, 22, 23, 24, 25, 26, 27 |
| 924 | 16, 17, 18, 19, 20, 21, 22, 23, 24, 25, 26, 27 |
| 925 | 16, 17, 18, 19, 20, 21, 22, 23, 24, 25, 26, 27 |
| 926 | 16, 17, 18, 19, 20, 21, 22, 23, 24, 25, 26, 27 |
| 927 | 16, 17, 18, 19, 20, 21, 22, 23, 24, 25, 26, 27 |
| 928 | 16, 17, 18, 19, 20, 21, 22, 23, 24, 25, 26, 27 |
| 929 | 16, 17, 18, 19, 20, 21, 22, 23, 24, 25, 26, 27 |
| 930 | 16, 17, 18, 19, 20, 21, 22, 23, 24, 25, 26, 27 |
| 931 | 16, 17, 18, 19, 20, 21, 22, 23, 24, 25, 26, 27 |
| 932 | 16, 17, 18, 19, 20, 21, 22, 23, 24, 25, 26, 27 |
| 933 | 16, 17, 18, 19, 20, 21, 22, 23, 24, 25, 26, 27 |
| 934 | 16, 17, 18, 19, 20, 21, 22, 23, 24, 25, 26, 27 |

Prototype Oligonucleotides:

| Sequence | Trivial | Starting | |
|---|---|---|---|
| ID No. | Name | Position* | 5′-->3′ Sequence |
| 51 | OL(5)SGP2 | 924 | GGTTTGCCAG CCGGGACACC CA |
| 52 | OL(6)SGP2 | 931 | TGCGTGAGGT TTGCCAGCCG GG |

*Because this is antisense, the position numbers must be read right to left; the starting and ending
positions are defined according to the GenBank sequence with the first base of the sense strand
being base number one.
Gene: SGP2
GenBank: HUMSGLY/M74816
References: Danik et al., Proc. Natl. Acad. Sci. 88; 8577 (1991).
HOT-SPOT 4. Range of bases included: positions 1294-1316*
Antisense Strand Sequence: SEQ ID NO:53: GGGAGAGGCT GGGCGGAGTT GGG

| Nucleotide Starting | Size Variants |
|---|---|
| Position* | (Number of bases in the oligomer) |
| 1294 | 16, 17, 18, 19, 20, 21, 22, 23 |
| 1295 | 16, 17, 18, 19, 20, 21, 22 |
| 1296 | 16, 17, 18, 19, 20, 21 |
| 1297 | 16, 17, 18, 19, 20 |
| 1298 | 16, 17, 18, 19 |
| 1299 | 16, 17, 18 |
| 1300 | 16, 17 |
| 1301 | 16 |

Prototype Oligonucleotides:

| Sequence | Trivial | Starting | |
|---|---|---|---|
| ID No. | Name | Position* | 5′-->3′ Sequence |
| 54 | OL(7)SGP2 | 1294 | GGGAGAGGCT GGGCGGAGTT GGG |

*Became this is antisense, the position numbers must be read right to left; the starting and ending
positions are defined according to the GenBank sequence with the first base of the sense strand
being base number one.
Gene: SGP2
GenBank: HUMSGLY/M74816
References: Danik et al., Proc. Natl. Acad. Sci. 88; 8577 (1991).
HOT-SPOT 5. Range of bases included: positions 226-258*
Antisense Strand Sequence:
SEQ ID NO: 55: GGTTCAGGAA CTCCTCAAGC TGGCGGCCAA CCA

| Nucleotide Starting | Size Variants |
|---|---|
| Position* | (Number of bases in the oligomer) |
| 226 | 16, 17, 18, 19, 20, 21, 22, 23, 24, 25, 26, 27 |
| 227 | 16, 17, 18, 19, 20, 21, 22, 23, 24, 25, 26, 27 |
| 228 | 16, 17, 18, 19, 20, 21, 22, 23, 24, 25, 26, 27 |
| 229 | 16, 17, 18, 19, 20, 21, 22, 23, 24, 25, 26, 27 |
| 230 | 16, 17, 18, 19, 20, 21, 22, 23, 24, 25, 26, 27 |
| 231 | 16, 17, 18, 19, 20, 21, 22, 23, 24, 25, 26, 27 |
| 232 | 16, 17, 18, 19, 20, 21, 22, 23, 24, 25, 26, 27 |

Prototype Oligonucleotides:

| Sequence | Trivial | Starting | 5′-->3′ Sequence |
|---|---|---|---|
| ID No. | Name | Position* | |
| 56 | OL(8)SGP2 | 230 | GGAACTCCTC AAGCTGGCGG CCA |

*Because this is antisense, the position numbers must be read right to left; the starting and ending
positions are defined according to the GenBank sequence with the first base of the sense strand
being base number one.
Gene: SGP2
GenBank: HUMSGLY/M74816
References: Danik et al., Proc. Natl. Acad. Sci. 88; 8577 (1991).
HOT-SPOT 6. Range of bases included: positions 348-385*
Antisense Strand Sequence:
SEQ ID NO: 57: TCTATGATGC TGGACGCGCG GCTGAAGTGG TCCTGCAT

| Nucleotide Starting | Size Variants |
|---|---|
| Position* | (Number of bases in the oligomer) |
| 348 | 16, 17, 18, 19, 20, 21, 22, 23, 24, 25, 26, 27 |
| 349 | 16, 17, 18, 19, 20, 21, 22, 23, 24, 25, 26, 27 |
| 350 | 16, 17, 18, 19, 20, 21, 22, 23, 24, 25, 26, 27 |
| 351 | 16, 17, 18, 19, 20, 21, 22, 23, 24, 25, 26, 27 |
| 352 | 16, 17, 18, 19, 20, 21, 22, 23, 24, 25, 26, 27 |
| 353 | 16, 17, 18, 19, 20, 21, 22, 23, 24, 25, 26, 27 |
| 354 | 16, 17, 18, 19, 20, 21, 22, 23, 24, 25, 26, 27 |
| 355 | 16, 17, 18, 19, 20, 21, 22, 23, 24, 25, 26, 27 |
| 356 | 16, 17, 18, 19, 20, 21, 22, 23, 24, 25, 26, 27 |
| 357 | 16, 17, 18, 19, 20, 21, 22, 23, 24, 25, 26, 27 |
| 358 | 16, 17, 18, 19, 20, 21, 22, 23, 24, 25, 26, 27 |
| 359 | 16, 17, 18, 19, 20, 21, 22, 23, 24, 25, 26, 27 |
| 360 | 16, 17, 18, 19, 20, 21, 22, 23, 24, 25, 26, 27 |
| 361 | 16, 17, 18, 19, 20, 21, 22, 23, 24, 25, 26 |
| 362 | 16, 17, 18, 19, 20, 21, 22, 23, 24, 25 |
| 363 | 16, 17, 18, 19, 20, 21, 22, 23, 24 |
| 364 | 16, 17, 18, 19, 20, 21, 22, 23 |
| 365 | 16, 17, 18, 19, 20, 21, 22 |
| 366 | 16, 17, 18, 19, 20, 21 |
| 367 | 16, 17, 18, 19, 20 |
| 368 | 16, 17, 18, 19 |
| 369 | 16, 17, 18 |
| 370 | 16, 17 |

Prototype Oligonucleotides:

| Sequence | Trivial | Starting | |
|---|---|---|---|
| ID No. | Name | Position* | 5′-->3′ Sequence |
| 58 | OL(9)SGP2 | 356 | GCTGGACGCG CGGCTGAAGT GG |
| 59 | OL(10)SGP2 | 349 | CGCGCGGCTG AAGTGGTCCT GCA |

*Because this is antisense, the position numbers must be read right to left; the starting and ending
positions are defined according to the GenBank sequence with the first base of the sense strand
being base number one.
Gene: SGP2
GenBank: HUMSGLY/M74816
References: Danik et al., Proc. Natl. Acad. Sci. 88; 8577 (1991).
HOT-SPOT 7. Range of bases included: positions 691-728*
Antisense Strand Sequence:
SEQ ID NO: 60: ACACTGGTCC TTCATCCGCA GGCAGCCCGT GGAGTTGT

| Nucleotide Starting | Size Variants |
|---|---|
| Position* | (Number of bases in the oligomer) |
| 691 | 16, 17, 18, 19, 20, 21, 22, 23, 24, 25, 26, 27 |
| 692 | 16, 17, 18, 19, 20, 21, 22, 23, 24, 25, 26, 27 |
| 693 | 16, 17, 18, 19, 20, 21, 22, 23, 24, 25, 26, 27 |
| 694 | 16, 17, 18, 19, 20, 21, 22, 23, 24, 25, 26, 27 |
| 695 | 16, 17, 18, 19, 20, 21, 22, 23, 24, 25, 26, 27 |
| 696 | 16, 17, 18, 19, 20, 21, 22, 23, 24, 25, 26, 27 |
| 697 | 16, 17, 18, 19, 20, 21, 22, 23, 24, 25, 26, 27 |
| 698 | 16, 17, 18, 19, 20, 21, 22, 23, 24, 25, 26, 27 |
| 699 | 16, 17, 18, 19, 20, 21, 22, 23, 24, 25, 26, 27 |
| 700 | 16, 17, 18, 19, 20, 21, 22, 23, 24, 25, 26, 27 |
| 701 | 16, 17, 18, 19, 20, 21, 22, 23, 24, 25, 26, 27 |
| 702 | 16, 17, 18, 19, 20, 21, 22, 23, 24, 25, 26, 27 |

Prototype Oligonucleotides:

| Sequence | Trivial | Starting | |
|---|---|---|---|
| ID No. | Name | Position* | 5′-->3′ Sequence |
| 61 | OL(11)SGP2 | 691 | CCGCAGGCAG CCCGTGGAGT TGT |
| 62 | OL(12)SGP2 | 700 | TCCTTCATCC GCAGGCAGCC CG |

*Because this is antisense, the position numbers must be read right to left; the starting and ending
positions are defined according to the GenBank sequence with the first base of the sense strand
being base number one.
Gene: SGP2
GenBank: HUMSGLY/M74816
References: Danik et al., Proc. Natl. Acad. Sci. 88; 8577 (1991).
HOT-SPOT 8. Range of bases included: positions 774-812*
Antisense Strand Sequence:
SEQ ID NO: 63: GGATTCGTCG AGCTCCCGCC GCAGCTTAGC CTGGGAGGG

| Nucleotide Starting | Size Variants |
|---|---|
| Position* | (Number of bases in the oligomer) |
| 774 | 16, 17, 18, 19, 20, 21, 22, 23, 24, 25 |
| 775 | 16, 17, 18, 19, 20, 21, 22, 23, 24 |
| 776 | 16, 17, 18, 19, 20, 21, 22, 23 |
| 777 | 16, 17, 18, 19, 20, 21, 22 |
| 778 | 16, 17, 18, 19, 20, 21, 22, 23, 24 |
| 779 | 16, 17, 18, 19, 20, 21, 22, 23 |
| 780 | 16, 17, 18, 19, 20, 21, 22 |
| 781 | 16, 17, 18, 19, 20, 21 |
| 782 | 16, 17, 18, 19, 20 |
| 783 | 16, 17, 18, 19 |
| 784 | 16, 17, 18 |
| 785 | 16, 17 |
| 786 | 16 |
| 787 | 16, 17, 18, 19, 20, 21, 22, 23, 24, 25 |
| 788 | 16, 17, 18, 19, 20, 21, 22, 23, 24 |
| 789 | 16, 17, 18, 19, 20, 21, 22, 23 |
| 790 | 16, 17, 18, 19, 20, 21, 22 |
| 791 | 16, 17, 18, 19, 20, 21 |
| 792 | 16, 17, 18, 19, 20 |
| 793 | 16, 17, 18, 19 |
| 794 | 16, 17, 18 |
| 795 | 16, 17 |
| 796 | 16 |
| 797 | 16, 17, 18, 19, 20, 21, 22, 23, 24, 25, 26, 27 |

Prototype Oligonucleotides:

| Sequence | Trivial | Starting | |
|---|---|---|---|
| ID No. | Name | Position* | 5′-->3′ Sequence |
| 64 | OL(13)SGP2 | 778 | TCCCGCCGCA GCTTAGCCTG GG |
| 65 | OL(14)SGP2 | 788 | TTCGTCGAGC TCCCGCCGCA GC |

*Because this is antisense, the position numbers must be read right to left; the starting and ending
positions are defined according to the GenBank sequence with the first base of the sense strand
being base number one.
Gene: SGP2
GenBank: HUMSGLY/M74816
References: Danik et al., Proc. Natl. Acad. Sci. 88; 8577 (1991).
HOT-SPOT 9. Range of bases included: positions 972-1002*
Antisense Strand Sequence:
SEQ ID NO: 66: AAGTGTGGGA AGCCACCGTG GTGACCCGCA G

| Nucleotide Starting | Size Variants |
|---|---|
| Position* | (Number of bases in the oligomer) |
| 972 | 16, 17, 18, 19, 20, 21, 22, 23, 24, 25, 26, 27 |
| 973 | 16, 17, 18, 19, 20, 21, 22, 23, 24, 25, 26, 27 |
| 974 | 16, 17, 18, 19, 20, 21, 22, 23, 24, 25, 26, 27 |
| 975 | 16, 17, 18, 19, 20, 21, 22, 23, 24, 25, 26, 27 |
| 976 | 16, 17, 18, 19, 20, 21, 22, 23, 24, 25, 26, 27 |

Prototype Oligonucleotides:

| Sequence | Trivial | Starting | |
|---|---|---|---|
| ID No. | Name | Position* | 5′-->3′ Sequence |
| 67 | OL(15)SGP2 | 974 | GGAAGCCACC GTGGTGACCC GC |

*Because this is antisense, the position numbers must be read right to left; the starting and ending
positions are defined according to the GenBank sequence with the first base of the sense strand
being base number one.
The Human TRPM-2 (exons 7, 8, 9) Gene
Gene: TRPM-2 (exons 7, 8, 9) GenBank: HUMTRPM2A4/M63379
References: Pineault et al., J. Biol. Chem. 268; 5021 (1993).
HOT-SPOT 1. Range of bases included: positions 1159-1198*
Antisense Strand Sequence:
SEQ ID NO: 68: CTTTAGCAGC TCGTTGTATT TCCTGGTCAA CCTCTCAGCG

| Nucleotide Starting | Size Variants |
|---|---|
| Position* | (Number of bases in the oligomer) |
| 1159 | 16, 17, 18, 19, 20, 21, 22, 23, 24, 25, 26, 27 |
| 1160 | 16, 17, 18, 19, 20, 21, 22, 23, 24, 25, 26, 27 |
| 1161 | 16, 17, 18, 19, 20, 21, 22, 23, 24, 25, 26, 27 |
| 1162 | 16, 17, 18, 19, 20, 21, 22, 23, 24, 25, 26, 27 |
| 1163 | 16, 17, 18, 19, 20, 21, 22, 23, 24, 25, 26, 27 |
| 1164 | 16, 17, 18, 19, 20, 21, 22, 23, 24, 25, 26, 27 |
| 1165 | 16, 17, 18, 19, 20, 21, 22, 23, 24, 25, 26, 27 |
| 1166 | 16, 17, 18, 19, 20, 21, 22, 23, 24, 25, 26, 27 |
| 1167 | 16, 17, 18, 19, 20, 21, 22, 23, 24, 25, 26, 27 |
| 1168 | 16, 17, 18, 19, 20, 21, 22, 23, 24, 25, 26, 27 |
| 1169 | 16, 17, 18, 19, 20, 21, 22, 23, 24, 25, 26, 27 |
| 1170 | 16, 17, 18, 19, 20, 21, 22, 23, 24, 25, 26, 27 |
| 1171 | 16, 17, 18, 19, 20, 21, 22, 23, 24, 25, 26, 27 |
| 1172 | 16, 17, 18, 19, 20, 21, 22, 23, 24, 25, 26, 27 |

Prototype Oligonucleotides:

| Sequence | Trivial | Starting | |
|---|---|---|---|
| ID No. | Name | Position* | 5′-->3′ Sequence |
| 69 | OL(31)TRPM-2 | 1162 | GGMGCCAG CCGGGACACC CA |
| 70 | OL(32)TRPM-2 | 1169 | TGCGTGAGGT TTGCCAGCCG GG |

*Because this is antisense, the position numbers must be read right to left; the starting and ending
positions are defined according to the GenBank sequence with the first base of the sense strand
being base number one.
Gene: TRPM-2 (exons 7, 8, 9)
GenBank: HUMTRPM2A4/M63379
References: Pineault et al., J. Biol. Chem. 268; 5021 (1993).
HOT-SPOT 2. Range of bases included: positions 3056-3093*
Antisense Strand Sequence: SEQ ID NO: 71: TACTTATM CCATCCCCCC TGCCTGCCCC
CCATAGCA

| Nucleotide Starting | Size Variants |
|---|---|
| Position* | (Number of bases in the oligomer) |
| 3056 | 16, 17, 18, 19, 20, 21, 22, 23, 24, 25, 26, 27 |
| 3057 | 16, 17, 18, 19, 20, 21, 22, 23, 24, 25, 26, 27 |
| 3058 | 16, 17, 18, 19, 20, 21, 22, 23, 24, 25, 26, 27 |
| 3059 | 16, 17, 18, 19, 20, 21, 22, 23, 24, 25, 26, 27 |
| 3060 | 16, 17, 18, 19, 20, 21, 22, 23, 24, 25, 26, 27 |
| 3061 | 16, 17, 18, 19, 20, 21, 22, 23, 24, 25, 26, 27 |
| 3062 | 16, 17, 18, 19, 20, 21, 22, 23, 24, 25, 26, 27 |
| 3063 | 16, 17, 18, 19, 20, 21, 22, 23, 24, 25, 26, 27 |
| 3064 | 16, 17, 18, 19, 20, 21, 22, 23, 24, 25, 26, 27 |
| 3065 | 16, 17, 18, 19, 20, 21, 22, 23, 24, 25, 26, 27 |
| 3066 | 16, 17, 18, 19, 20, 21, 22, 23, 24, 25, 26, 27 |
| 3067 | 16, 17, 18, 19, 20, 21, 22, 23, 24, 25, 26, 27 |

Prototype Oligonucleotides:

| Sequence | Trivial | Starting | |
|---|---|---|---|
| ID No. | Name | Position* | 5′-->3′ Sequence |
| 72 | OL(33)TRPM-2 | 3061 | TTCCATCCCC CCTGCCTGCC CCCCA |

*Because this is antisense, the position numbers must be read right to left; the starting and ending
positions are defined according to the GenBank sequence with the first base of the sense strand
being base number one.
Gene: TRPM-2 (exong 7, 8, 9)
GenBank: HUMTRPM2A4/M63379
References: Pineault et al., J. Biol. Chem. 268; 5021 (1993).
HOT-SPOT 3. Range of bases included: positions 1012-1050*
Antisense Strand Sequence:
SEQ ID NO: 73: GGATTCGTCG AGCTCCCGCC GCAGCTTAGC CTGGGAGGG

| Nucleotide Starting | Size Variants |
|---|---|
| Position* | (Number of bases in the oligomer) |
| 1012 | 16, 17, 18, 19, 20, 21, 22, 23, 24, 25 |
| 1013 | 16, 17, 18, 19, 20, 21, 22, 23, 24 |
| 1014 | 16, 17, 18, 19, 20, 21, 22, 23 |
| 1015 | 16, 17, 18, 19, 20, 21, 22 |
| 1016 | 16, 17, 18, 19, 20, 21, 22, 23, 24 |
| 1017 | 16, 17, 18, 19, 20, 21, 22, 23 |
| 1018 | 16, 17, 18, 19, 20, 21, 22 |
| 1019 | 16, 17, 18, 19, 20, 21 |
| 1020 | 16, 17, 18, 19, 20 |
| 1021 | 16, 17, 18, 19 |
| 1022 | 16, 17, 18 |
| 1023 | 16, 17 |
| 1024 | 16 |
| 1025 | |
| 1026 | 16, 17, 18, 19, 20, 21, 22, 23, 24, 25 |
| 1027 | 16, 17, 18, 19, 20, 21, 22, 23, 24 |
| 1028 | 16, 17, 18, 19, 20, 21, 22, 23 |
| 1029 | 16, 17, 18, 19, 20, 21, 22 |
| 1030 | 16, 17, 18, 19, 20, 21 |
| 1031 | 16, 17, 18, 19, 20 |
| 1032 | 16, 17, 18, 19 |
| 1033 | 16, 17, 18 |

Prototype Oligonucleotides:

| Sequence | Trivial | Starting | |
|---|---|---|---|
| ID No. | Name | Position* | 5′-->3′ Sequence |
| 74 | OL(34)TRPM-2 | 1016 | TCCCGCCGCA GCTTAGCCTG GG |
| 75 | OL(35)TRPM-2 | 1026 | TTCGTCGAGC TCCCGCCGCA GC |

*Because this is antisense, the position numbers must be read right to left; the starting and ending
positions are defined according to the GenBank sequence with the first base of the sense strand
being base number one.
Gene: TRPM-2 (exons 7, 8, 9) PCT/US2012/046463
GenBank: HUMTRPM2A4/M63379
References: Pineault et al., J. Biol. Chem. 268; 5021 (1993).
HOT-SPOT 4. Range of bases included: positions 2623-2669*
Antisense Strand Sequence:
SEQ ID NO: 76: GCTTTGTTCT TGGGCTCTGG CTCCGGGCGG AGTCGTTCCC
ACCAGGC

| Nucleotide Starting | Size Variants |
|---|---|
| Position* | (Number of bases in the oligomer) |
| 2623 | 16, 17, 18, 19, 20, 21, 22 |
| 2624 | 16, 17, 18, 19, 20, 21 |
| 2625 | 16, 17, 18, 19, 20 |
| 2626 | 16, 17, 18, 19 |
| 2627 | 16, 17, 18 |
| 2628 | 16, 17 |
| 2629 | 16 |
| 2630 | |
| 2631 | 16, 17, 18, 19, 20, 21, 22, 23, 24, 25, 26, 27 |
| 2632 | 16, 17, 18, 19, 20, 21, 22, 23, 24, 25, 26, 27 |
| 2633 | 16, 17, 18, 19, 20, 21, 22, 23, 24, 25, 26, 27 |
| 2634 | 16, 17, 18, 19, 20, 21, 22, 23, 24, 25, 26, 27 |
| 2635 | 16, 17, 18, 19, 20, 21, 22, 23, 24, 25, 26, 27 |
| 2636 | 16, 17, 18, 19, 20, 21, 22, 23, 24, 25, 26, 27 |
| 2637 | 16, 17, 18, 19, 20, 21, 22, 23, 24, 25, 26, 27 |
| 2638 | 16, 17, 18, 19, 20, 21, 22, 23, 24, 25, 26, 27 |
| 2639 | 16, 17, 18, 19, 20, 21, 22, 23, 24, 25, 26, 27 |
| 2640 | 16, 17, 18, 19, 20, 21, 22, 23, 24, 25, 26, 27 |
| 2641 | 16, 17, 18, 19, 20, 21, 22, 23, 24, 25, 26, 27 |
| 2642 | 16, 17, 18, 19, 20, 21, 22, 23, 24, 25, 26, 27 |
| 2643 | 16, 17, 18, 19, 20, 21, 22, 23, 24, 25, 26, 27 |

Prototype Oligonucleotides:

| Sequence | Trivial | Starting | |
|---|---|---|---|
| ID No. | Name | Position* | 5′-->3′ Sequence |
| 77 | OL(36)TRPM-2 | 2623 | GGCGGAGTCG TTCCCACCAG GC |
| 78 | OL(37)TRPM-2 | 2639 | TTGGGCTCTG GCTCCGGGCG GA |

*Because this is antisense, the position numbers must be read right to left; the starting and ending
positions are defined according to the GenBank sequence with the first base of the sense
strand being base number one.
Gene: TRPM-2 (exons 7, 8, 9)
GenBank: HUMTRPM2A4/M63379
References: Pineault et al., J. Biol. Chem. 268; 5021 (1993).
HOT-SPOT 5. Range of bases included: positions 2876-2898*
Antisense Strand Sequence:
SEQ ID NO: 79: GGGAGAGGCT GGGCGGAGTT GGG

| Nucleotide Starting | Size Variants |
|---|---|
| Position* | (Number of bases in the oligomer) |
| 2876 | 16, 17, 18, 19, 20, 21, 22, 23 |
| 2877 | 16, 17, 18, 19, 20, 21, 22 |
| 2878 | 16, 17, 18, 19, 20, 21 |
| 2879 | 16, 17, 18, 19, 20 |
| 2880 | 16, 17, 18, 19 |
| 2881 | 16, 17, 18 |
| 2882 | 16, 17 |
| 2883 | 16 |

Prototype Oligonucleotides:

| Sequence | Trivial | Starting | |
|---|---|---|---|
| ID No. | Name | Position* | 5′-->3′ Sequence |
| 80 | OL(38)TRPM-2 | 2876 | GGGAGAGGCT GGGCGGAGTT GGG |

*Because this is antisense, the position numbers must be read right to left; the starting and ending
positions are defined according to the GenBank sequence with the first base of the sense strand
being base number one.
The Human TRPM-2 (exons 1, 2, 3) Gene
Gene: TRPM-2 (exons 1, 2, 3)
GenBank: HUMTRPM2A1/M63376
References: Pineault et al., J. Biol. Chem. 268; 5021 (1993).
HOT-SPOT 1. Range of bases included: positions 1-27*
Antisense Strand Sequence:
SEQ ID NO: 81: GGGAATGGAT CCGTTGACCT GCAGGTC

| Nucleotide Starting | Size Variants |
|---|---|
| Position* | (Number of bases in the oligomer) |
| 1 | 16, 17, 18, 19, 20, 21, 22, 23, 24, 25, 26, 27 |
| 2 | 16, 17, 18, 19, 20, 21, 22, 23, 24, 25, 26 |
| 3 | 16, 17, 18, 19, 20, 21, 22, 23, 24, 25 |
| 4 | 16, 17, 18, 19, 20, 21, 22, 23, 24 |
| 5 | 16, 17, 18, 19, 20, 21, 22, 23 |
| 6 | 16, 17, 18, 19, 20, 21, 22 |
| 7 | 16, 17, 18, 19, 20, 21 |
| 8 | 16, 17, 18, 19, 20 |
| 9 | 16, 17, 18, 19 |
| 10 | 16, 17, 18 |
| 11 | 16, 17 |
| 12 | 16 |

Prototype Oligonucleotides:

| Sequence | Trivial | Starting | |
|---|---|---|---|
| ID No. | Name | Position* | 5′-->3′ Sequence |
| 82 | OL(1)TRPM2 | 6 | GGGAATGGAT CCGTTGACCT GC |

*Because this is antisense, the position numbers must be read right to left; the starting and ending
positions are defined according to the GenBank sequence with the first base of the sense strand
being base number one.
Gene: TRPM-2 (exons 1, 2, 3)
GenBank: HUMTRPM2A1/M63376
References: Pineault et al., J. Biol. Chem. 268; 5021 (1993).
HOT-SPOT 2. Range of bases included: positions 381-402*
Antisense Strand Sequence:
SEQ ID NO: 83: GGGCAATGGG TGGACAGGGC GG

| Nucleotide Starting | Size Variants |
|---|---|
| Position* | (Number of bases in the oligomer) |
| 381 | 16, 17, 18, 19, 20, 21, 22 |
| 382 | 16, 17, 18, 19, 20, 21 |
| 383 | 16, 17, 18, 19, 20 |
| 384 | 16, 17, 18, 19 |
| 385 | 16, 17, 18 |
| 386 | 16, 17 |
| 387 | 16 |

Prototype Oligonucleotides:

| Sequence | Trivial | Starting | |
|---|---|---|---|
| ID No. | Name | Position* | 5′-->3′ Sequence |
| 84 | OL(2)TRPM2 | 382 | GGGCAATGGG TGGACAGGGC G |

*Because this is antisense, the position numbers must be read right to left; the starting and ending
positions are defined according to the GenBank sequence with the first base of the sense strand
being base number one.
Gene: TRPM-2 (exons 1, 2, 3)
GenBank: HUMTRPM2A1/M63376
References: Pineault et al., J. Biol. Chem. 268; 5021 (1993).
HOT-SPOT 3. Range of bases included: positions 525-561*
Antisense Strand Sequence:
SEQ ID NO: 85: AGGGAGAATC TCCTGTCGCA GCCACCCCTC CCCCTGG

| Nucleotide Starting | Size Variants |
|---|---|
| Position* | (Number of bases in the oligomer) |
| 525 | 16, 17, 18, 19, 20, 21, 22, 23, 24, 25, 26, 27 |
| 526 | 16, 17, 18, 19, 20, 21, 22, 23, 24, 25, 26, 27 |
| 527 | 16, 17, 18, 19, 20, 21, 22, 23, 24, 25, 26, 27 |
| 528 | 16, 17, 18, 19, 20, 21, 22, 23, 24, 25, 26, 27 |
| 529 | 16, 17, 18, 19, 20, 21, 22, 23, 24, 25, 26, 27 |
| 530 | 16, 17, 18, 19, 20, 21, 22, 23, 24, 25, 26, 27 |
| 531 | 16, 17, 18, 19, 20, 21, 22, 23, 24, 25, 26, 27 |
| 532 | 16, 17, 18, 19, 20, 21, 22, 23, 24, 25, 26, 27 |
| 533 | 16, 17, 18, 19, 20, 21, 22, 23, 24, 25, 26, 27 |
| 534 | 16, 17, 18, 19, 20, 21, 22, 23, 24, 25, 26, 27 |
| 535 | 16, 17, 18, 19, 20, 21, 22, 23, 24, 25, 26, 27 |

Prototype Oligonucleotides:

| Sequence | Trivial | Starting | |
|---|---|---|---|
| ID No. | Name | Position* | 5′-->3′ Sequence |
| 86 | OL(3)TRPM2 | 525 | TCGCAGCCAC CCCTCCCCCT GG |

*Because this is antisense, the position numbers must be read right to left; the starting and ending
positions are defined according to the GenBank sequence with the first base of the sense strand
being base number one.
Gene: TRPM-2 (exons 1, 2, 3)
GenBank: HUMTRPM2A1/M63376
References: Pineault et al., J. Biol. Chem. 268; 5021 (1993).
HOT-SPOT 4. Range of bases included: positions 1511-1542*
Antisense Strand Sequence:
SEQ ID NO: 87: GCTGCCATCC CCTGCCCGCC CATCCGTCCT GG

| Nucleotide Starting | Size Variants |
|---|---|
| Position* | (Number of bases in the oligomer) |
| 1511 | 16, 17, 18, 19, 20, 21, 22, 23, 24, 25, 26, 27 |
| 1512 | 16, 17, 18, 19, 20, 21, 22, 23, 24, 25, 26, 27 |
| 1513 | 16, 17, 18, 19, 20, 21, 22, 23, 24, 25, 26, 27 |
| 1514 | 16, 17, 18, 19, 20, 21, 22, 23, 24, 25, 26, 27 |
| 1515 | 16, 17, 18, 19, 20, 21, 22, 23, 24, 25, 26, 27 |
| 1516 | 16, 17, 18, 19, 20, 21, 22, 23, 24, 25, 26, 27 |
| 1517 | 16, 17, 18, 19, 20, 21, 22, 23, 24, 25, 26 |
| 1518 | 16, 17, 18, 19, 20, 21, 22, 23, 24, 25 |
| 1519 | 16, 17, 18, 19, 20, 21, 22, 23, 24 |
| 1520 | 16, 17, 18, 19, 20, 21, 22, 23 |
| 1521 | 16, 17, 18, 19, 20, 21, 22 |
| 1522 | 16, 17, 18, 19, 20, 21 |
| 1523 | 16, 17, 18, 19, 20 |
| 1524 | 16, 17, 18, 19 |
| 1525 | 16, 17, 18 |
| 1526 | 16, 17 |
| 1527 | 16 |

Prototype Oligonucleotides:

| Sequence | Trivial | Starting | |
|---|---|---|---|
| ID No. | Name | Position* | 5′-->3′ Sequence |
| 88 | OL(4)TRPM2 | 1514 | TCCCCTGCCC GCCCATCCGT CC |

*Because this is antisense, the position numbers must be read right to left; the starting and ending
positions are defined according to the GenBank sequence with the first base of the sense strand
being base number one.
Gene: TRPM-2 (exons 1, 2, 3)
GenBank: HUMTRPM2A1/M63376
References: Pineault et al., J. Biol. Chem. 268; 5021 (1993).
HOT-SPOT 5. Range of bases included: positions 1587-1628*
Antisense Strand Sequence:
SEQ ID NO: 89: CTCCGGCACC TCCCCTCCCT GGCTTCCTGG GCTCCGTCGA AA

| Nucleotide Starting | Size Variants |
|---|---|
| Position* | (Number of bases in the oligomer) |
| 1587 | 16, 17, 18, 19, 20, 21, 22, 23, 24, 25 |
| 1588 | 16, 17, 18, 19, 20, 21, 22, 23, 24 |
| 1589 | 16, 17, 18, 19, 20, 21, 22, 23 |
| 1590 | 16, 17, 18, 19, 20, 21, 22 |
| 1591 | 16, 17, 18, 19, 20, 21 |
| 1592 | 16, 17, 18, 19, 20 |
| 1593 | 16, 17, 18, 19 |
| 1594 | 16, 17, 18 |
| 1595 | 16, 17 |
| 1596 | 16 |
| 1597 | |
| 1598 | |
| 1599 | 16, 17, 18, 19, 20, 21, 22, 23, 24, 25, 26, 27 |
| 1600 | 16, 17, 18, 19, 20, 21, 22, 23, 24, 25, 26, 27 |
| 1601 | 16, 17, 18, 19, 20, 21, 22, 23, 24, 25, 26, 27 |
| 1602 | 16, 17, 18, 19, 20, 21, 22, 23, 24, 25, 26, 27 |
| 1603 | 16, 17, 18, 19, 20, 21, 22, 23, 24, 25, 26, 27 |
| 1604 | 16, 17, 18, 19, 20, 21, 22, 23, 24, 25, 26, 27 |
| 1605 | 16, 17, 18, 19, 20, 21, 22, 23, 24, 25, 26 |
| 1606 | 16, 17, 18, 19, 20, 21, 22, 23, 24, 25 |
| 1607 | 16, 17, 18, 19, 20, 21, 22, 23, 24 |
| 1608 | 16, 17, 18, 19, 20, 21, 22, 23 |
| 1609 | 16, 17, 18, 19, 20, 21, 22 |
| 1610 | 16, 17, 18, 19, 20, 21 |
| 1611 | 16, 17, 18, 19, 20 |
| 1612 | 16, 17, 18, 19 |
| 1613 | 16, 17, 18 |

Prototype Oligonucleotides:

| Sequence | Trivial | Starting | |
|---|---|---|---|
| ID No. | Name | Position* | 5′-->3′ Sequence |
| 90 | OL(5)TRPM2 | 1590 | CCTGGCTTCC TGGGCTCCGT CG |
| 91 | OL(6)TRPM2 | 1606 | TCCGGCACCT CCCCTCCCTG GC |

*Because this is antisense, the position numbers must be read right to left; the starting and ending
positions are defined according to the GenBank sequence with the first base of the sense strand
being base number one.
Gene: TRPM-2 (exons 1, 2, 3)
GenBank: HUMTRPM2A1/M63376
References: Pineault et al., J. Biol. Chem. 268; 5021 (1993).
HOT-SPOT 6. Range of bases included: positions 1660-1696*
Antisense Strand Sequence:
SEQ ID NO: 92: GTTGCGTGAA GCTCGTGCTC ATCAGGCGGC GGTTGCG

| Nucleotide Starting | Size Variants |
|---|---|
| Position* | (Number of bases in the oligomer) |
| 1660 | 16, 17, 18, 19, 20, 21, 22, 23, 24, 25, 26, 27 |
| 1661 | 16, 17, 18, 19, 20, 21, 22, 23, 24, 25, 26, 27 |
| 1662 | 16, 17, 18, 19, 20, 21, 22, 23, 24, 25, 26, 27 |
| 1663 | 16, 17, 18, 19, 20, 21, 22, 23, 24, 25, 26, 27 |
| 1664 | 16, 17, 18, 19, 20, 21, 22, 23, 24, 25, 26, 27 |
| 1665 | 16, 17, 18, 19, 20, 21, 22, 23, 24, 25, 26, 27 |
| 1666 | 16, 17, 18, 19, 20, 21, 22, 23, 24, 25, 26, 27 |
| 1667 | 16, 17, 18, 19, 20, 21, 22, 23, 24, 25, 26, 27 |
| 1668 | 16, 17, 18, 19, 20, 21, 22, 23, 24, 25, 26, 27 |
| 1669 | 16, 17, 18, 19, 20, 21, 22, 23, 24, 25, 26, 27 |
| 1670 | 16, 17, 18, 19, 20, 21, 22, 23, 24, 25, 26, 27 |

Prototype Oligonucleotides:

| Sequence | Trivial | Starting | |
|---|---|---|---|
| ID No. | Name | Position* | 5′-->3′ Sequence |
| 93 | OL(7)TRPM2 | 1660 | TGCTCATCAG GCGGCGGTTG CG |

*Because this is antisense, the position numbers must be read right to left; the starting and ending
positions are defined according to the GenBank sequence with the first base of the sense strand
being base number one.
Gene: TRPM-2 (exons 1, 2, 3)
GenBank: HUMTRPM2A1/M63376
References: Pineault et al., J. Biol. Chem. 268; 5021 (1993).
HOT-SPOT 7. Range of bases included: positions 3834-3868*
Antisense Strand Sequence:
SEQ ID NO: 94: GGATGAGAGC CCAGCCCTCC CAGCCCCATG TCCAG

| Nucleotide Starting | Size Variants |
|---|---|
| Position* | (Number of bases in the oligomer) |
| 3834 | 16, 17, 18, 19, 20, 21, 22, 23, 24, 25, 26, 27 |
| 3835 | 16, 17, 18, 19, 20, 21, 22, 23, 24, 25, 26, 27 |
| 3836 | 16, 17, 18, 19, 20, 21, 22, 23, 24, 25, 26, 27 |
| 3837 | 16, 17, 18, 19, 20, 21, 22, 23, 24, 25, 26, 27 |
| 3838 | 16, 17, 18, 19, 20, 21, 22, 23, 24, 25, 26, 27 |
| 3839 | 16, 17, 18, 19, 20, 21, 22, 23, 24, 25, 26, 27 |
| 3840 | 16, 17, 18, 19, 20, 21, 22, 23, 24, 25, 26, 27 |
| 3841 | 16, 17, 18, 19, 20, 21, 22, 23, 24, 25, 26, 27 |
| 3842 | 16, 17, 18, 19, 20, 21, 22, 23, 24, 25, 26, 27 |
| 3843 | 16, 17, 18, 19, 20, 21, 22, 23, 24, 25, 26 |
| 3844 | 16, 17, 18, 19, 20, 21, 22, 23, 24, 25 |
| 3845 | 16, 17, 18, 19, 20, 21, 22, 23, 24 |
| 3846 | 16, 17, 18, 19, 20, 21, 22, 23 |
| 3847 | 16, 17, 18, 19, 20, 21, 22 |
| 3848 | 16, 17, 18, 19, 20, 21 |
| 3849 | 16, 17, 18, 19, 20 |
| 3850 | 16, 17, 18, 19 |
| 3851 | 16, 17, 18 |
| 3852 | 16, 17 |
| 3853 | 16 |

Prototype Oligonucleotides:

| Sequence | Trivial | Starting | |
|---|---|---|---|
| ID No. | Name | Position* | 5′-->3′ Sequence |
| 95 | OL(8)TRPM2 | 3839 | GCCCAGCCCT CCCAGCCCCA TG |

*Because this is antisense, the position numbers must be read right to left; the starting and ending
positions are defined according to the GenBank sequence with the first base of the sense strand
being base number one.
Gene: TRPM-2 (exons 1, 2, 3)
GenBank: HUMTRPM2A1/M63376
References: Pineault et al., J. Biol. Chem. 268; 5021 (1993).
HOT-SPOT 8. Range of bases included: positions 47154737*
Antisense Strand Sequence:
SEQ ID NO: 96: GCCCTCCACA CTGCCCATCC CCT

| Nucleotide Starting | Size Variants |
|---|---|
| Position* | (Number of bases in the oligomer) |
| 4715 | 16, 17, 18, 19, 20, 21, 22, 23 |
| 4716 | 16, 17, 18, 19, 20, 21, 22 |
| 4717 | 16, 17, 18, 19, 20, 21 |
| 4718 | 16, 17, 18, 19, 20 |
| 4719 | 16, 17, 18, 19 |
| 4720 | 16, 17, 18 |
| 4721 | 16, 17 |
| 4722 | 16 |

Prototype Oligonucleotides:

| Sequence | Trivial | Starting | |
|---|---|---|---|
| ID No. | Name | Position* | 5′-->3′ Sequence |
| 97 | OL(9)TRPM2 | 4715 | GCCCTCCACA CTGCCCATCC CCT |

*Because this is antisense, the position numbers must be read right to left; the starting and ending
positions are defined according to the GenBank sequence with the first base of the sense strand
being base number one.
Gene: TRPM-2 (exons 1, 2, 3)
GenBank: HUMTRPM2A1/M63376
References: Pineault et al., J. Biol. Chem. 268; 5021 (1993).
HOT-SPOT 9. Range of bases included: positions 5434-5471*
Antisense Strand Sequence:
SEQ ID NO: 98: CAGCCTGCTN NGTCCCGCCC ACTCCGCCCT GCAGCAGC

| Nucleotide Starting | Size Variants |
|---|---|
| Position* | (Number of bases in the oligomer) |
| 5434 | 16, 17, 18, 19, 20, 21, 22, 23, 24, 25, 26, 27 |
| 5435 | 16, 17, 18, 19, 20, 21, 22, 23, 24, 25, 26, 27 |
| 5436 | 16, 17, 18, 19, 20, 21, 22, 23, 24, 25, 26, 27 |
| 5437 | 16, 17, 18, 19, 20, 21, 22, 23, 24, 25, 26, 27 |
| 5438 | 16, 17, 18, 19, 20, 21, 22, 23, 24, 25, 26, 27 |
| 5439 | 16, 17, 18, 19, 20, 21, 22, 23, 24, 25, 26, 27 |
| 5440 | 16, 17, 18, 19, 20, 21, 22, 23, 24, 25, 26, 27 |
| 5441 | 16, 17, 18, 19, 20, 21, 22, 23, 24, 25, 26, 27 |
| 5442 | 16, 17, 18, 19, 20, 21, 22, 23, 24, 25, 26, 27 |
| 5443 | 16, 17, 18, 19, 20, 21, 22, 23, 24, 25, 26, 27 |
| 5444 | 16, 17, 18, 19, 20, 21, 22, 23, 24, 25, 26, 27 |
| 5445 | 16, 17, 18, 19, 20, 21, 22, 23, 24, 25, 26, 27 |

Prototype Oligonucleotides:

| Sequence | Trivial | Starting | |
|---|---|---|---|
| ID No. | Name | Position* | 5′-->3′ Sequence |
| 99 | OL(10)TRPM2 | 5436 | CCGCCCACTC CGCCCTGCAG CA |

*Because this is antisense, the position numbers must be read right to left; the starting and ending
positions are defined according to the GenBank sequence with the first base of the sense strand
being base number one.
Gene: TRPM-2 (exons 1, 2, 3)
GenBank: HUMTRPM2A1/M63376
References: Pineault et al., J. Biol. Chem. 268; 5021 (1993).
HOT-SPOT 10. Range of bases included: positions 5562-5594*
Antisense Strand Sequence:
SEQ ID NO: 100: CTGCCCACTC TCCCAGGTCA GCAGCAGCCC CAC

| Nucleotide Starting | Size Variants |
|---|---|
| Position* | (Number of bases in the oligomer) |
| 5562 | 16, 17, 18, 19, 20, 21, 22, 23, 24, 25, 26, 27 |
| 5563 | 16, 17, 18, 19, 20, 21, 22, 23, 24, 25, 26, 27 |
| 5564 | 16, 17, 18, 19, 20, 21, 22, 23, 24, 25, 26, 27 |
| 5565 | 16, 17, 18, 19, 20, 21, 22, 23, 24, 25, 26, 27 |
| 5566 | 16, 17, 18, 19, 20, 21, 22, 23, 24, 25, 26, 27 |
| 5567 | 16, 17, 18, 19, 20, 21, 22, 23, 24, 25, 26, 27 |
| 5568 | 16, 17, 18, 19, 20, 21, 22, 23, 24, 25, 26, 27 |

Prototype Oligonucleotides:

| Sequence | Trivial | Starting | |
|---|---|---|---|
| ID No. | Name | Position* | 5′-->3′ Sequence |
| 101 | OL(11)TRPM2 | 5563 | TCCCAGGTCA GCAGCAGCCC CA |

*Because this is antisense, the position numbers must be read right to left; the starting and ending
positions are defined according to the GenBank sequence with the first base of the sense strand
being base number one.
Gene: TRPM-2 (exons 1, 2, 3)
GenBank: HUMTRPM2A1/M63376
References: Pineault et al., J. Biol. Chem. 268; 5021 (1993).
HOT-SPOT 11. Range of bases included: positions 6407-6449*
Antisense Strand Sequence:
SEQ ID NO: 102: GTTTACGTAA TGCTCCAGCC CCCCGCCGTG CCCACTTCGC AGA

| Nucleotide Starting | Size Variants |
|---|---|
| Position* | (Number of bases in the oligomer) |
| 6407 | 16, 17, 18, 19, 20, 21, 22, 23, 24, 25, 26, 27 |
| 6408 | 16, 17, 18, 19, 20, 21, 22, 23, 24, 25, 26, 27 |
| 6409 | 16, 17, 18, 19, 20, 21, 22, 23, 24, 25, 26, 27 |
| 6410 | 16, 17, 18, 19, 20, 21, 22, 23, 24, 25, 26, 27 |
| 6411 | 16, 17, 18, 19, 20, 21, 22, 23, 24, 25, 26, 27 |
| 6412 | 16, 17, 18, 19, 20, 21, 22, 23, 24, 25, 26, 27 |
| 6413 | 16, 17, 18, 19, 20, 21, 22, 23, 24, 25, 26, 27 |
| 6414 | 16, 17, 18, 19, 20, 21, 22, 23, 24, 25, 26, 27 |
| 6415 | 16, 17, 18, 19, 20, 21, 22, 23, 24, 25, 26, 27 |
| 6416 | 16, 17, 18, 19, 20, 21, 22, 23, 24, 25, 26, 27 |
| 6417 | 16, 17, 18, 19, 20, 21, 22, 23, 24, 25, 26, 27 |
| 6418 | 16, 17, 18, 19, 20, 21, 22, 23, 24, 25, 26, 27 |
| 6419 | 16, 17, 18, 19, 20, 21, 22, 23, 24, 25, 26, 27 |
| 6420 | 16, 17, 18, 19, 20, 21, 22, 23, 24, 25, 26, 27 |
| 6421 | 16, 17, 18, 19, 20, 21, 22, 23, 24, 25, 26, 27 |
| 6422 | 16, 17, 18, 19, 20, 21, 22, 23, 24, 25, 26, 27 |
| 6423 | 16, 17, 18, 19, 20, 21, 22, 23, 24, 25, 26, 27 |

Prototype Oligonucleotides:

| Sequence | Trivial | Starting | |
|---|---|---|---|
| ID No. | Name | Position* | 5′-->3′ Sequence |
| 103 | OL(12)TRPM2 | 6410 | GCCCCCCGCC GTGCCCACTT CGC |
| 104 | OL(13)TRPM2 | 6418 | TGCTCCAGCC CCCCGCCGTG CC |

*Because this is antisense, the position numbers must be read right to left; the starting and ending
positions are defined according to the GenBank sequence with the first base of the sense strand
being base number one.
Gene: TRPM-2 (exons 1, 2, 3)
GenBank: HUMTRPM2A1/M63376
References: Pineault et al., J. Biol. Chem. 268; 5021 (1993).
HOT-SPOT 12. Range of bases included: positions 7178-7218*
Antisense Strand Sequence:
SEQ ID NO: 105: CCTCCAGTGG GATGGTCAAG GCAGGGAGGC GGTAGCGGCT C

| Nucleotide Starting | Size Variants |
|---|---|
| Position* | (Number of bases in the oligomer) |
| 7178 | 16, 17, 18, 19, 20, 21, 22, 23, 24, 25, 26, 27 |
| 7179 | 16, 17, 18, 19, 20, 21, 22, 23, 24, 25, 26, 27 |
| 7180 | 16, 17, 18, 19, 20, 21, 22, 23, 24, 25, 26, 27 |
| 7181 | 16, 17, 18, 19, 20, 21, 22, 23, 24, 25, 26, 27 |
| 7182 | 16, 17, 18, 19, 20, 21, 22, 23, 24, 25, 26, 27 |
| 7183 | 16, 17, 18, 19, 20, 21, 22, 23, 24, 25, 26, 27 |
| 7184 | 16, 17, 18, 19, 20, 21, 22, 23, 24, 25, 26, 27 |
| 7185 | 16, 17, 18, 19, 20, 21, 22, 23, 24, 25, 26, 27 |
| 7186 | 16, 17, 18, 19, 20, 21, 22, 23, 24, 25, 26, 27 |
| 7187 | 16, 17, 18, 19, 20, 21, 22, 23, 24, 25, 26, 27 |
| 7188 | 16, 17, 18, 19, 20, 21, 22, 23, 24, 25, 26, 27 |
| 7189 | 16, 17, 18, 19, 20, 21, 22, 23, 24, 25, 26, 27 |
| 7190 | 16, 17, 18, 19, 20, 21, 22, 23, 24, 25, 26, 27 |
| 7191 | 16, 17, 18, 19, 20, 21, 22, 23, 24, 25, 26, 27 |
| 7192 | 16, 17, 18, 19, 20, 21, 22, 23, 24, 25, 26, 27 |

Prototype Oligonucleotides:

| Sequence | Trivial | Starting | |
|---|---|---|---|
| ID No. | Name | Position* | 5′-->3′ Sequence |
| 106 | OL(14)TRPM2 | 7178 | GGCAGGGAGG CGGTAGCGGC TC |
| 107 | OL(15)TRPM2 | 7188 | GGGATGGTCA AGGCAGGGAG GCG |

*Because this is antisense, the position numbers must be read right to left; the starting and ending
positions are defined according to the GenBank sequence with the first base of the sense strand
being base number one.
Gene: TRPM-2 (exons 1, 2, 3)
GenBank: HUMTRPM2A1/M63376
References: Pineault et al., J. Biol. Chem. 268; 5021 (1993).
HOT-SPOT 13. Range of bases included: positions 7209-7245*
Antisense Strand Sequence:
SEQ ID NO: 108: GGCACCGCGC AGTGACCCCC TCCCTCCCCT CCAGTGG

| Nucleotide Starting | Size Variants |
|---|---|
| Position* | (Number of bases in the oligomer) |
| 7209 | 16, 17, 18, 19, 20, 21, 22, 23, 24, 25, 26, 27 |
| 7210 | 16, 17, 18, 19, 20, 21, 22, 23, 24, 25, 26, 27 |
| 7211 | 16, 17, 18, 19, 20, 21, 22, 23, 24, 25, 26, 27 |
| 7212 | 16, 17, 18, 19, 20, 21, 22, 23, 24, 25, 26, 27 |
| 7213 | 16, 17, 18, 19, 20, 21, 22, 23, 24, 25, 26, 27 |
| 7214 | 16, 17, 18, 19, 20, 21, 22, 23, 24, 25, 26, 27 |
| 7215 | 16, 17, 18, 19, 20, 21, 22, 23, 24, 25, 26, 27 |
| 7216 | 16, 17, 18, 19, 20, 21, 22, 23, 24, 25, 26, 27 |
| 7217 | 16, 17, 18, 19, 20, 21, 22, 23, 24, 25, 26, 27 |
| 7218 | 16, 17, 18, 19, 20, 21, 22, 23, 24, 25, 26, 27 |
| 7219 | 16, 17, 18, 19, 20, 21, 22, 23, 24, 25, 26, 27 |
| 7220 | 16, 17, 18, 19, 20, 21, 22, 23, 24, 25, 26 |
| 7221 | 16, 17, 18, 19, 20, 21, 22, 23, 24, 25 |
| 7222 | 16, 17, 18, 19, 20, 21, 22, 23, 24 |
| 7223 | 16, 17, 18, 19, 20, 21, 22, 23 |
| 7224 | 16, 17, 18, 19, 20, 21, 22 |
| 7225 | 16, 17, 18, 19, 20, 21 |
| 7226 | 16, 17, 18, 19, 20 |
| 7227 | 16, 17, 18, 19 |
| 7228 | 16, 17, 18 |
| 7229 | 16, 17 |
| 7230 | 16 |

Prototype Oligonucleotides:

| Sequence | Trivial | Starting | |
|---|---|---|---|
| ID No. | Name | Position* | 5′-->3′ Sequence |
| 109 | OL(16)TRPM2 | 7209 | CCCCCTCCCT CCCCTCCAGT GG |
| 110 | OL(17)TRPM2 | 7224 | GGCACCGCGC AGTGACCCCC TC |

*Because this is antisense, the position numbers must be read right to left; the starting and ending
positions are defined according to the GenBank sequence with the first base of the sense strand
being base number one.
Gene: TRPM-2 (exons 1, 2, 3)
GenBank: HUMTRPM2A1/M63376
References: Pineault et al., J. Biol. Chem. 268; 5021 (1993).
HOT-SPOT 14. Range of bases included: positions 7453-7482*
Antisense Strand Sequence:
SEQ ID NO: 111: GCCCCTGCCG CCACCCCACC CAGAGCCTGC

| Nucleotide Starting | Size Variants |
|---|---|
| Position* | (Number of bases in the oligomer) |
| 7453 | 16, 17, 18, 19, 20, 21, 22, 23, 24, 25, 26, 27 |
| 7454 | 16, 17, 18, 19, 20, 21, 22, 23, 24, 25, 26, 27 |
| 7455 | 16, 17, 18, 19, 20, 21, 22, 23, 24, 25, 26, 27 |
| 7456 | 16, 17, 18, 19, 20, 21, 22, 23, 24, 25, 26, 27 |
| 7457 | 16, 17, 18, 19, 20, 21, 22, 23, 24, 25, 26 |
| 7458 | 16, 17, 18, 19, 20, 21, 22, 23, 24, 25 |
| 7459 | 16, 17, 18, 19, 20, 21, 22, 23, 24 |
| 7460 | 16, 17, 18, 19, 20, 21, 22, 23 |
| 7461 | 16, 17, 18, 19, 20, 21, 22 |
| 7462 | 16, 17, 18, 19, 20, 21 |
| 7463 | 16, 17, 18, 19, 20 |
| 7464 | 16, 17, 18, 19 |
| 7465 | 16, 17, 18 |
| 7466 | 16, 17 |
| 7467 | 16 |

Prototype Oligonucleotides:

| Sequence | Trivial | Starting | |
|---|---|---|---|
| ID No. | Name | Position* | 5′-->3′ Sequence |
| 112 | OL(18)TRPM2 | 7456 | TGCCGCCACC CCACCCAGAG CC |
| 113 | OL(19)TRPM2 | 7461 | GCCCCTGCCG CCACCCCACC CA |

*Because this is antisense, the position numbers must be read right to left; the starting and ending
positions are defined according to the GenBank sequence with the first base of the sense strand
being base number one.
Gene: TRPM-2 (exons 1, 2, 3)
GenBank: HUMTRPM2A1/M63376
References: Pineault et al., J. Biol. Chem. 268; 5021 (1993).
HOT-SPOT 15. Range of bases included: positions 7583-7610*
Antisense Strand Sequence:
SEQ ID NO: 114: AAGCTTCCCC TCATTCGCCC ACCTGGAA

| Nucleotide Starting | Size Variants |
|---|---|
| Position* | (Number of bases in the oligomer) |
| 7583 | 16, 17, 18, 19, 20, 21, 22, 23, 24, 25, 26, 27 |
| 7584 | 16, 17, 18, 19, 20, 21, 22, 23, 24, 25, 26, 27 |
| 7585 | 16, 17, 18, 19, 20, 21, 22, 23, 24, 25, 26 |
| 7586 | 16, 17, 18, 19, 20, 21, 22, 23, 24, 25 |
| 7587 | 16, 17, 18, 19, 20, 21, 22, 23, 24 |
| 7588 | 16, 17, 18, 19, 20, 21, 22, 23 |
| 7589 | 16, 17, 18, 19, 20, 21, 22 |
| 7590 | 16, 17, 18, 19, 20, 21 |
| 7591 | 16, 17, 18, 19, 20 |
| 7592 | 16, 17, 18, 19 |
| 7593 | 16, 17, 18 |
| 7594 | 16, 17 |
| 7595 | 16 |

Prototype Oligonucleotides:

| Sequence | Trivial | Starting | |
|---|---|---|---|
| ID No. | Name | Position* | 5′-->3′ Sequence |
| 115 | OL(20)TRPM2 | 7584 | TCCCCTCATT CGCCCACCTG GA |

*Because this is antisense, the position numbers must be read right to left; the starting and ending
positions are defined according to the GenBank sequence with the first base of the sense strand
being base number one.
Gene: TRPM-2 (exons 1, 2, 3)
GenBank: HUMTRPM2A1/M63376
References: Pineault et al., J. Biol. Chem. 268; 5021 (1993).
HOT-SPOT 16. Range of bases included: positions 1190-1218*
Antisense Strand Sequence:
SEQ ID NO: 116: AGGAGAGACC CTGAGGTGCG GCGCTGGCG

| Nucleotide Starting | Size Variants |
|---|---|
| Position* | (Number of bases in the oligomer) |
| 1190 | 16, 17, 18, 19, 20, 21, 22, 23, 24, 25, 26, 27 |
| 1191 | 16, 17, 18, 19, 20, 21, 22, 23, 24, 25, 26, 27 |
| 1192 | 16, 17, 18, 19, 20, 21, 22, 23, 24, 25, 26, 27 |
| 1193 | 16, 17, 18, 19, 20, 21, 22, 23, 24, 25, 26 |
| 1194 | 16, 17, 18, 19, 20, 21, 22, 23, 24, 25 |
| 1195 | 16, 17, 18, 19, 20, 21, 22, 23, 24 |
| 1196 | 16, 17, 18, 19, 20, 21, 22, 23 |
| 1197 | 16, 17, 18, 19, 20, 21, 22 |
| 1198 | 16, 17, 18, 19, 20, 21 |
| 1199 | 16, 17, 18, 19, 20 |
| 1200 | 16, 17, 18, 19 |
| 1201 | 16, 17, 18 |
| 1202 | 16, 17 |
| 1203 | 16 |

Prototype Oligonucleotides:

| Sequence | Trivial | Starting | |
|---|---|---|---|
| ID No. | Name | Position* | 5′-->3′ Sequence |
| 117 | OL(21)TRPM2 | 1190 | ACCCTGAGGT GCGGCGCTGG CG |

*Because this is antisense, the position numbers must be read right to left; the starting and ending
positions are defined according to the GenBank sequence with the first base of the sense strand
being base number one.
Gene: TRPM-2 (exons 1, 2, 3)
GenBank: HUMTRPM2A1/M63376
References: Pineault et al., J. Biol. Chem. 268; 5021 (1993).
HOT-SPOT 17. Range of bases included: positions 1641-1668*
Antisense Strand Sequence:
SEQ ID NO: 118: GCGGTTGCGC GGCCCCTGGC TCAGCTGC

| Nucleotide Starting | Size Variants |
|---|---|
| Position* | (Number of bases in the oligomer) |
| 1641 | 16, 17, 18, 19, 20, 21, 22, 23, 24, 25, 26, 27 |
| 1642 | 16, 17, 18, 19, 20, 21, 22, 23, 24, 25, 26, 27 |
| 1643 | 16, 17, 18, 19, 20, 21, 22, 23, 24, 25, 26 |
| 6144 | 16, 17, 18, 19, 20, 21, 22, 23, 24, 25 |
| 1645 | 16, 17, 18, 19, 20, 21, 22, 23, 24 |
| 1646 | 16, 17, 18, 19, 20, 21, 22, 23 |
| 1647 | 16, 17, 18, 19, 20, 21, 22 |
| 1648 | 16, 17, 18, 19, 20, 21 |
| 1649 | 16, 17, 18, 19, 20 |
| 1650 | 16, 17, 18, 19 |
| 1651 | 16, 17, 18 |
| 1652 | 16, 17 |
| 1653 | 16 |

Prototype Oligonucleotides:

| Sequence | Trivial | Starting | |
|---|---|---|---|
| ID No. | Name | Position* | 5′-->3′ Sequence |
| 119 | OL(22)TRPM2 | 1643 | TTGCGCGGCC CCTGGCTCAG CT |

*Because this is antisense, the position numbers must be read right to left; the starting and ending
positions are defined according to the GenBank sequence with the first base of the sense strand
being base number one.
Gene: TRPM-2 (exons 1, 2, 3)
GenBank: HUMTRPM2A1/M63376
References: Pineault et al., J. Biol. Chem. 268; 5021 (1993).
HOT-SPOT 18. Range of bases included: positions 45324578*
Antisense Strand Sequence:
SEQ ID NO: 120: GAAACCCCTC CCCCTGCACT CCACCCCCAC CATGCCCCTC
CCAAGAA

| Nucleotide Starting | Size Variants |
|---|---|
| Position* | (Number of bases in the oligomer) |
| 4532 | 16, 17, 18, 19, 20, 21, 22, 23, 24, 25, 26, 27 |
| 4533 | 16, 17, 18, 19, 20, 21, 22, 23, 24, 25, 26, 27 |
| 4534 | 16, 17, 18, 19, 20, 21, 22, 23, 24, 25, 26, 27 |
| 4535 | 16, 17, 18, 19, 20, 21, 22, 23, 24, 25, 26, 27 |
| 4536 | 16, 17, 18, 19, 20, 21, 22, 23, 24, 25, 26, 27 |
| 4537 | 16, 17, 18, 19, 20, 21, 22, 23, 24, 25, 26, 27 |
| 4538 | 16, 17, 18, 19, 20, 21, 22, 23, 24, 25, 26, 27 |
| 4539 | 16, 17, 18, 19, 20, 21, 22, 23, 24, 25, 26, 27 |
| 4540 | 16, 17, 18, 19, 20, 21, 22, 23, 24, 25, 26, 27 |
| 4541 | 16, 17, 18, 19, 20, 21, 22, 23, 24, 25, 26, 27 |
| 4542 | 16, 17, 18, 19, 20, 21, 22, 23, 24, 25, 26, 27 |
| 4543 | 16, 17, 18, 19, 20, 21, 22, 23, 24, 25, 26, 27 |
| 4544 | 16, 17, 18, 19, 20, 21, 22, 23, 24, 25, 26, 27 |
| 4545 | 16, 17, 18, 19, 20, 21, 22, 23, 24, 25, 26, 27 |
| 4546 | 16, 17, 18, 19, 20, 21, 22, 23, 24, 25, 26, 27 |
| 4547 | 16, 17, 18, 19, 20, 21, 22, 23, 24, 25, 26, 27 |
| 4548 | 16, 17, 18, 19, 20, 21, 22, 23, 24, 25, 26, 27 |
| 4549 | 16, 17, 18, 19, 20, 21, 22, 23, 24, 25, 26, 27 |
| 4550 | 16, 17, 18, 19, 20, 21, 22, 23, 24, 25, 26, 27 |
| 4551 | 16, 17, 18, 19, 20, 21, 22, 23, 24, 25, 26, 27 |
| 4552 | 16, 17, 18, 19, 20, 21, 22, 23, 24, 25, 26, 27 |

Prototype Oligonucleotides:

| Sequence | Trivial | Starting | |
|---|---|---|---|
| ID No. | Name | Position* | 5′-->3′ Sequence |
| 121 | OL(23)TRPM2 | 4537 | CCACCCCCAC CATGCCCCTC CC |
| 122 | OL(24)TRPM2 | 4548 | CCCCCTGCAC TCCACCCCCA CC |

*Because this is antisense, the position numbers must be read right to left; the starting and ending
positions are defined according to the GenBank sequence with the first base of the sense strand
being base number one.
The Human TRPM-2 (exon 4) Gene
Gene: TRPM-2 (exon 4)
GenBank: HUMTRPM2A2/M63377
References: Pineault et al., J. Biol. Chem. 268; 5021 (1993).
HOT-SPOT 1. Range of bases included: positions 315-340*
Antisense Strand Sequence:
SEQ ID NO: 123: GTGTCCCCTT TTCACCTGGC GGCCAA

| Nucleotide Starting | Size Variants |
|---|---|
| Position* | (Number of bases in the oligomer) |
| 315 | 16, 17, 18, 19, 20, 21, 22, 23, 24, 25, 26, 27 |
| 316 | 16, 17, 18, 19, 20, 21, 22, 23, 24, 25, 26, 27 |
| 317 | 16, 17, 18, 19, 20, 21, 22, 23, 24, 25, 26, 27 |
| 318 | 16, 17, 18, 19, 20, 21, 22, 23, 24, 25, 26, 27 |
| 319 | 16, 17, 18, 19, 20, 21, 22, 23, 24, 25, 26, 27 |
| 320 | 16, 17, 18, 19, 20, 21, 22, 23, 24, 25, 26, 27 |

Prototype Oligonucleotides:

| Sequence | Trivial | Starting | |
|---|---|---|---|
| ID No. | Name | Position* | 5′-->3′ Sequence |
| 124 | OL(25)TRPM2 | 315 | TCCCCT′TTTC ACCTGGCGGC CAA |

*Because this is antisense, the position numbers must be read right to left; the starting and ending
positions are defined according to the GenBank sequence with the first base of the sense strand
being base number one.
The Human TRPM-2 (exon 5, 6) Gene
Gene: TRPM-2 (exon 5, 6)
GenBank: HUMTRPM2A3/M63378
References: Pineault et al., J. Biol. Chem. 268; 5021 (1993).
HOT-SPOT 1. Range of bases included: positions 148-173 *
Antisense Strand Sequence:
SEQ ID NO: 125: GGACCAGCCC CCAGCACCCC ATCTGT

| Nucleotide Starting | Size Variants |
|---|---|
| Position* | (Number of bases in the oligomer) |
| 148 | 16, 17, 18, 19, 20, 21, 22, 23, 24, 25, 26 |
| 149 | 16, 17, 18, 19, 20, 21, 22, 23, 24, 25 |
| 150 | 16, 17, 18, 19, 20, 21, 22, 23, 24 |
| 151 | 16, 17, 18, 19, 20, 21, 22, 23 |
| 152 | 16, 17, 18, 19, 20, 21, 22 |
| 153 | 16, 17, 18, 19, 20, 21 |
| 154 | 16, 17, 18, 19, 20 |
| 155 | 16, 17, 18, 19 |
| 156 | 16, 17, 18 |
| 157 | 16, 17 |
| 158 | 16 |

Prototype Oligonucleotides:

| Sequence | Trivial | Starting | |
|---|---|---|---|
| ID No. | Name | Position* | 5′-->3′ Sequence |
| 126 | OL(26)TRPM2 | 152 | GGACCAGCCC CCAGCACCCC AT |

*Because this is antisense, the position numbers must be read right to left; the starting and ending
positions are defined according to the GenBank sequence with the first base of the sense strand
being base number one.
Gene: TRPM-2 (exon 5, 6)
GenBank: HUMTRPM2A3/M63378
References: Pineault et al., J. Biol. Chem. 268; 5021 (1993).
HOT-SPOT 2. Range of bases included: positions 548-580*
Antisense Strand Sequence:
SEQ ID NO: 127: CATCAAGCTG CGGACGATGC GGGACTTGGG AAA

| Nucleotide Starting | Size Variants |
|---|---|
| Position* | (Number of bases in the oligomer) |
| 548 | 16, 17, 18, 19, 20, 21, 22, 23, 24, 25, 26, 27 |
| 549 | 16, 17, 18, 19, 20, 21, 22, 23, 24, 25, 26, 27 |
| 550 | 16, 17, 18, 19, 20, 21, 22, 23, 24, 25, 26, 27 |
| 551 | 16, 17, 18, 19, 20, 21, 22, 23, 24, 25, 26, 27 |
| 552 | 16, 17, 18, 19, 20, 21, 22, 23, 24, 25, 26, 27 |
| 553 | 16, 17, 18, 19, 20, 21, 22, 23, 24, 25, 26, 27 |
| 554 | 16, 17, 18, 19, 20, 21, 22, 23, 24, 25, 26, 27 |
| 555 | 16, 17, 18, 19, 20, 21, 22, 23, 24, 25, 26 |
| 556 | 16, 17, 18, 19, 20, 21, 22, 23, 24, 25 |
| 557 | 16, 17, 18, 19, 20, 21, 22, 23, 24 |
| 558 | 16, 17, 18, 19, 20, 21, 22, 23 |
| 559 | 16, 17, 18, 19, 20, 21, 22 |
| 560 | 16, 17, 18, 19, 20, 21 |
| 561 | 16, 17, 18, 19, 20 |
| 562 | 16, 17, 18, 19 |
| 563 | 16, 17, 18 |
| 564 | 16, 17 |
| 565 | 16 |

Prototype Oligonucleotides:

| Sequence | Trivial | Starting | |
|---|---|---|---|
| ID No. | Name | Position* | 5′-->3′ Sequence |
| 128 | OL(27)TRPM2 | 551 | TGCGGACGAT GCGGGACTTG GG |

*Because this is antisense, the position numbers must be read right to left; the starting and ending
positions are defined according to the GenBank sequence with the first base of the sense strand
being base number one.
Gene: TRPM-2 (exon 5, 6)
GenBank: HUMTRPM2A3/M63378
References: Pineault et al., J. Biol. Chem. 268; 5021 (1993).
HOT-SPOT 3. Range of bases included: positions 686-718*
Antisense Strand Sequence:
SEQ ID NO: 129: TCGTATGAAT TCTGTTGGCG GGTGCTGGAA GGC

| Nucleotide Starting | Size Variants |
|---|---|
| Position* | (Number of bases in the oligomer) |
| 686 | 16, 17, 18, 19, 20, 21, 22, 23, 24, 25, 26, 27 |
| 687 | 16, 17, 18, 19, 20, 21, 22, 23, 24, 25, 26, 27 |
| 688 | 16, 17, 18, 19, 20, 21, 22, 23, 24, 25, 26, 27 |
| 689 | 16, 17, 18, 19, 20, 21, 22, 23, 24, 25, 26, 27 |
| 690 | 16, 17, 18, 19, 20, 21, 22, 23, 24, 25, 26, 27 |
| 691 | 16, 17, 18, 19, 20, 21, 22, 23, 24, 25, 26, 27 |
| 692 | 16, 17, 18, 19, 20, 21, 22, 23, 24, 25, 26, 27 |

Prototype Oligonucleotides:

| Sequence | Trivial | Starting | |
|---|---|---|---|
| ID No. | Name | Position* | 5′-->3′ Sequence |
| 130 | OL(28)TRPM2 | 686 | CTGTTGGCGG GTGCTGGAAG GC |

*Because this is antisense, the position numbers must be read right to left; the starting and ending
positions are defined according to the GenBank sequence with the first base of the sense strand
being base number one.
Gene: TRPM-2 (exon 5, 6)
GenBank: HUMTRPM2A3/M63378
References: Pineault et al., J. Biol. Chem. 268; 5021 (1993).
HOT-SPOT 4. Range of bases included: positions 239-263*
Antisense Strand Sequence:
SEQ ID NO: 131: CCACGGCTCC TGGCTCCCAC CCCCT

| Nucleotide Starting | Size Variants |
|---|---|
| Position* | (Number of bases in the oligomer) |
| 239 | 16, 17, 18, 19, 20, 21, 22, 23, 24, 25 |
| 240 | 16, 17, 18, 19, 20, 21, 22, 23, 24 |
| 241 | 16, 17, 18, 19, 20, 21, 22, 23 |
| 242 | 16, 17, 18, 19, 20, 21, 22 |
| 243 | 16, 17, 18, 19, 20, 21 |
| 244 | 16, 17, 18, 19, 20 |
| 245 | 16, 17, 18, 19 |
| 246 | 16, 17, 18 |
| 247 | 16, 17 |
| 248 | 16 |

Prototype Oligonucleotides:

| Sequence | Trivial | Starting | |
|---|---|---|---|
| ID No. | Name | Position* | 5′-->3′ Sequence |
| 132 | OL(29)TRPM2 | 240 | CACGGCTCCT GGCTCCCACC CCC |

*Because this is antisense, the position numbers must be read right to left; the starting and ending
positions are defined according to the GenBank sequence with the first base of the sense strand
being base number one.
Gene: TRPM-2 (exon 5, 6)
GenBank: HUMTRPM2A3/M63378
References: Pineault et al., J. Biol. Chem. 268; 5021 (1993).
HOT-SPOT 5. Range of bases included: positions 416-454*
Antisense Strand Sequence:
SEQ ID NO: 133: GTCTATGATG CTGGACGCGC GGCTGAAGTG GTCCTGCAT

| Nucleotide Starting | Size Variants |
|---|---|
| Position* | (Number of bases in the oligomer) |
| 416 | 16, 17, 18, 19, 20, 21, 22, 23, 24, 25, 26, 27 |
| 417 | 16, 17, 18, 19, 20, 21, 22, 23, 24, 25, 26, 27 |
| 418 | 16, 17, 18, 19, 20, 21, 22, 23, 24, 25, 26, 27 |
| 419 | 16, 17, 18, 19, 20, 21, 22, 23, 24, 25, 26, 27 |
| 420 | 16, 17, 18, 19, 20, 21, 22, 23, 24, 25, 26, 27 |
| 421 | 16, 17, 18, 19, 20, 21, 22, 23, 24, 25, 26, 27 |
| 422 | 16, 17, 18, 19, 20, 21, 22, 23, 24, 25, 26, 27 |
| 423 | 16, 17, 18, 19, 20, 21, 22, 23, 24, 25, 26, 27 |
| 424 | 16, 17, 18, 19, 20, 21, 22, 23, 24, 25, 26, 27 |
| 425 | 16, 17, 18, 19, 20, 21, 22, 23, 24, 25, 26, 27 |
| 426 | 16, 17, 18, 19, 20, 21, 22, 23, 24, 25, 26, 27 |
| 427 | 16, 17, 18, 19, 20, 21, 22, 23, 24, 25, 26, 27 |
| 428 | 16, 17, 18, 19, 20, 21, 22, 23, 24, 25, 26, 27 |
| 429 | 16, 17, 18, 19, 20, 21, 22, 23, 24, 25, 26 |
| 430 | 16, 17, 18, 19, 20, 21, 22, 23, 24, 25 |
| 431 | 16, 17, 18, 19, 20, 21, 22, 23, 24 |
| 432 | 16, 17, 18, 19, 20, 21, 22, 23 |
| 433 | 16, 17, 18, 19, 20, 21, 22 |
| 434 | 16, 17, 18, 19, 20, 21 |
| 435 | 16, 17, 18, 19, 20 |
| 436 | 16, 17, 18, 19 |
| 437 | 16, 17, 18 |
| 438 | 16, 17 |

Prototype Oligonucleotides:

| Sequence | Trivial | Starting | |
|---|---|---|---|
| ID No. | Name | Position* | 5′-->3′ Sequence |
| 134 | OL(30)TRPM2 | 424 | GCTGGACGCG CGGCTGAAGT GG |

*Because this is antisense, the position numbers must be read right to left; the starting and ending
positions are defined according to the GenBank sequence with the first base of the sense strand
being base number one.

TABLE 5
Prototype Primary Start Site Steric Hindrance Antisense Oligos and Prototype Secondary Start Site
Steric Hindrance Antisense Oligos: Single and Duel Antisense Oligo Combinations Targeting CLU34
for Clinical Indications Involving Therapeutic Inhibition of Clusterin Induced Resistance to
Apoptosis

| | Primary Translational | SEQ | Secondary Translational | Seq |
|---|---|---|---|---|
| Content of Row | Start Site | IDs | Start Sites | IDs |
| Hotspot for Primary Translational | GCAAACCTGC ATGACTCACG | 4 | Not Applicable | — |
| Start Site Region | CCCAAAGAAT GC | | | |
| Prototype Primary Translational | A(1)CLU34: TGCATGACTC | 5 | Not Applicable | — |
| Start Site Antisense Oligo #1 | ACGCCCAAAG AA | | | |
| Prototype Primary Translational | A(2)CLU34: GCAAACCTGC | 6 | Not Applicable | — |
| Start Site Antisense Oligo #2 | ATGACTCACG CC | | | |
| Hotspot for Secondary Translational | Not Applicable | — | CAGCAGAGTC TTCATCATGC | 21 |
| Start Site Region | | | CTCCAATTCT TG | |
| Combination #1 Prototype Primary | A(1)CLU34: TGCATGACTC | 5 | A(1)CLU35: TTCATCATGC | |
| and Secondary Translational Start | ACGCCCAAAG AA | | CTCCAATTCT GG | 22 |
| Site Antisense Oligos | | | | |
| Combination #2 Prototype Primary | A(2)CLU34: GCAAACCTGC | 6 | A(1)CLU35: TTCATCATGC | |
| and Secondary Translational Start | ATGACTCACG CC | | CTCCAATTCT GG | 22 |
| Site Antisense Oligos | | | | |
| Combination #3 Prototype Primary | A(1)CLU34: TGCATGACTC | 5 | A(2)CLU35: CAGCAGCAGA | |
| and Secondary Translational Start | ACGCCCAAAG AA | | GTCTTCATCA TG | 23 |
| Site Antisense Oligos | | | | |
| Combination #4 Prototype Primary | A(2)CLU34: GCAAACCTGC | 6 | A(2)CLU35: CAGCAGCAGA | |
| and Secondary Translational Start | ATGACTCACG CC | | GTCTTCATCA TG | 23 |
| Site Antisense Oligos | | | | |

TABLE 6
Prototype Primary Start Site Steric Hindrance Antisense Oligos and Prototype Secondary Start
Site Steric Hindrance Antisense Oligos: Single and Duel Antisense Oligo Combinations
Targeting CLU35 for Clinical Indications Involving Therapeutic Inhibition of Clusterin
Induced Resistance to Apoptosis

| | Primary Translational | SEQ ID | Secondary Translational | SEQ ID |
|---|---|---|---|---|
| Content of Row | Start Site | NOS: | Start Sites | NOS: |
| Hotspot for Primary Transitional | CAGCAGAGTC TTCATCATGC | 21 | Not Applicable | — |
| Start Site Region | CTCCAATTCT TG | | | |
| Prototype Primary Transitional Start | A(1)CLU35: TTCATCATGC | 22 | Not Applicable | — |
| Site Antisense Oligo #1 | CTCCAATTCT GG | | | |
| Prototype Primary Transitional Start | A(2)CLU35: CAGCAGCAGA | 23 | Not Applicable | — |
| Site Antisense Oligo #2 | GTCTTCATCA TG | | | |
| Hotspot for Secondary Transitional | Not Applicable | — | CCCTGATTGG ACATTTCCTG | 7 |
| Start Site Region | | | GAGCTCATTGT CTGA | |
| Combination #1 Prototype Primary and | A(1)CLU35: TTCATCATGC | 22 | A(3)CLU34: GGACATTTCC | 8 |
| Secondary Transitional Start Site | CTCCAATTCT GG | | TGGAGCTCAT TG | |
| Antisense Oligos | | | | |
| Combination #2 Prototype Primary and | A(1)CLU35: TTCATCATGC | 22 | A(4)CLU34: CCCTGATTGG | 9 |
| Secondary Transitional Start Site | CTCCAATTCT GG | | ACATTTCCTG GA | |
| Antisense Oligos | | | | |
| Combination #3 Prototype Primary and | A(2)CLU35: CAGCAGCAGA | 23 | A(3)CLU34: GGACATTTCC | 8 |
| Secondary Transitional Start Site | GTCTTCATCA TG | | TGGAGCTCAT TG | |
| Antisense Oligos | | | | |
| Combination #4 Prototype Primary and | A(2)CLU35: CAGCAGCAGA | 23 | A(4)CLU34: CCCTGATTGG | 9 |
| Secondary Transitional Start Site | GTCTTCATCA TG | | ACATTTCCTG GA | |
| Antisense Oligos | | | | |
| Hotspot for Secondary Transitional | Not Applicable | — | CTCAGAGGGC CATCATGGTC | 10 |
| Start Site Region | | | TCATTGCACA CTCCTGGGAG | |
| Combination #5 Prototype Primary and | A(1)CLU35: TTCATCATGC | 22 | A(5)CLU34: GGGCCATCAT | 11 |
| Alternative Secondary Transitional | CTCCAATTCT GG | | GGTCTCATTG CA | |
| Start Site Antisense Oligos | | | | |
| Combination #5 Prototype Primary and | A(1)CLU35: TTCATCATGC | 22 | A(6)CLU34: CAGAGGGCCA | 12 |
| Alternative Secondary Transitional | CTCCAATTCT GG | | TCATGGTCT | |
| Start Site Antisense Oligos | | | | |
| Combination #5 Prototype Primary and | A(2)CLU35: CAGCAGCAGA | 23 | A(5)CLU34: GGGCCATCAT | 11 |
| Alternative Secondary Transitional | GTCTTCATCA TG | | GGTCTCATTG CA | |
| Start Site Antisense Oligos | | | | |

TABLE 7
Prototype Primary Start Site Steric Hindrance Antisense Oligos and Prototype Secondary Start
Site Steric Hindrance Antisense Oligos: Single and Duel Antisense Oligo Combinations
Targeting CLU36 for Clinical Indications Involving Therapeutic Inhibition of Clusterin Induced
Apoptosis

| | Primary Translational | SEQ ID | Secondary Translational | SEQ ID |
|---|---|---|---|---|
| Content of Row | Start Site | NO: | Start Sites | NO: |
| Hotspot for Primary Transitional | CAGCAGUCCC ATCTGGACAC | 33 | Not Applicable | — |
| Start Site Region | TCAGTAGCAC ATTAACAAGG | | | |
| | AGTGCCTCTC A | | | |
| Prototype Primary Transitional | A(1)CLU36: CACATTAACA | 34 | Not Applicable | — |
| Start Site Antisense Oligo #1 | AGGAGTGCCT CTC | | | |
| Prototype Primary Transitional | A(2)CLU36: TCTGGACACT | 35 | Not Applicable | — |
| Start Site Antisense Oligo #2 | CAGTAGCACA TTA | | | |
| Hotspot for Secondary Transitional | Not Applicable | — | CAGCAGAGTC TTCATCATGC | 21 |
| Start Site Region | | | CTCCAATTCT TG | |
| Combination #1 Prototype Primary | A(1)CLU36: CACATTAACA | 34 | A(1)CLU35: TTCATCATGC | 22 |
| and Secondary Transitional Start | AGGAGTGCCT CTC | | CTCCAATTCT GG | |
| Site Antisense Oligos | | | | |
| Combination #2 Prototype Primary | A(1)CLU36: CACATTAACA | 34 | A(2)CLU35: CAGCAGCAGA | 23 |
| and Secondary Transitional Start | AGGAGTGCCT CTC | | GTCTTCATCA TG | |
| Site Antisense Oligos | | | | |
| Combination #3 Prototype Primary | A(2)CLU36: TCTGGACACT | 35 | A(1)CLU35: TTCATCATGC | 22 |
| and Secondary Transitional Start | CAGTAGCACA TTA | | CTCCAATTCT GG | |
| Site Antisense Oligos | | | | |
| Combination #4 Prototype Primary | A(2)CLU36: TCTGGACACT | 35 | A(2)CLU35: CAGCAGCAGA | 23 |
| and Secondary Transitional Start | CAGTAGCACA TTA | | GTCTTCATCA TG | |
| Site Antisense Oligos | | | | |

TABLE 8
Prototype Alternative Primary Start Site Steric Hindrance Antisense Oligos and Prototype
Secondary Start Site Steric Hindrance Antisense Oligos: Single and Duel Antisense Oligo
Combinations Targeting CLU36 for Clinical Indications Involving Therapeutic Inhibition
of Clusterin Induced Apoptosis

| | Primary Translational | SEQ ID | Secondary Translational | SEQ ID |
|---|---|---|---|---|
| Content of Row | Start Site | NO: | Start Sites | NO: |
| Hotspot for Alternative Primary | GTCTTTGCAC GCCTCCATTT | 36 | Not Applicable | — |
| Transitional Start Site Region | GCCATCACAG AACCAACAGG | | | |
| | ACGATGGG | | | |
| Prototype Alternative Primary | A(3)CLU36: CGCCTCCATT | 37 | Not Applicable | — |
| Transitional Start Site Antisense | TGCCATCACA GA | | | |
| Oligo #1 | | | | |
| Prototype Alternative Primary | A(4)CLU36: CACGCCTCCA | 38 | Not Applicable | — |
| Transitional Start Site Antisense | TTTGCCATCA | | | |
| Oligo #2 | | | | |
| Hotspot for Secondary Transitional | Not Applicable | — | CAGCAGAGTC TTCATCATGC | 21 |
| Start Site Region | | | CTCCAATTCT TG | |
| Combination #1 Prototype | A(3)CLU36: CGCCTCCATT | 37 | A(1)CLU35: TTCATCATGC | 22 |
| Alternative Primary and Secondary | TGCCATCACA GA | | CTCCAATTCT GG | |
| Transitional Start Site Antisense | | | | |
| Oligos | | | | |
| Combination #2 Prototype | A(3)CLU36: CGCCTCCATT | 37 | A(2)CLU35: CAGCAGCAGA | 23 |
| Alternative Primary and Secondary | TGCCATCACA GA | | GTCTTCATCA TG | |
| Transitional Start Site Antisense | | | | |
| Oligos | | | | |
| Combination #3 Prototype | A(4)CLU36: CACGCCTCCA | 38 | A(1)CLU35: TTCATCATGC | 22 |
| Alternative Primary and Secondary | TTTGCCATCA | | CTCCAATTCT GG | |
| Transitional Start Site Antisense | | | | |
| Oligos | | | | |
| Combination #4 Prototype | A(4)CLU36: CACGCCTCCA | 38 | A(2)CLU35: CAGCAGCAGA | 23 |
| Alternative Primary and Secondary | TTTGCCATCA | | GTCTTCATCA TG | |
| Transitional Start Site Antisense | | | | |
| Oligos | | | | |

TABLE 9
Prototype Primary Start Site Steric Hindrance Antisense Oligos and Prototype
Secondary Start Site Steric Hindrance Antisense Oligos: Single and Duel Antisense
Oligo Combinations Targeting Alternatively Spliced CLU34 (Primary Translational
Start Site in Exon 3) for Clinical Indications Involving Therapeutic Inhibition
of Clusterin Induced Apoptosis

| | Primary Translational | SEQ ID | Secondary Translational | SEQ ID |
|---|---|---|---|---|
| Content of Row | Start Site | NO: | Start Sites | NO: |
| Hotspot for Primary Transitional | CCCTGATTGG ACATTTCCTG | 7 | Not Applicable | — |
| Start Site Region | GAGCTCATTGT CTGA | | | |
| Prototype Primary Transitional | A(3)CLU34: GGACATTTCC | 8 | Not Applicable | — |
| Start Site Antisense Oligo #1 | TGGAGCTCAT TG | | | |
| Prototype Primary Transitional | A(4)CLU34: CCCTGATTGG | 9 | Not Applicable | — |
| Start Site Antisense Oligo #2 | ACATTTCCTG GA | | | |
| Hotspot for Secondary Transitional | Not Applicable | — | CTCAGAGGGC CATCATGGTC | 10 |
| Start Site Region | | | TCATTGCACA CTCCTGGGAG | |
| Combination #1 Prototype Primary | A(3)CLU34: GGACATTTCC | 8 | A(5)CLU34: GGGCCATCAT | 11 |
| and Secondary Transitional Start | TGGAGCTCAT TG | | GGTCTCATTG CA | |
| Site Antisense Oligos | | | | |
| Combination #2 Prototype Primary | A(3)CLU34: GGACATTTCC | 8 | A(6)CLU34: CAGAGGGCCA | 12 |
| and Secondary Transitional Start | TGGAGCTCAT TG | | TCATGGTCT | |
| Site Antisense Oligos | | | | |
| Combination #3 Prototype Primary | A(4)CLU34: CCCTGATTGG | 9 | A(5)CLU34: GGGCCATCAT | 11 |
| and Secondary Transitional Start | ACATTTCCTG GA | | GGTCTCATTG CA | |
| Site Antisense Oligos | | | | |
| Combination #4 Prototype Primary | A(4)CLU34: CCCTGATTGG | 9 | A(6)CLU34: CAGAGGGCCA | 12 |
| and Secondary Transitional Start | ACATTTCCTG GA | | TCATGGTCT | |
| Site Antisense Oligos | | | | |

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A composition comprising at least one agent which inhibits p53 expression, wherein said agent comprises a sequence which hybridizes to the p53 encoding nucleic acid in a biologically acceptable carrier wherein said oligonucleotide has a sequence corresponding to at least one sequence provided depicted in FIG. 1, wherein said sequence excludes those numbered 17, 18 and 34-39.

2. A composition comprising at least two agents which inhibit p53 expression, wherein said agent comprises a sequence which hybridizes to the p53 encoding nucleic acid in a biologically acceptable carrier wherein said oligonucleotide has a sequence corresponding to a sequence selected from the group consisting of sequences shown in FIG. 1 wherein said sequence excludes those numbered 17, 18 and 34-39, and SEQ ID NOS: 135-141.

3. The composition of claim 1 further comprising at least one of an antioxidant, an anti-inflammatory, a redox modifier, an interferon and a cytokine.

4. The composition of claim 1, wherein said nucleic acid is present in an expression vector.

5. The composition of claim 4, further comprising a carrier which facilitates cellular uptake.

6. The composition of claim 1, comprising a CPP selected from the group consisting of RX₈B and R₆Pen where R is arginine, X is 6-aminohexanoic acid, B is beta-alanine and Pen is the peptide Penetratin, RQLKIWFQNRRMKWKK.

7. The composition of claim 1 further comprising one or more oligonucleotides provided in Table 2.

8. A method for inhibiting p53 expression in a cell or tissue comprising: contacting said cells or tissue with an effective amount of at least one agent as claimed in claim 7 which inhibits p53 expression under conditions whereby said agent enters said cells and reduces p53 expression relative to untreated cell, said oligo comprising a CPP selected from the group consisting of RX₈B and R₆Pen where R is arginine, X is 6-aminohexanoic acid, B is beta-alanine and Pen is the peptide Penetratin, RQLKIWFQNRRMKWKK.

9. The method of claim 8, wherein said agent modulates apoptosis in said cell.

10. The method of claim 8, for the treatment of a disorder listed in Table 1.

11. The method of claim 8, wherein said inhibition of p53 expression produces a therapeutic benefit in said normal cells, said benefit comprising increasing the viability of constitutive self renewing normal tissue composes of a cell type selected from the group consisting of gastrointestinal epithelial cells, skin cells and bone marrow cells.

12. The method as claimed in claim 9, for the treatment of disease wherein said disease is selected from the group consisting of Cancer, AIDS, Alzheimer's disease, Amyotrophic lateral sclerosis, Atherosclerosis, Autoimmune Diseases, Cerebellar degeneration, Cancer, Diabetes Mellitus, Glomerulonephritis, Heart Failure, Macular Degeneration, Multiple sclerosis, Myelodysplastic syndromes, Parkinson's disease, Prostatic hyperplasia, Psoriasis, Asthma, Retinal Degeneration, Retinitis pigmentosa, Rheumatoid arthritis, Rupture of atherosclerotic plaques, Systemic lupus erythematosis, Ulcerative colitis, viral infection, ischemia reperfusion injury, cardiohypertrophy, and Diamond Black Fan anemia.

13. The method as claimed in claim 12, for the treatment of Diamond blackfan anemia.

14. An oligonucleotide effective to down modulate clusterin expression in a target cell comprising at least one oligonucleotide provided in Tables 4A, 4B and 4C, said oligonucleotide optionally being present in a biologically acceptable carrier, said oligonucleotide optionally comprising a CPP selected from the group consisting of RX₈B and R₆Pen where R is arginine, X is 6-aminohexanoic acid, B is beta-alanine and Pen is the peptide Penetratin, RQLKIWFQNRRMKWKK.

15. A pair of oligonucleotides effective to down modulate clusterin expression in a target cells, said pair of oligonucleotides being selected from the groups of pairs provided in Tables 5, 6, 7, 8 and 9, said oligonucleotides optionally being present in a biologically acceptable carrier.

16. The oligonucleotides of claim 15 comprising a morpholino backbone modification and a CPP.

17. A method for modulating aberrant apoptosis in a target cell comprising administration of at least one oligonucleotide as claimed in claim 14, said oligonucleotide being effective to down modulate clusterin expression thereby modulating apoptosis in said target cell.

18. The method of claim 17, wherein said target cell is a cancer cell and apoptosis in increased.

19. The method of claim 18, wherein said cancer is selected from the group consisting of brain cancer, lung cancer, ovarian cancer, breast cancer, testicular cancer, kidney cancer, liver cancer, skin cancer, pancreatic cancer, esophageal cancer, stomach cancer, bladder cancer, uterine cancer, prostate cancer, glaucomas, sarcomas, myelomas, lymphomas, and leukemias.

20. The method of claim 19 for the treatment of prostate cancer comprising administration of a pair of oligos directed to clusterin, said pair being selected from the group consisting of the pairs of oligos provided in Tables, 5, 6, 7, 8, or 9.

21. The method of claim of claim 17, comprising administration of a pair of oligonucleotides which down modulate clusterin expression, one member of said pair being selected from the group of oligonucleotides in table 4D and the other member being selected from the oligos presented in Tables 5, 6, 7, 8, or 9.

22. A method for inhibiting p53 expression in a patient having Del (5q) MDS with thalidomide or lenalidomide resistance comprising: contacting said cells or tissue in said patient with an effective amount of at least one agent as claimed in claim 1 which inhibits p53 expression under conditions whereby said agent enters said cells and reduces p53 expression in an amount effective reduce cytopenia or thrombocytopenia in said patient.
